SEQUENCE LISTING (455 sequences)

SEQ ID NO: 1 - DNA, 127 bp, Artificial Sequence
  Illumina Miseq
  misc_feature (25)..(32): n is a, c, g, or t
ccactgggtctggtcaatcacgccnnnnnnnnaaccattagctgacattccgctctaggatccggagtcaccatatccataagatatgaacgcattgcccggcccgctcgattccatgaactttccc

SEQ ID NO: 2 - DNA, 55 bp, Artificial Sequence
  DNA ligation Adapter
  misc_feature (11)..(22): n is a, c, g, or t
  misc_feature (55)..(55): c is dideoxycytidine
ggactccaatnnnnnnnnnnnnacgctaagaaagatcggaagagcacacgtctgc

SEQ ID NO: 3 - DNA, 11 bp, Artificial Sequence
  DNA ligation Adapter
attggagtcct

SEQ ID NO: 4 - DNA, 62 bp, Artificial Sequence
  Reverse Transcription Oligos
  misc_feature (30)..(39): n is a, c, g, or t
  misc_feature (61)..(61): n is a, c, g, or t
  misc_feature (62)..(62): v is a, g, or c
cgactcactatagggctggaattctgacgnnnnnnnnnnacgttttttttttttttttttnv

SEQ ID NO: 5 - DNA, 56 bp, Artificial Sequence
  Reverse Transcription Oligos
  misc_feature (1)..(1): c is 5methyl-deoxyisocytidine
  misc_feature (2)..(3): g is deoxyiso guanosine
  misc_feature (38)..(47): n is a, c, g, or t
  misc_feature (54)..(56): g is riboguanosine
cggtaatacgactcactatagggctggaattctgacgnnnnnnnnnnatctgcggg

SEQ ID NO: 6 - DNA, 98 bp, Artificial Sequence
  Target Enrichment Oligos
agcagtggtatcaacgcagagtcaagcagaagacggcatacgagattccgaaacgtgactggagttcagacgtgtgctcttccgatctttcttagcgt

SEQ ID NO: 7 - DNA, 115 bp, Artificial Sequence
  Target Enrichment Oligos
gtgagtgatggttgaggatgtgtgcaagcagaagacggcatacgagattacgtacggtgactggagttcagacgtgtgctcttccgatctcgactcactatagggctggaattct

SEQ ID NO: 8 - DNA, 84 bp, Artificial Sequence
  uPCR Primers
  misc_feature (59)..(63): n is a, c, g, or t
aatgatacggcgaccaccgagatctacactctttccctacacgacgctcttccgatctnnnnnaatgtacagtattgcgttttg

SEQ ID NO: 9 - DNA, 23 bp, Artificial Sequence
  uPCR Primers
aagcagtggtatcaacgcagagt

SEQ ID NO: 10 - DNA, 24 bp, Artificial Sequence
  uPCR Primers
gtgagtgatggttgaggatgtgtg

SEQ ID NO: 11 - DNA, 47 bp, Artificial Sequence
  SPE Primer Pool
aatgtacagtattgcgttttgagccccaagtcctatgagaacctctg

SEQ ID NO: 12 - DNA, 46 bp, Artificial Sequence
  SPE Primer Pool
aatgtacagtattgcgttttgtggcaccagcgatcaggtcctttat

SEQ ID NO: 13 - DNA, 47 bp, Artificial Sequence
  SPE Primer Pool
aatgtacagtattgcgttttgctgagtggagtcacagcggagatagt

SEQ ID NO: 14 - DNA, 51 bp, Artificial Sequence
  SPE Primer Pool
aatgtacagtattgcgttttgtgttccaccagtaacaacagttgaatgtcc

SEQ ID NO: 15 - DNA, 51 bp, Artificial Sequence
  SPE Primer Pool
aatgtacagtattgcgttttggtgtgaggaacatactagtgctttgcaagt

SEQ ID NO: 16 - DNA, 50 bp, Artificial Sequence
  SPE Primer Pool
aatgtacagtattgcgttttgttcaaagttgggtctgcttcagtccaaag

SEQ ID NO: 17 - DNA, 48 bp, Artificial Sequence
  SPE Primer Pool
aatgtacagtattgcgttttgcccccagcttcttctctctgcactaag

SEQ ID NO: 18 - DNA, 51 bp, Artificial Sequence
  SPE Primer Pool
aatgtacagtattgcgttttggccttcccaacatgcattctaacttcttcc

SEQ ID NO: 19 - DNA, 51 bp, Artificial Sequence
  SPE Primer Pool
aatgtacagtattgcgttttgccagctactctcaaaatcagcatcctttgg

SEQ ID NO: 20 - DNA, 51 bp, Artificial Sequence
  SPE Primer Pool
aatgtacagtattgcgttttgccagtccttctgtgagtctatcctcagttc

SEQ ID NO: 21 - DNA, 49 bp, Artificial Sequence
  SPE Primer Pool
aatgtacagtattgcgttttgagagcgaaccaagaatgcctgtttacag

SEQ ID NO: 22 - DNA, 50 bp, Artificial Sequence
  SPE Primer Pool
aatgtacagtattgcgttttggagaggcacgagaacacacatctattctg

SEQ ID NO: 23 - DNA, 50 bp, Artificial Sequence
  SPE Primer Pool
aatgtacagtattgcgttttgttctcttcagaagttccttcgtcatcctt

SEQ ID NO: 24 - DNA, 48 bp, Artificial Sequence
  SPE Primer Pool
aatgtacagtattgcgttttgtgatgacatgccccatcactaaaacac

SEQ ID NO: 25 - DNA, 56 bp, Artificial Sequence
  SPE Primer Pool
aatgtacagtattgcgttttgtgatagagacatgatgtaaccgtgggaatttcttc

SEQ ID NO: 26 - DNA, 55 bp, Artificial Sequence
  SPE Primer Pool
aatgtacagtattgcgttttgcgttctaagagagtgacagaaaggtaaagaggag

SEQ ID NO: 27 - DNA, 57 bp, Artificial Sequence
  SPE Primer Pool
aatgtacagtattgcgttttgatcacaaagtatctttttctgtggcttagaaatctt

SEQ ID NO: 28 - DNA, 57 bp, Artificial Sequence
  SPE Primer Pool
aatgtacagtattgcgttttgtcaaatgttagctcatttttgttaatggtggctttt

SEQ ID NO: 29 - DNA, 57 bp, Artificial Sequence
  SPE Primer Pool
aatgtacagtattgcgttttgtgtcacattataaagattcaggcaatgtttgttagt

SEQ ID NO: 30 - DNA, 57 bp, Artificial Sequence
  SPE Primer Pool
aatgtacagtattgcgttttgagtttgtatgcaacatttctaaagttacctacttgt

SEQ ID NO: 31 - DNA, 57 bp, Artificial Sequence
  SPE Primer Pool
aatgtacagtattgcgttttgaaaatctgttttccaataaattctcagatccaggaa

SEQ ID NO: 32 - DNA, 57 bp, Artificial Sequence
  SPE Primer Pool
aatgtacagtattgcgttttgcgacccagttaccatagcaatttagtgaaataacta

SEQ ID NO: 33 - DNA, 57 bp, Artificial Sequence
  SPE Primer Pool
aatgtacagtattgcgttttgagaggcgctatgtgtattattatagctacctgttaa

SEQ ID NO: 34 - DNA, 55 bp, Artificial Sequence
  SPE Primer Pool
aatgtacagtattgcgttttgcgtttttgacagtttgacagttaaaggcatttcc

SEQ ID NO: 35 - DNA, 57 bp, Artificial Sequence
  SPE Primer Pool
aatgtacagtattgcgttttgctgtccttattttggatatttctcccaatgaaagta

SEQ ID NO: 36 - DNA, 57 bp, Artificial Sequence
  SPE Primer Pool
aatgtacagtattgcgttttggactttttgcaaatgtttaacataggtgacagattt

SEQ ID NO: 37 - DNA, 57 bp, Artificial Sequence
  SPE Primer Pool
aatgtacagtattgcgttttgaagtagaaaatggaagtctatgtgatcaagaaatcg

SEQ ID NO: 38 - DNA, 55 bp, Artificial Sequence
  SPE Primer Pool
aatgtacagtattgcgttttgggcctcttaaagatcatgtttgttacagtgctta

SEQ ID NO: 39 - DNA, 57 bp, Artificial Sequence
  SPE Primer Pool
aatgtacagtattgcgttttgacaagattggtcaggaaaagagaattgttcctataa

SEQ ID NO: 40 - DNA, 57 bp, Artificial Sequence
  SPE Primer Pool
aatgtacagtattgcgttttgagaccctgtctcaaaagtaaaaagtaagttaacatg

SEQ ID NO: 41 - DNA, 56 bp, Artificial Sequence
  SPE Primer Pool
aatgtacagtattgcgttttgtcagtgtcttccaaatccttatgtatagcagcaat

SEQ ID NO: 42 - DNA, 48 bp, Artificial Sequence
  SPE Primer Pool
aatgtacagtattgcgttttgagggtcgaggaagccagtttacatcaa

SEQ ID NO: 43 - DNA, 57 bp, Artificial Sequence
  SPE Primer Pool
aatgtacagtattgcgttttgaacaaaaagatattttcaatatttctgcgcaggttt

SEQ ID NO: 44 - DNA, 57 bp, Artificial Sequence
  SPE Primer Pool
aatgtacagtattgcgttttggtctcgacttgaattgcaaaaagatgttagaaaagc

SEQ ID NO: 45 - DNA, 57 bp, Artificial Sequence
  SPE Primer Pool
aatgtacagtattgcgttttgaaaatgttggcagtcataacatttgaaactaatgga

SEQ ID NO: 46 - DNA, 55 bp, Artificial Sequence
  SPE Primer Pool
aatgtacagtattgcgttttgagcctcaaacaggttggttttaaatttgaagtct

SEQ ID NO: 47 - DNA, 56 bp, Artificial Sequence
  SPE Primer Pool
aatgtacagtattgcgttttgcctctgtgtgtatgttttaactacaaagcgaaaca

SEQ ID NO: 48 - DNA, 57 bp, Artificial Sequence
  SPE Primer Pool
aatgtacagtattgcgttttggattcacctggtaatgaggaaaacagctttaaaatc

SEQ ID NO: 49 - DNA, 56 bp, Artificial Sequence
  SPE Primer Pool
aatgtacagtattgcgttttgagatctgctgaaaagaaatttgttaaagcacaatt

SEQ ID NO: 50 - DNA, 45 bp, Artificial Sequence
  SPE Primer Pool
aatgtacagtattgcgttttgcggcatcccctacatcgagacctc

SEQ ID NO: 51 - DNA, 46 bp, Artificial Sequence
  SPE Primer Pool
aatgtacagtattgcgttttgcagggagcagatcaaacgggtgaag

SEQ ID NO: 52 - DNA, 51 bp, Artificial Sequence
  SPE Primer Pool
aatgtacagtattgcgttttgcaagtcttttgaggacatccaccagtacag

SEQ ID NO: 53 - DNA, 46 bp, Artificial Sequence
  SPE Primer Pool
aatgtacagtattgcgttttgacgtgcctgttggacatcctggata

SEQ ID NO: 54 - DNA, 50 bp, Artificial Sequence
  SPE Primer Pool
aatgtacagtattgcgttttgcctgtactggtggatgtcctcaaaagact

SEQ ID NO: 55 - DNA, 49 bp, Artificial Sequence
  SPE Primer Pool
aatgtacagtattgcgttttgccctgaggagcgatgacggaatataagc

SEQ ID NO: 56 - DNA, 52 bp, Artificial Sequence
  SPE Primer Pool
aatgtacagtattgcgttttggtcgtattcgtccacaaaatggttctggatc

SEQ ID NO: 57 - DNA, 50 bp, Artificial Sequence
  SPE Primer Pool
aatgtacagtattgcgttttgtgactggcaattgtgtcaacaggtgaaaa

SEQ ID NO: 58 - DNA, 46 bp, Artificial Sequence
  SPE Primer Pool
aatgtacagtattgcgttttgcgccagctggagtttggtcatgttt

SEQ ID NO: 59 - DNA, 57 bp, Artificial Sequence
  SPE Primer Pool
aatgtacagtattgcgttttgaatccctctcatcacaatttcattccacaatagttt

SEQ ID NO: 60 - DNA, 56 bp, Artificial Sequence
  SPE Primer Pool
aatgtacagtattgcgttttgtcaacaacaaagagaatcatgaaatcaaccctagc

SEQ ID NO: 61 - DNA, 46 bp, Artificial Sequence
  SPE Primer Pool
aatgtacagtattgcgttttggatatggagccagcgtgttccgatt

SEQ ID NO: 62 - DNA, 43 bp, Artificial Sequence
  SPE Primer Pool
aatgtacagtattgcgttttgggcgcggaaagtcctcactctc

SEQ ID NO: 63 - DNA, 48 bp, Artificial Sequence
  SPE Primer Pool
aatgtacagtattgcgttttgtatggtgaggttcggcgtgtttaaacg

SEQ ID NO: 64 - DNA, 48 bp, Artificial Sequence
  SPE Primer Pool
aatgtacagtattgcgttttgtggtgacaaagttagaagggtccatgg

SEQ ID NO: 65 - DNA, 50 bp, Artificial Sequence
  SPE Primer Pool
aatgtacagtattgcgttttgcttctttaccaccccagatacgacgacta

SEQ ID NO: 66 - DNA, 45 bp, Artificial Sequence
  SPE Primer Pool
aatgtacagtattgcgttttgcgctcgtggtggtagtcgtcgtat

SEQ ID NO: 67 - DNA, 47 bp, Artificial Sequence
  SPE Primer Pool
aatgtacagtattgcgttttgccaggaggccctttctgtttacaacc

SEQ ID NO: 68 - DNA, 53 bp, Artificial Sequence
  SPE Primer Pool
aatgtacagtattgcgttttgcccacaagcccaaaatattctactcactttgc

SEQ ID NO: 69 - DNA, 49 bp, Artificial Sequence
  SPE Primer Pool
aatgtacagtattgcgttttgatcgcctgcatcaaggaaaaggtaatgg

SEQ ID NO: 70 - DNA, 50 bp, Artificial Sequence
  SPE Primer Pool
aatgtacagtattgcgttttgcgcgtaaggatagcaactgaggttatcac

SEQ ID NO: 71 - DNA, 47 bp, Artificial Sequence
  SPE Primer Pool
aatgtacagtattgcgttttgcgacctgacgtaaccccttgcttatc

SEQ ID NO: 72 - DNA, 51 bp, Artificial Sequence
  SPE Primer Pool
aatgtacagtattgcgttttgggaaatgctctcacgtagtctctcatgtct

SEQ ID NO: 73 - DNA, 51 bp, Artificial Sequence
  SPE Primer Pool
aatgtacagtattgcgttttggtcataacccgaagaacaatgttgccacta

SEQ ID NO: 74 - DNA, 50 bp, Artificial Sequence
  SPE Primer Pool
aatgtacagtattgcgttttggtcagctcaggataaagcacggatggata

SEQ ID NO: 75 - DNA, 57 bp, Artificial Sequence
  SPE Primer Pool
aatgtacagtattgcgttttgctcaggataaaagcttccttcttaacaagtttttcc

SEQ ID NO: 76 - DNA, 53 bp, Artificial Sequence
  SPE Primer Pool
aatgtacagtattgcgttttgagagattgttcccttgcattgacctctttttc

SEQ ID NO: 77 - DNA, 50 bp, Artificial Sequence
  SPE Primer Pool
aatgtacagtattgcgttttgcccctcacctttggaatttacagtctgaa

SEQ ID NO: 78 - DNA, 56 bp, Artificial Sequence
  SPE Primer Pool
aatgtacagtattgcgttttgtaggttcttcaggtctctacactctcctttaaact

SEQ ID NO: 79 - DNA, 52 bp, Artificial Sequence
  SPE Primer Pool
aatgtacagtattgcgttttggagaaggagtgcaatgccaagattatgatcc

SEQ ID NO: 80 - DNA, 50 bp, Artificial Sequence
  SPE Primer Pool
aatgtacagtattgcgttttggacgttctccattgtattggcagtaacca

SEQ ID NO: 81 - DNA, 57 bp, Artificial Sequence
  SPE Primer Pool
aatgtacagtattgcgttttgcacatctcacaggctctaaaggaattctatatccta

SEQ ID NO: 82 - DNA, 52 bp, Artificial Sequence
  SPE Primer Pool
aatgtacagtattgcgttttggaggcaagaggtgagtagtaccaatactgtc

SEQ ID NO: 83 - DNA, 47 bp, Artificial Sequence
  SPE Primer Pool
aatgtacagtattgcgttttggagcccctccgcttacttgtaatctg

SEQ ID NO: 84 - DNA, 57 bp, Artificial Sequence
  SPE Primer Pool
aatgtacagtattgcgttttgccagtaaaacgtattgagaaaaaggtaaaagcgtta

SEQ ID NO: 85 - DNA, 57 bp, Artificial Sequence
  SPE Primer Pool
aatgtacagtattgcgttttggctcagaataaatcgtaacaatctcaaagtgcattt

SEQ ID NO: 86 - DNA, 49 bp, Artificial Sequence
  SPE Primer Pool
aatgtacagtattgcgttttgtgaggtgtccacagggctcaatctttac

SEQ ID NO: 87 - DNA, 57 bp, Artificial Sequence
  SPE Primer Pool
aatgtacagtattgcgttttgccccttgtatcagtaaaggctatataataccgaatt

SEQ ID NO: 88 - DNA, 55 bp, Artificial Sequence
  SPE Primer Pool
aatgtacagtattgcgttttgtcatgaagagagtatcatcagctcgttcatcatc

SEQ ID NO: 89 - DNA, 53 bp, Artificial Sequence
  SPE Primer Pool
aatgtacagtattgcgttttgtgtcctttctgccgatgtgaaattaaaggtac

SEQ ID NO: 90 - DNA, 47 bp, Artificial Sequence
  SPE Primer Pool
aatgtacagtattgcgttttgtcgccccaaataatttcctgcgaaca

SEQ ID NO: 91 - DNA, 53 bp, Artificial Sequence
  SPE Primer Pool
aatgtacagtattgcgttttgctcatacctccattccaagctttcattgtctc

SEQ ID NO: 92 - DNA, 52 bp, Artificial Sequence
  SPE Primer Pool
aatgtacagtattgcgttttgcctgcccttatttttaacagcaggaacgaat

SEQ ID NO: 93 - DNA, 48 bp, Artificial Sequence
  SPE Primer pool
aatgtacagtattgcgttttgtcgatagcgaaagtcctctttggtcag

SEQ ID NO: 94 - DNA, 57 bp, Artificial Sequence
  SPE Primer Pool
aatgtacagtattgcgttttggttaaagaccaaccactaactaagagactttccaag

SEQ ID NO: 95 - DNA, 52 bp, Artificial Sequence
  SPE Primer Pool
aatgtacagtattgcgttttgaaacctcttccagtaccttcttcatggttct

SEQ ID NO: 96 - DNA, 51 bp, Artificial Sequence
  SPE Primer Pool
aatgtacagtattgcgttttgtttccaggtgatgtgctctatgaactcctt

SEQ ID NO: 97 - DNA, 45 bp, Artificial Sequence
  SPE Primer Pool
aatgtacagtattgcgttttgggagcggtgcaacagttcaatggt

SEQ ID NO: 98 - DNA, 48 bp, Artificial Sequence
  SPE Primer Pool
aatgtacagtattgcgttttgcatccgtggataatgtgcaccataacc

SEQ ID NO: 99 - DNA, 47 bp, Artificial Sequence
  SPE Primer Pool
aatgtacagtattgcgttttgtcggagagcctggactgtttgaaatc

SEQ ID NO: 100 - DNA, 46 bp, Artificial Sequence
  SPE Primer Pool
aatgtacagtattgcgttttgaagccaggtcttcccgatgagagag

SEQ ID NO: 101 - DNA, 46 bp, Artificial Sequence
  SPE Primer Pool
aatgtacagtattgcgttttgggcactccgtggatttcaaacagtc

SEQ ID NO: 102 - DNA, 53 bp, Artificial Sequence
  SPE Primer Pool
aatgtacagtattgcgttttgcagatatctgctgcccttttaccttatggttt

SEQ ID NO: 103 - DNA, 54 bp, Artificial Sequence
  SPE Primer Pool
aatgtacagtattgcgttttgtgtagactgctttgggattacgtctatcagttg

SEQ ID NO: 104 - DNA, 52 bp, Artificial Sequence
  SPE Primer Pool
aatgtacagtattgcgttttgggaaaggagaaaaaggaagtgctacctgaac

SEQ ID NO: 105 - DNA, 51 bp, Artificial Sequence
  SPE Primer Pool
aatgtacagtattgcgttttgtttttctcccttcctcctttgaacaaacag

SEQ ID NO: 106 - DNA, 55 bp, Artificial Sequence
  SPE Primer Pool
aatgtacagtattgcgttttgacagctttaggaaaatggaatctcttacctcctc

SEQ ID NO: 107 - DNA, 48 bp, Artificial Sequence
  SPE Primer Pool
aatgtacagtattgcgttttggggtgttatggtcgcgttggatttctg

SEQ ID NO: 108 - DNA, 45 bp, Artificial Sequence
  SPE Primer Pool
aatgtacagtattgcgttttggctacggcgtgcaactcacagaac

SEQ ID NO: 109 - DNA, 44 bp, Artificial Sequence
  SPE Primer Pool
aatgtacagtattgcgttttgaccgacctcttccagcgctactt

SEQ ID NO: 110 - DNA, 44 bp, Artificial Sequence
  SPE Primer Pool
aatgtacagtattgcgttttgcgggcagggcttacttaccttgg

SEQ ID NO: 111 - DNA, 49 bp, Artificial Sequence
  SPE Primer Pool
aatgtacagtattgcgttttgtagctactgcctgccttcgaagaacgat

SEQ ID NO: 112 - DNA, 54 bp, Artificial Sequence
  SPE Primer Pool
aatgtacagtattgcgttttgtgtgggtggaaaaagatgtggttaagaaacaac

SEQ ID NO: 113 - DNA, 50 bp, Artificial Sequence
  SPE Primer Pool
aatgtacagtattgcgttttgcccccatatagcttaatctgatgggcatc

SEQ ID NO: 114 - DNA, 51 bp, Artificial Sequence
  SPE Primer Pool
aatgtacagtattgcgttttggaaagagcatcaggaacaagccttgagtac

SEQ ID NO: 115 - DNA, 51 bp, Artificial Sequence
  SPE Primer Pool
aatgtacagtattgcgttttgttgagatgcctgacaacctttacacctttg

SEQ ID NO: 116 - DNA, 49 bp, Artificial Sequence
  SPE Primer Pool
aatgtacagtattgcgttttgctctagggctgagggaatatgcatctct

SEQ ID NO: 117 - DNA, 49 bp, Artificial Sequence
  SPE Primer Pool
aatgtacagtattgcgttttgcgtacccagaagacaatggcctagctat

SEQ ID NO: 118 - DNA, 45 bp, Artificial Sequence
  SPE Primer Pool
aatgtacagtattgcgttttggggcagcacagattcccttaacca

SEQ ID NO: 119 - DNA, 49 bp, Artificial Sequence
  SPE Primer Pool
aatgtacagtattgcgttttgccataccttggctatcccctgaaagttg

SEQ ID NO: 120 - DNA, 46 bp, Artificial Sequence
  SPE Primer Pool
aatgtacagtattgcgttttggccctgatgctcatggagtgttcct

SEQ ID NO: 121 - DNA, 44 bp, Artificial Sequence
  SPE Primer Pool
aatgtacagtattgcgttttgcctggtggttgggagacgactac

SEQ ID NO: 122 - DNA, 49 bp, Artificial Sequence
  SPE Primer Pool
aatgtacagtattgcgttttgtgctgacaggacacagaacaagatacct

SEQ ID NO: 123 - DNA, 52 bp, Artificial Sequence
  SPE Primer Pool
aatgtacagtattgcgttttgggtacaggtatcttgttctgtgtcctgtcag

SEQ ID NO: 124 - DNA, 43 bp, Artificial Sequence
  SPE Primer Pool
aatgtacagtattgcgttttggagtcccgggctcgattcacag

SEQ ID NO: 125 - DNA, 50 bp, Artificial Sequence
  SPE Primer Pool
aatgtacagtattgcgttttgctggtcagagaggtgtgtactgattgtct

SEQ ID NO: 126 - DNA, 57 bp, Artificial Sequence
  SPE Primer Pool
aatgtacagtattgcgttttgaggaaagatcaattacattcacaagttcacacttct

SEQ ID NO: 127 - DNA, 55 bp, Artificial Sequence
  SPE Primer Pool
aatgtacagtattgcgttttgctgcacagttcagaggatatttaagctcaatgac

SEQ ID NO: 128 - DNA, 49 bp, Artificial Sequence
  SPE Primer Pool
aatgtacagtattgcgttttgcacagaccgtcatgcatttctgacactc

SEQ ID NO: 129 - DNA, 47 bp, Artificial Sequence
  SPE Primer Pool
aatgtacagtattgcgttttgaggctggtacctgctcttcttcaatc

SEQ ID NO: 130 - DNA, 53 bp, Artificial Sequence
  SPE Primer Pool
aatgtacagtattgcgttttgcgaaatcaaacagttgtctatcagagcctgtc

SEQ ID NO: 131 - DNA, 55 bp, Artificial Sequence
  SPE Primer Pool
aatgtacagtattgcgttttgacaaaagaaaagaagtcatgtctgtatgtggaaa

SEQ ID NO: 132 - DNA, 57 bp, Artificial Sequence
  SPE Primer Pool
aatgtacagtattgcgttttgtccaggataatacacatcacagtaaataacactctg

SEQ ID NO: 133 - DNA, 55 bp, Artificial Sequence
  SPE Primer Pool
aatgtacagtattgcgttttgcatcctctttgtcatcaagctacagtctttttga

SEQ ID NO: 134 - DNA, 51 bp, Artificial Sequence
  SPE Primer Pool
aatgtacagtattgcgttttgctcccatttttgtgcatctttgttgctgtc

SEQ ID NO: 135 - DNA, 55 bp, Artificial Sequence
  SPE Primer Pool
aatgtacagtattgcgttttgcagaactgcctattcctaactgactcatcatttc

SEQ ID NO: 136 - DNA, 54 bp, Artificial Sequence
  SPE Primer Pool
aatgtacagtattgcgttttggaattctgtttcatcgctgagtgacactctttt

SEQ ID NO: 137 - DNA, 57 bp, Artificial Sequence
  SPE Primer Pool
aatgtacagtattgcgttttgtttttacctttgcttttacctttttgtacttgtgac

SEQ ID NO: 138 - DNA, 53 bp, Artificial Sequence
  SPE Primer Pool
aatgtacagtattgcgttttgagaaggagtctggaatagaaaggctaacagaa

SEQ ID NO: 139 - DNA, 49 bp, Artificial Sequence
  SPE Primer Pool
aatgtacagtattgcgttttgcacaagatgtgccaagggaattgtatgc

SEQ ID NO: 140 - DNA, 57 bp, Artificial Sequence
  SPE Primer Pool
aatgtacagtattgcgttttgaagagtcaataggtcagagagttttatgttcttcca

SEQ ID NO: 141 - DNA, 52 bp, Artificial Sequence
  SPE Primer Pool
aatgtacagtattgcgttttgactgatcttctcaaagtcgtcatccttcagt

SEQ ID NO: 142 - DNA, 57 bp, Artificial Sequence
  SPE Primer Pool
aatgtacagtattgcgttttgaccctgagaaataatccaattacctgttaatcaagg

SEQ ID NO: 143 - DNA, 57 bp, Artificial Sequence
  SPE Primer Pool
aatgtacagtattgcgttttgaaaaggtattgagtaaaatcagtcttccttctaccc

SEQ ID NO: 144 - DNA, 55 bp, Artificial Sequence
  SPE Primer Pool
aatgtacagtattgcgttttgccttcctccctctttctttcataaaacctctctt

SEQ ID NO: 145 - DNA, 45 bp, Artificial Sequence
  SPE Primer Pool
aatgtacagtattgcgttttggccagagccacccaactcttaagg

SEQ ID NO: 146 - DNA, 57 bp, Artificial Sequence
  SPE Primer Pool
aatgtacagtattgcgttttgtggaagaggaatttaataacgaacgttttaagagga

SEQ ID NO: 147 - DNA, 47 bp, Artificial Sequence
  SPE Primer Pool
aatgtacagtattgcgttttggcatctactgccgaggatgttccaag

SEQ ID NO: 148 - DNA, 46 bp, Artificial Sequence
  SPE Primer Pool
aatgtacagtattgcgttttgcacagtgagctcaagtgcgacatca

SEQ ID NO: 149 - DNA, 43 bp, Artificial Sequence
  SPE Primer Pool
aatgtacagtattgcgttttgccgactggccatctcctcgtag

SEQ ID NO: 150 - DNA, 45 bp, Artificial Sequence
  SPE Primer Pool
aatgtacagtattgcgttttggtaccagcgcgactacgaggagat

SEQ ID NO: 151 - DNA, 57 bp, Artificial Sequence
  SPE Primer Pool
aatgtacagtattgcgttttgtcttttctgtcaaatggagatgatctcttctgactc

SEQ ID NO: 152 - DNA, 48 bp, Artificial Sequence
  SPE Primer Pool
aatgtacagtattgcgttttggggagcccatcatctgcaaaaacatcc

SEQ ID NO: 153 - DNA, 52 bp, Artificial Sequence
  SPE Primer Pool
aatgtacagtattgcgttttgaagctgaagaagatgtggaaaagtcccaatg

SEQ ID NO: 154 - DNA, 47 bp, Artificial Sequence
  SPE Primer Pool
aatgtacagtattgcgttttggcgtgggatgtttttgcagatgatgg

SEQ ID NO: 155 - DNA, 44 bp, Artificial Sequence
  SPE Primer Pool
aatgtacagtattgcgttttgcgacgctgaggacgctatggatg

SEQ ID NO: 156 - DNA, 43 bp, Artificial Sequence
  SPE Primer Pool
aatgtacagtattgcgttttggctgaggcgcgtcttcgagaag

SEQ ID NO: 157 - DNA, 44 bp, Artificial Sequence
  SPE Primer Pool
aatgtacagtattgcgttttggcgcttgtcgtgaaagcgaacga

SEQ ID NO: 158 - DNA, 42 bp, Artificial Sequence
  SPE Primer Pool
aatgtacagtattgcgttttggctgcccgcccagttgttact

SEQ ID NO: 159 - DNA, 51 bp, Artificial Sequence
  SPE Primer Pool
aatgtacagtattgcgttttgagactctggactgatgaagcaattctgagt

SEQ ID NO: 160 - DNA, 48 bp, Artificial Sequence
  SPE Primer Pool
aatgtacagtattgcgttttgtcaccggtgacaccttaaaaccaaagc

SEQ ID NO: 161 - DNA, 50 bp, Artificial Sequence
  SPE Primer Pool
aatgtacagtattgcgttttgggctcctttgtacctcctccatcttgatc

SEQ ID NO: 162 - DNA, 57 bp, Artificial Sequence
  SPE Primer Pool
aatgtacagtattgcgttttggtcagttgtctaacaataacaaagatctgctcttgg

SEQ ID NO: 163 - DNA, 49 bp, Artificial Sequence
  SPE Primer Pool
aatgtacagtattgcgttttgggtgggcagcaagaaaaagtccagtaaa

SEQ ID NO: 164 - DNA, 49 bp, Artificial Sequence
  SPE Primer Pool
aatgtacagtattgcgttttggccaaggctttctctggcatgatctttt

SEQ ID NO: 165 - DNA, 55 bp, Artificial Sequence
  SPE Primer Pool
aatgtacagtattgcgttttgggataactttctcagcatttccaccagtttcaag

SEQ ID NO: 166 - DNA, 57 bp, Artificial Sequence
  SPE Primer Pool
aatgtacagtattgcgttttgtgtccctaagttgagtaaaatgatagagaatgagtc

SEQ ID NO: 167 - DNA, 50 bp, Artificial Sequence
  SPE Primer Pool
aatgtacagtattgcgttttggctgccagaaatccagcatccaaaatttg

SEQ ID NO: 168 - DNA, 51 bp, Artificial Sequence
  SPE Primer Pool
aatgtacagtattgcgttttggtcgctttcttttcttagtgccaggaaact

SEQ ID NO: 169 - DNA, 50 bp, Artificial Sequence
  SPE Primer Pool
aatgtacagtattgcgttttgacagtcgagacgattcatgagggaacttc

SEQ ID NO: 170 - DNA, 48 bp, Artificial Sequence
  SPE Primer Pool
aatgtacagtattgcgttttgggaaagctcggcgtgttggataagaag

SEQ ID NO: 171 - DNA, 48 bp, Artificial Sequence
  SPE Primer Pool
aatgtacagtattgcgttttgacgccacaagtgactgaaagttggaag

SEQ ID NO: 172 - DNA, 50 bp, Artificial Sequence
  SPE Primer Pool
aatgtacagtattgcgttttgtgatgggctggagatttggcatagttttc

SEQ ID NO: 173 - DNA, 50 bp, Artificial Sequence
  SPE Primer Pool
aatgtacagtattgcgttttgctatgcacccactttcaacacagttaggt

SEQ ID NO: 174 - DNA, 47 bp, Artificial Sequence
  SPE Primer Pool
aatgtacagtattgcgttttggcttggtcagaagtgctgttgttgtc

SEQ ID NO: 175 - DNA, 47 bp, Artificial Sequence
  SPE Primer Pool
aatgtacagtattgcgttttgcgtgggccagaaagttgtccacaatg

SEQ ID NO: 176 - DNA, 52 bp, Artificial Sequence
  SPE Primer Pool
aatgtacagtattgcgttttggggatatggattctcgtggtagaaggtgtaa

SEQ ID NO: 177 - DNA, 54 bp, Artificial Sequence
  SPE Primer Pool
aatgtacagtattgcgttttgctaatcaccaagttccaagtgttcagaatctcc

SEQ ID NO: 178 - DNA, 55 bp, Artificial Sequence
  SPE Primer Pool
aatgtacagtattgcgttttgaccgtaataaccaaggttcatcataggcattgat

SEQ ID NO: 179 - DNA, 49 bp, Artificial Sequence
  SPE Primer Tool
aatgtacagtattgcgttttgtcccagtggaagttactatgcaccctat

SEQ ID NO: 180 - DNA, 54 bp, Artificial Sequence
  SPE Primer Pool
aatgtacagtattgcgttttgtgcttatgcttgtgtttgtgtttcctcttatgg

SEQ ID NO: 181 - DNA, 54 bp, Artificial Sequence
  SPE Primer Pool
aatgtacagtattgcgttttggcttctgtttctccttatgcttgttcttctcac

SEQ ID NO: 182 - DNA, 47 bp, Artificial Sequence
  SPE Primer Pool
aatgtacagtattgcgttttgcctgagtggtctttttgcaggcaaag

SEQ ID NO: 183 - DNA, 47 bp, Artificial Sequence
  SPE Primer Pool
aatgtacagtattgcgttttgccggccacaaagcttctaagaacaac

SEQ ID NO: 184 - DNA, 47 bp, Artificial Sequence
  SPE Primer Pool
aatgtacagtattgcgttttggcggttcatcttgaaggcttggatgt

SEQ ID NO: 185 - DNA, 51 bp, Artificial Sequence
  SPE Primer Pool
aatgtacagtattgcgttttgttcagtgaaatgaacccttcgaatgacaag

SEQ ID NO: 186 - DNA, 48 bp, Artificial Sequence
  SPE Primer Pool
aatgtacagtattgcgttttgctcctcctcctctttgcgtttcttgtc

SEQ ID NO: 187 - DNA, 51 bp, Artificial Sequence
  SPE Primer Pool
aatgtacagtattgcgttttggcagcagagaaacaaatgaaggacaaacag

SEQ ID NO: 188 - DNA, 52 bp, Artificial Sequence
  SPE Primer Pool
aatgtacagtattgcgttttgtaaggaggaggaagaagacaagaaacgcaaa

SEQ ID NO: 189 - DNA, 50 bp, Artificial Sequence
  SPE Primer Pool
aatgtacagtattgcgttttgtaaggcaggtctgtgagcacaaaatttgg

SEQ ID NO: 190 - DNA, 45 bp, Artificial Sequence
  SPE Primer Pool
aatgtacagtattgcgttttgtggagctgaccagtgacaatgacc

SEQ ID NO: 191 - DNA, 46 bp, Artificial Sequence
  SPE Primer Pool
aatgtacagtattgcgttttgggccaagaagtcggtggacaagaac

SEQ ID NO: 192 - DNA, 43 bp, Artificial Sequence
  SPE Primer Pool
aatgtacagtattgcgttttggcgcaggcggtcattgtcactg

SEQ ID NO: 193 - DNA, 48 bp, Artificial Sequence
  SPE Primer Pool
aatgtacagtattgcgttttgttgctgttcttgtccaccgacttcttg

SEQ ID NO: 194 - DNA, 42 bp, Artificial Sequence
  SPE Primer Pool
aatgtacagtattgcgttttggcagtgcgcgatctggaactg

SEQ ID NO: 195 - DNA, 42 bp, Artificial Sequence
  SPE Primer Pool
aatgtacagtattgcgttttgcggcggcgactttgactaccc

SEQ ID NO: 196 - DNA, 45 bp, Artificial Sequence
  SPE Primer Pool
aatgtacagtattgcgttttggagcacgagacgtccatcgacatc

SEQ ID NO: 197 - DNA, 43 bp, Artificial Sequence
  SPE Primer Pool
aatgtacagtattgcgttttgcggccaggaactcgtcgttgaa

SEQ ID NO: 198 - DNA, 45 bp, Artificial Sequence
  SPE Primer Pool
aatgtacagtattgcgttttggccatgccgggagaactctaactc

SEQ ID NO: 199 - DNA, 54 bp, Artificial Sequence
  SPE Primer Pool
aatgtacagtattgcgttttgtgtaaccctcctaagtgttcatacgttgtcttg

SEQ ID NO: 200 - DNA, 57 bp, Artificial Sequence
  SPE Primer Pool
aatgtacagtattgcgttttggtcttggtctctgttatatcttgagtctagaacagt

SEQ ID NO: 201 - DNA, 49 bp, Artificial Sequence
  SPE Primer Pool
aatgtacagtattgcgttttgcaggagaacatggaggcgagaagaaaat

SEQ ID NO: 202 - DNA, 48 bp, Artificial Sequence
  SPE Primer Pool
aatgtacagtattgcgttttggggaaagattggatgccgggaatcaac

SEQ ID NO: 203 - DNA, 46 bp, Artificial Sequence
  SPE Primer Pool
aatgtacagtattgcgttttgcggaggcttgattaggtaggaggtg

SEQ ID NO: 204 - DNA, 45 bp, Artificial Sequence
  SPE Primer Pool
aatgtacagtattgcgttttggcggcagctcaacgagaataaaca

SEQ ID NO: 205 - DNA, 45 bp, Artificial Sequence
  SPE Primer Pool
aatgtacagtattgcgttttggcccgcatccttactccgcttatc

SEQ ID NO: 206 - DNA, 49 bp, Artificial Sequence
  SPE Primer Pool
aatgtacagtattgcgttttggctggtttcaaggtaagtggactcttcc

SEQ ID NO: 207 - DNA, 47 bp, Artificial Sequence
  SPE Primer Pool
aatgtacagtattgcgttttggggaatgactgacggagaatcccaac

SEQ ID NO: 208 - DNA, 48 bp, Artificial Sequence
  SPE Primer Pool
aatgtacagtattgcgttttgctaagaccgagagcctgtaggagcttt

SEQ ID NO: 209 - DNA, 42 bp, Artificial Sequence
  SPE Primer Pool
aatgtacagtattgcgttttggccgggcttgtctggtcatct

SEQ ID NO: 210 - DNA, 51 bp, Artificial Sequence
  SPE Primer Pool
aatgtacagtattgcgttttgcagctcacctccaaaaaggcaaaattcttg

SEQ ID NO: 211 - DNA, 48 bp, Artificial Sequence
  SPE Primer Pool
aatgtacagtattgcgttttggcaggaggccatgatggatttcttcaa

SEQ ID NO: 212 - DNA, 53 bp, Artificial Sequence
  SPE Primer Pool
aatgtacagtattgcgttttgcatgagtgaaaggaaagaggaaatcccaatcc

SEQ ID NO: 213 - DNA, 56 bp, Artificial Sequence
  SPE Primer Pool
aatgtacagtattgcgttttgcctatcttccacagtacttacacaacttcctaagc

SEQ ID NO: 214 - DNA, 45 bp, Artificial Sequence
  SPE Primer Pool
aatgtacagtattgcgttttgctcgccgtagactgtccaggtttt

SEQ ID NO: 215 - DNA, 47 bp, Artificial Sequence
  SPE Primer
aatgtacagtattgcgttttgctcacctgatccgtgacgttgatgtc

SEQ ID NO: 216 - DNA, 44 bp, Artificial Sequence
  SPE Primer Pool
aatgtacagtattgcgttttggccctgatggactctcggctact

SEQ ID NO: 217 - DNA, 53 bp, Artificial Sequence
  SPE Primer Pool
aatgtacagtattgcgttttggagaaagatcaggaacacttgtcccctactag

SEQ ID NO: 218 - DNA, 48 bp, Artificial Sequence
  SPE Primer Pool
aatgtacagtattgcgttttggtcctccacgatctcctcatactcctc

SEQ ID NO: 219 - DNA, 47 bp, Artificial Sequence
  SPE Primer Pool
aatgtacagtattgcgttttgtcgatggacttgacaagcccgtactt

SEQ ID NO: 220 - DNA, 48 bp, Artificial Sequence
  SPE Primer Pool
aatgtacagtattgcgttttgctggacgacgaggagtatgaggagatc

SEQ ID NO: 221 - DNA, 46 bp, Artificial Sequence
  SPE Primer Pool
aatgtacagtattgcgttttgtaccagaagtcccggcggtgataag

SEQ ID NO: 222 - DNA, 49 bp, Artificial Sequence
  SPE Primer Pool
aatgtacagtattgcgttttggttcacctctgtgtttgactgccagaaa

SEQ ID NO: 223 - DNA, 57 bp, Artificial Sequence
  SPE Primer Pool
aatgtacagtattgcgttttgcaatgagtattctcttcatttcaggtcagttgattt

SEQ ID NO: 224 - DNA, 49 bp, Artificial Sequence
  SPE Primer Pool
aatgtacagtattgcgttttgggctgctttcttgaaggctattgggtat

SEQ ID NO: 225 - DNA, 53 bp, Artificial Sequence
  SPE Primer Pool
aatgtacagtattgcgttttgaggagactggaattctcgaataaggattaaca

SEQ ID NO: 226 - DNA, 57 bp, Artificial Sequence
  SPE Primer Pool
aatgtacagtattgcgttttggcatagttaaaacctgtgtttggttttgtaggtctt

SEQ ID NO: 227 - DNA, 48 bp, Artificial Sequence
  SPE Primer Pool
aatgtacagtattgcgttttgctctgtgttggcggatacccttccata

SEQ ID NO: 228 - DNA, 53 bp, Artificial Sequence
  SPE Primer Pool
aatgtacagtattgcgttttgggcattccttctttattgcccttcttaaaagc

SEQ ID NO: 229 - DNA, 50 bp, Artificial Sequence
  SPE Primer Pool
aatgtacagtattgcgttttggctgctggtctggctactatgatctctac

SEQ ID NO: 230 - DNA, 53 bp, Artificial Sequence
  SPE Primer Pool
aatgtacagtattgcgttttggcacacagcttttaagaagggcaataaagaag

SEQ ID NO: 231 - DNA, 57 bp, Artificial Sequence
  SPE Primer Pool
aatgtacagtattgcgttttgtgtatgtttaattctgtacatgagcatttcatcagt

SEQ ID NO: 232 - DNA, 57 bp, Artificial Sequence
  SPE Primer Pool
aatgtacagtattgcgttttgatttcataccttgcttaatgggtgtagataccaaaa

SEQ ID NO: 233 - DNA, 48 bp, Artificial Sequence
  SPE Primer Pool
aatgtacagtattgcgttttgttggcgtcaaatgtgccactatcactc

SEQ ID NO: 234 - DNA, 57 bp, Artificial Sequence
  SPE Primer Pool
aatgtacagtattgcgttttgttctctttcaagctatgatttaggcatagagaatcg

SEQ ID NO: 235 - DNA, 57 bp, Artificial Sequence
  SPE Primer Pool
aatgtacagtattgcgttttgctgcagttgtaggttataactatccatttgtctgaa

SEQ ID NO: 236 - DNA, 52 bp, Artificial Sequence
  SPE Primer Pool
aatgtacagtattgcgttttgccctaggtcagatcacccagtcagttaaaac

SEQ ID NO: 237 - DNA, 52 bp, Artificial Sequence
  SPE Primer Pool
aatgtacagtattgcgttttgtggttaaaggtcagcccacttaccagatatg

SEQ ID NO: 238 - DNA, 49 bp, Artificial Sequence
  SPE Primer Pool
aatgtacagtattgcgttttggggtatgctccccatttagaggataagg

SEQ ID NO: 239 - DNA, 50 bp, Artificial Sequence
  SPE Primer Pool
aatgtacagtattgcgttttgacgtcagatctacagcgaacacaactact

SEQ ID NO: 240 - DNA, 50 bp, Artificial Sequence
  SPE Primer Pool
aatgtacagtattgcgttttgagtggtgccagactcacattcagttctaa

SEQ ID NO: 241 - DNA, 54 bp, Artificial Sequence
  SPE Primer Pool
aatgtacagtattgcgttttgcttggccagttcctttctctaatgtatcatctc

SEQ ID NO: 242 - DNA, 56 bp, Artificial Sequence
  SPE Primer Pool
aatgtacagtattgcgttttgaagttttcttgtctagtatcactttccctcatagg

SEQ ID NO: 243 - DNA, 49 bp, Artificial Sequence
  SPE Primer Pool
aatgtacagtattgcgttttggggctcaacagatggtatgtgttctctg

SEQ ID NO: 244 - DNA, 54 bp, Artificial Sequence
  SPE Primer Pool
aatgtacagtattgcgttttggctctcgtttctaacagttctttgcattggata

SEQ ID NO: 245 - DNA, 50 bp, Artificial Sequence
  SPE Primer Pool
aatgtacagtattgcgttttggaggtgaccttcaaagtcagaggctgtat

SEQ ID NO: 246 - DNA, 49 bp, Artificial Sequence
  SPE Primer Pool
aatgtacagtattgcgttttggagcaaccatcccatctgtccttgtaac

SEQ ID NO: 247 - DNA, 50 bp, Artificial Sequence
  SPE Primer Pool
aatgtacagtattgcgttttgggacaaggatgagaaacccaattggaacc

SEQ ID NO: 248 - DNA, 46 bp, Artificial Sequence
  SPE Primer Pool
aatgtacagtattgcgttttgcggtccgccaaaagatcccagattc

SEQ ID NO: 249 - DNA, 46 bp, Artificial Sequence
  SPE Primer Pool
aatgtacagtattgcgttttgggaggccactaacccacttgtgatg

SEQ ID NO: 250 - DNA, 54 bp, Artificial Sequence
  SPE Primer Pool
aatgtacagtattgcgttttgtccagtttcctagaggatgtaatgggatttgtc

SEQ ID NO: 251 - DNA, 52 bp, Artificial Sequence
  SPE Primer Pool
aatgtacagtattgcgttttgtcacatttggagatgagaaacgaggtgttct

SEQ ID NO: 252 - DNA, 48 bp, Artificial Sequence
  SPE Primer Pool
aatgtacagtattgcgttttgcccttggcctgtaacattgctctgatc

SEQ ID NO: 253 - DNA, 52 bp, Artificial Sequence
  SPE Primer Pool
aatgtacagtattgcgttttgcacctcgtttctcatctccaaatgtgatctc

SEQ ID NO: 254 - DNA, 47 bp, Artificial Sequence
  SPE Primer Pool
aatgtacagtattgcgttttgccagtagctttcctgttctcggcatt

SEQ ID NO: 255 - DNA, 50 bp, Artificial Sequence
  SPE Primer Pool
aatgtacagtattgcgttttggcagcgtcaagaatgagaagacttttgtg

SEQ ID NO: 256 - DNA, 47 bp, Artificial Sequence
  SPE Primer Pool
aatgtacagtattgcgttttgttgcccttctggaaattaccccgaga

SEQ ID NO: 257 - DNA, 55 bp, Artificial Sequence
  SPE Primer Pool
aatgtacagtattgcgttttgagttccaccagctttaattattcctctagctctc

SEQ ID NO: 258 - DNA, 56 bp, Artificial Sequence
  SPE Primer Pool
aatgtacagtattgcgttttggtttcccatggccataatttattatctcaccacaa

SEQ ID NO: 259 - DNA, 48 bp, Artificial Sequence
  SPE Primer Pool
aatgtacagtattgcgttttggtcacgatgactgtattggaccctcaa

SEQ ID NO: 260 - DNA, 56 bp, Artificial Sequence
  SPE Primer Pool
aatgtacagtattgcgttttgtccagacctttgctttagattggcaattattactg

SEQ ID NO: 261 - DNA, 51 bp, Artificial Sequence
  SPE Primer Pool
aatgtacagtattgcgttttgccctaacaacacagaagcaaagcgttcttt

SEQ ID NO: 262 - DNA, 45 bp, Artificial Sequence
  SPE Primer Pool
aatgtacagtattgcgttttgcgccctcctaccacctgtactacg

SEQ ID NO: 263 - DNA, 46 bp, Artificial Sequence
  SPE Primer Pool
aatgtacagtattgcgttttgactatccaggcgccttcacctactc

SEQ ID NO: 264 - DNA, 47 bp, Artificial Sequence
  SPE Primer Pool
aatgtacagtattgcgttttgctcctaggcggtatcatcctgggtag

SEQ ID NO: 265 - DNA, 51 bp, Artificial Sequence
  SPE Primer Pool
aatgtacagtattgcgttttgtctgattctcttcagatacaaggcagatcc

SEQ ID NO: 266 - DNA, 55 bp, Artificial Sequence
  SPE Primer Pool
aatgtacagtattgcgttttggcagatacttggacttgagtaggcttattaaacc

SEQ ID NO: 267 - DNA, 57 bp, Artificial Sequence
  SPE Primer Pool
aatgtacagtattgcgttttggcggctctataaagaattgtccttattttcgaactt

SEQ ID NO: 268 - DNA, 48 bp, Artificial Sequence
  SPE Primer Pool
aatgtacagtattgcgttttggttcgaggcctttctctgagcatcaag

SEQ ID NO: 269 - DNA, 51 bp, Artificial Sequence
  SPE Primer Pool
aatgtacagtattgcgttttgacatcggcagaaactagatgatcagaccaa

SEQ ID NO: 270 - DNA, 57 bp, Artificial Sequence
  SPE Primer Pool
aatgtacagtattgcgttttgtttaggaaatccacaatactttttctgatctcttcc

SEQ ID NO: 271 - DNA, 53 bp, Artificial Sequence
  SPE Primer Pool
aatgtacagtattgcgttttggccaccaacctcattctgttttgttctctatc

SEQ ID NO: 272 - DNA, 51 bp, Artificial Sequence
  SPE Primer Pool
aatgtacagtattgcgttttgctgcatttgtcctttgactggtgtttaggt

SEQ ID NO: 273 - DNA, 52 bp, Artificial Sequence
  SPE Primer Pool
aatgtacagtattgcgttttgcttcgaccgacaaacctgaggtcattaaatc

SEQ ID NO: 274 - DNA, 46 bp, Artificial Sequence
  SPE Primer Pool
aatgtacagtattgcgttttgccccacatcccaagctaggaagacc

SEQ ID NO: 275 - DNA, 45 bp, Artificial Sequence
  SPE Primer Pool
aatgtacagtattgcgttttgcgggccagtaccttgaaagcgatg

SEQ ID NO: 276 - DNA, 51 bp, Artificial Sequence
  SPE Primer Pool
aatgtacagtattgcgttttgctaactcaatcggcttgttgtgatgcgtat

SEQ ID NO: 277 - DNA, 51 bp, Artificial Sequence
  SPE Primer Pool
aatgtacagtattgcgttttgccctcctggactgttagtaacttagtctcc

SEQ ID NO: 278 - DNA, 42 bp, Artificial Sequence
  SPE Primer Pool
aatgtacagtattgcgttttgccctccgagctccgcgaaaat

SEQ ID NO: 279 - DNA, 57 bp, Artificial Sequence
  SPE Primer Pool
aatgtacagtattgcgttttggtgctaaaaagtgtaagaagaaatgagctagcaaaa

SEQ ID NO: 280 - DNA, 55 bp, Artificial Sequence
  SPE Primer Pool
aatgtacagtattgcgttttgcatatgcctcagtttgaattcctctcacaaacaa

SEQ ID NO: 281 - DNA, 51 bp, Artificial Sequence
  SPE Primer Pool
aatgtacagtattgcgttttggggagaagaaagagagatgtagggctagag

SEQ ID NO: 282 - DNA, 54 bp, Artificial Sequence
  SPE Primer Pool
aatgtacagtattgcgttttggcaagcacttctgtttttgtcttttcagtttcg

SEQ ID NO: 283 - DNA, 57 bp, Artificial Sequence
  SPE Primer Pool
aatgtacagtattgcgttttgtctctgatatacttggattggtaattgagaaagtct

SEQ ID NO: 284 - DNA, 57 bp, Artificial Sequence
  SPE Primer Pool
aatgtacagtattgcgttttggtttgatatcttcccagcaaaataatcagctctcat

SEQ ID NO: 285 - DNA, 51 bp, Artificial Sequence
  SPE Primer Pool
aatgtacagtattgcgttttgtagccaacctcttttcgatgagctcactag

SEQ ID NO: 286 - DNA, 51 bp, Artificial Sequence
  SPE Primer Pool
aatgtacagtattgcgttttgtggaacagacaaactatcgactgaagttgt

SEQ ID NO: 287 - DNA, 48 bp, Artificial Sequence
  SPE Primer Pool
aatgtacagtattgcgttttggaggctgagtgcaaatttggtctggaa

SEQ ID NO: 288 - DNA, 50 bp, Artificial Sequence
  SPE Primer Pool
aatgtacagtattgcgttttggatggtggtggttgtctctgatgattacc

SEQ ID NO: 289 - DNA, 46 bp, Artificial Sequence
  SPE Primer Pool
aatgtacagtattgcgttttggcaaggcgagtccagaaccaagatt

SEQ ID NO: 290 - DNA, 47 bp, Artificial Sequence
  SPE Primer Pool
aatgtacagtattgcgttttgtcagaagcgactgatccccatcaagt

SEQ ID NO: 291 - DNA, 56 bp, Artificial Sequence
  SPE Primer Pool
aatgtacagtattgcgttttgcatatggtcacatcaccttaactaaacccatgttt

SEQ ID NO: 292 - DNA, 57 bp, Artificial Sequence
  SPE Primer Pool
aatgtacagtattgcgttttgtttctcggtactgtttattttgaacaaaaccaatcc

SEQ ID NO: 293 - DNA, 51 bp, Artificial Sequence
  SPE Primer Pool
aatgtacagtattgcgttttgcctcctccccaaattccaggaacaatatga

SEQ ID NO: 294 - DNA, 57 bp, Artificial Sequence
  SPE Primer Pool
aatgtacagtattgcgttttgtgtgcgtcattttatttgggaaaatttgatactaac

SEQ ID NO: 295 - DNA, 46 bp, Artificial Sequence
  SPE Primer Pool
aatgtacagtattgcgttttgcatgcaggagaagtcatcccccttc

SEQ ID NO: 296 - DNA, 51 bp, Artificial Sequence
  SPE Primer Pool
aatgtacagtattgcgttttgtctgaaaactggtggttgcctctaggttaa

SEQ ID NO: 297 - DNA, 48 bp, Artificial Sequence
  SPE Primer Pool
aatgtacagtattgcgttttggcccctttcttgctcttcttggacttg

SEQ ID NO: 298 - DNA, 48 bp, Artificial Sequence
  SPE Primer Pool
aatgtacagtattgcgttttgccaagccaagccaagctggatattgtg

SEQ ID NO: 299 - DNA, 49 bp, Artificial Sequence
  SPE Primer Pool
aatgtacagtattgcgttttgcactcacattgtgcagcttgtagtagag

SEQ ID NO: 300 - DNA, 48 bp, Artificial Sequence
  SPE Primer Pool
aatgtacagtattgcgttttggcaaagcgtctgcatttgaaggagttt

SEQ ID NO: 301 - DNA, 45 bp, Artificial Sequence
  SPE Primer Pool
aatgtacagtattgcgttttgccctcccgagaacttgccggttaa

SEQ ID NO: 302 - DNA, 46 bp, Artificial Sequence
  SPE Primer Pool
aatgtacagtattgcgttttggctccccaccacaaaaacgcaaatg

SEQ ID NO: 303 - DNA, 48 bp, Artificial Sequence
  SPE Primer Pool
aatgtacagtattgcgttttggtgtcactgacggagagcatgaagatg

SEQ ID NO: 304 - DNA, 46 bp, Artificial Sequence
  SPE Primer Pool
aatgtacagtattgcgttttgccacccaaagaagtgtctcctgacc

SEQ ID NO: 305 - DNA, 47 bp, Artificial Sequence
  SPE Primer Pool
aatgtacagtattgcgttttgtccgtcagtgacacctggtacttgac

SEQ ID NO: 306 - DNA, 48 bp, Artificial Sequence
  SPE Primer Pool
aatgtacagtattgcgttttgccctagctctgcctaccctgatctttc

SEQ ID NO: 307 - DNA, 47 bp, Artificial Sequence
  SPE Primer Pool
aatgtacagtattgcgttttgacgaggtggacgtcttcttcaatcac

SEQ ID NO: 308 - DNA, 43 bp, Artificial Sequence
  SPE Primer Pool
aatgtacagtattgcgttttggccctgcgagtcgaggtgattg

SEQ ID NO: 309 - DNA, 53 bp, Artificial Sequence
  SPE Primer Pool
aatgtacagtattgcgttttgccatgactctcaggaattggccctatacttag

SEQ ID NO: 310 - DNA, 56 bp, Artificial Sequence
  SPE Primer Pool
aatgtacagtattgcgttttgcttgggaccttcatttctatataacccctatctgg

SEQ ID NO: 311 - DNA, 49 bp, Artificial Sequence
  SPE Primer Pool
aatgtacagtattgcgttttgtgccaggaaacttttcattgtgcctctc

SEQ ID NO: 312 - DNA, 50 bp, Artificial Sequence
  SPE Primer Pool
aatgtacagtattgcgttttggttaccccatggaacttaccaagcactag

SEQ ID NO: 313 - DNA, 54 bp, Artificial Sequence
  SPE Primer Pool
aatgtacagtattgcgttttggtatgaaattcgctggagggtcattgaatcaat

SEQ ID NO: 314 - DNA, 52 bp, Artificial Sequence
  SPE Primer Pool
aatgtacagtattgcgttttgcaggaaggagcacttacgttttagcatcttc

SEQ ID NO: 315 - DNA, 57 bp, Artificial Sequence
  SPE Primer Pool
aatgtacagtattgcgttttggattttgagaaattcccttaatatccccatgctcaa

SEQ ID NO: 316 - DNA, 49 bp, Artificial Sequence
  SPE Primer Pool
aatgtacagtattgcgttttgcacaaccacatgtgtccagtgaaaatcc

SEQ ID NO: 317 - DNA, 49 bp, Artificial Sequence
  SPE Primer Pool
aatgtacagtattgcgttttgtgctttcatcagcagggttcaatccaaa

SEQ ID NO: 318 - DNA, 57 bp, Artificial Sequence
  SPE Primer Pool
aatgtacagtattgcgttttgcatttacatcatcacagagtattgcttctatggaga

SEQ ID NO: 319 - DNA, 56 bp, Artificial Sequence
  SPE Primer Pool
aatgtacagtattgcgttttggtgatctctggatgtcggaatatttagaaacctct

SEQ ID NO: 320 - DNA, 57 bp, Artificial Sequence
  SPE Primer Pool
aatgtacagtattgcgttttgatcttttgaaaacaatggtgactacatggacatgaa

SEQ ID NO: 321 - DNA, 54 bp, Artificial Sequence
  SPE Primer Pool
aatgtacagtattgcgttttgggtctaaaaaggtctgtgttccttgaacttaca

SEQ ID NO: 322 - DNA, 56 bp, Artificial Sequence
  SPE Primer Pool
aatgtacagtattgcgttttgccagcaccaatacatttaatttcttttctgcagac

SEQ ID NO: 323 - DNA, 51 bp, Artificial Sequence
  SPE Primer Pool
aatgtacagtattgcgttttggctacagatggcttgatcctgagtcatttc

SEQ ID NO: 324 - DNA, 48 bp, Artificial Sequence
  SPE Primer Pool
aatgtacagtattgcgttttggtcaggcccataccaagggaaaagatc

SEQ ID NO: 325 - DNA, 51 bp, Artificial Sequence
  SPE Primer Pool
aatgtacagtattgcgttttgacactgagtgatgtctggtcttatggcatt

SEQ ID NO: 326 - DNA, 50 bp, Artificial Sequence
  SPE Primer Pool
aatgtacagtattgcgttttgcactgagcgtttgttagtcctggtgtttt

SEQ ID NO: 327 - DNA, 53 bp, Artificial Sequence
  SPE Primer Pool
aatgtacagtattgcgttttgcagattctccacaatctcactcaggtggtaaa

SEQ ID NO: 328 - DNA, 49 bp, Artificial Sequence
  SPE Primer Pool
aatgtacagtattgcgttttgccccacagctacgagatcatggtgaaat

SEQ ID NO: 329 - DNA, 57 bp, Artificial Sequence
  SPE Primer Pool
aatgtacagtattgcgttttgtctctattcatttttgaggtttggttgttaacactt

SEQ ID NO: 330 - DNA, 48 bp, Artificial Sequence
  SPE Primer Pool
aatgtacagtattgcgttttggggagtgcaccattatcgggaaaatgg

SEQ ID NO: 331 - DNA, 56 bp, Artificial Sequence
  SPE Primer Pool
aatgtacagtattgcgttttggcttattctcattcgtttcatccaggatctcaaaa

SEQ ID NO: 332 - DNA, 46 bp, Artificial Sequence
  SPE Primer Pool
aatgtacagtattgcgttttggggcgacgagattaggctgttatgc

SEQ ID NO: 333 - DNA, 49 bp, Artificial Sequence
  SPE Primer Pool
aatgtacagtattgcgttttgcccctctgcattataagcagtgccaaaa

SEQ ID NO: 334 - DNA, 50 bp, Artificial Sequence
  SPE Primer Pool
aatgtacagtattgcgttttggcccacatcgttgtaagccttacattcaa

SEQ ID NO: 335 - DNA, 50 bp, Artificial Sequence
  SPE Primer Pool
aatgtacagtattgcgttttgccgtttggaaagctagtggttcagagttc

SEQ ID NO: 336 - DNA, 50 bp, Artificial Sequence
  SPE Primer Pool
aatgtacagtattgcgttttggagatcccatcctgccaaagtttgtgatt

SEQ ID NO: 337 - DNA, 50 bp, Artificial Sequence
  SPE Primer Pool
aatgtacagtattgcgttttgggaaagcccctgtttcatactgaccaaaa

SEQ ID NO: 338 - DNA, 51 bp, Artificial Sequence
  SPE Primer Pool
aatgtacagtattgcgttttgctttctccccacagaaacccatgtatgaag

SEQ ID NO: 339 - DNA, 51 bp, Artificial Sequence
  SPE Primer Pool
aatgtacagtattgcgttttggtttgccagttgtgctttttgctaaaatgc

SEQ ID NO: 340 - DNA, 48 bp, Artificial Sequence
  SPE Primer Pool
aatgtacagtattgcgttttgccctcccaccctcaggactataccaat

SEQ ID NO: 341 - DNA, 46 bp, Artificial Sequence
  SPE Primer Pool
aatgtacagtattgcgttttgtgctcggcagattggtatagtcctg

SEQ ID NO: 342 - DNA, 50 bp, Artificial Sequence
  SPE Primer Pool
aatgtacagtattgcgttttgggcatcctctgtcctatctcccagataca

SEQ ID NO: 343 - DNA, 56 bp, Artificial Sequence
  SPE Primer Pool
aatgtacagtattgcgttttgaggttttatactaaacttactttgactgggtttgg

SEQ ID NO: 344 - DNA, 45 bp, Artificial Sequence
  SPE Primer Pool
aatgtacagtattgcgttttgcccccagaggtaagcgtcatatgg

SEQ ID NO: 345 - DNA, 49 bp, Artificial Sequence
  SPE Primer Pool
aatgtacagtattgcgttttggcacagggaagtaggtactgggagattg

SEQ ID NO: 346 - DNA, 48 bp, Artificial Sequence
  SPE Primer Pool
aatgtacagtattgcgttttgaggcctgcaaggttttaactggaccta

SEQ ID NO: 347 - DNA, 49 bp, Artificial Sequence
  SPE Primer Pool
aatgtacagtattgcgttttgcgggagctgataagtggtacctgtatgt

SEQ ID NO: 348 - DNA, 49 bp, Artificial Sequence
  SPE Primer Pool
aatgtacagtattgcgttttggaaaagggtcccaggtaggtccagttaa

SEQ ID NO: 349 - DNA, 57 bp, Artificial Sequence
  SPE Primer Pool
aatgtacagtattgcgttttgctctcggtgtatttctctacttacctgtaataatgc

SEQ ID NO: 350 - DNA, 57 bp, Artificial Sequence
  SPE Primer Pool
aatgtacagtattgcgttttgtttattgatgtctatgaagtgttgtggttccttaac

SEQ ID NO: 351 - DNA, 49 bp, Artificial Sequence
  SPE Primer Pool
aatgtacagtattgcgttttgcagaaaacaagctgccgcaaagttctac

SEQ ID NO: 352 - DNA, 48 bp, Artificial Sequence
  SPE Primer Pool
aatgtacagtattgcgttttgcaggtgttgcgatgatgtcactgtacg

SEQ ID NO: 353 - DNA, 57 bp, Artificial Sequence
  SPE Primer Pool
aatgtacagtattgcgttttgtcatttttcattggacttgttttgtcagctttttgg

SEQ ID NO: 354 - DNA, 55 bp, Artificial Sequence
  SPE Primer Pool
aatgtacagtattgcgttttggttagccccaatatgaaaaataaagctggttgga

SEQ ID NO: 355 - DNA, 53 bp, Artificial Sequence
  SPE Primer Pool
aatgtacagtattgcgttttgctggttggaggtttttgctaaatctggaatga

SEQ ID NO: 356 - DNA, 57 bp, Artificial Sequence
  SPE Primer Pool
aatgtacagtattgcgttttgttctttttgactagaaaacttcagccactgtgtatt

SEQ ID NO: 357 - DNA, 55 bp, Artificial Sequence
  SPE Primer Pool
aatgtacagtattgcgttttgcatatgaccaattgcagatgagcccattattgaa

SEQ ID NO: 358 - DNA, 51 bp, Artificial Sequence
  SPE Primer Pool
aatgtacagtattgcgttttgaggcatagctgactcatctatgtttgttct

SEQ ID NO: 359 - DNA, 57 bp, Artificial Sequence
  SPE Primer Pool
aatgtacagtattgcgttttgttcctcatttctttcactctgacagtataaaggtaa

SEQ ID NO: 360 - DNA, 54 bp, Artificial Sequence
  SPE Primer Pool
aatgtacagtattgcgttttggaactattccaacagaacaaaccgataacatca

SEQ ID NO: 361 - DNA, 53 bp, Artificial Sequence
  SPE Primer Pool
aatgtacagtattgcgttttgtggatagcaagacaattagagcccaacttagt

SEQ ID NO: 362 - DNA, 54 bp, Artificial Sequence
  SPE Primer Pool
aatgtacagtattgcgttttgctactcctcctgtctctttccacatcatcaatt

SEQ ID NO: 363 - DNA, 57 bp, Artificial Sequence
  SPE Primer Pool
aatgtacagtattgcgttttgaggaccttatgttgtatgctgtataaatctaaaggt

SEQ ID NO: 364 - DNA, 57 bp, Artificial Sequence
  SPE Primer Pool
aatgtacagtattgcgttttggtttgtcatcttctatggtaagtatctttctggatg

SEQ ID NO: 365 - DNA, 57 bp, Artificial Sequence
  SPE Primer Pool
aatgtacagtattgcgttttgtggaggagaaacagataaaagttgagtatacgttta

SEQ ID NO: 366 - DNA, 54 bp, Artificial Sequence
  SPE Primer Pool
aatgtacagtattgcgttttggaggatgacgacatgttagtaagcactactact

SEQ ID NO: 367 - DNA, 57 bp, Artificial Sequence
  SPE Primer Pool
aatgtacagtattgcgttttgattccaccatcatttccttctccaaaattatcatcc

SEQ ID NO: 368 - DNA, 53 bp, Artificial Sequence
  SPE Primer Pool
aatgtacagtattgcgttttgctcaaaagcactgccttctctcattatctcac

SEQ ID NO: 369 - DNA, 57 bp, Artificial Sequence
  SPE Primer Pool
aatgtacagtattgcgttttgaatgtatttgaccttcttttaaagtgacatcgatgt

SEQ ID NO: 370 - DNA, 55 bp, Artificial Sequence
  SPE Primer Pool
aatgtacagtattgcgttttgtgatgttcccaacttcttctctcatggttatctc

SEQ ID NO: 371 - DNA, 56 bp, Artificial Sequence
  SPE Primer Pool
aatgtacagtattgcgttttgccctctgatccctagataatttatgggtagctaga

SEQ ID NO: 372 - DNA, 55 bp, Artificial Sequence
  SPE Primer Pool
aatgtacagtattgcgttttgcacgaaatgcaggttttggaatatgattaatgtt

SEQ ID NO: 373 - DNA, 56 bp, Artificial Sequence
  SPE Primer Pool
aatgtacagtattgcgttttggaacaatgttctacgcacattttgttctcagtaaa

SEQ ID NO: 374 - DNA, 50 bp, Artificial Sequence
  SPE Primer Pool
aatgtacagtattgcgttttgtccacgctgctctctaaattacactcgaa

SEQ ID NO: 375 - DNA, 54 bp, Artificial Sequence
  SPE Primer Pool
aatgtacagtattgcgttttgacgtagaacacatttcattttactcctctttgg

SEQ ID NO: 376 - DNA, 57 bp, Artificial Sequence
  SPE Primer Pool
aatgtacagtattgcgttttggtcacatgaatgtaaatcaagaaaacagatgttgtt

SEQ ID NO: 377 - DNA, 57 bp, Artificial Sequence
  SPE Primer Pool
aatgtacagtattgcgttttgttctgaactatttatggacaacagtcaaacaacaat

SEQ ID NO: 378 - DNA, 57 bp, Artificial Sequence
  SPE Primer Pool
aatgtacagtattgcgttttgtgaagccattgcgagaactttatccataagtatttc

SEQ ID NO: 379 - DNA, 51 bp, Artificial Sequence
  SPE Primer Pool
aatgtacagtattgcgttttggccagagcacatgaataaatgagcatccat

SEQ ID NO: 380 - DNA, 53 bp, Artificial Sequence
  SPE Primer Pool
aatgtacagtattgcgttttgggaagctctcagggtacaaattctcagatcat

SEQ ID NO: 381 - DNA, 54 bp, Artificial Sequence
  SPE Primer Pool
aatgtacagtattgcgttttgctcagggtacaaattctcagatcatcagtcctc

SEQ ID NO: 382 - DNA, 49 bp, Artificial Sequence
  SPE Primer Pool
aatgtacagtattgcgttttgctctacacaagcttcctttccgtcatgc

SEQ ID NO: 383 - DNA, 52 bp, Artificial Sequence
  SPE Primer Pool
aatgtacagtattgcgttttgcccttcagatcttctcagcattcgagagatc

SEQ ID NO: 384 - DNA, 49 bp, Artificial Sequence
  SPE Primer Pool
aatgtacagtattgcgttttgaatcgaagcgctacctgattccaattcc

SEQ ID NO: 385 - DNA, 46 bp, Artificial Sequence
  SPE Primer Pool
aatgtacagtattgcgttttgccgaccgtaactattcggtgcgttg

SEQ ID NO: 386 - DNA, 57 bp, Artificial Sequence
  SPE Primer Pool
aatgtacagtattgcgttttgacattctatccaagctgtgttctatcttgagaaact

SEQ ID NO: 387 - DNA, 48 bp, Artificial Sequence
  SPE Primer Pool
aatgtacagtattgcgttttgcgagtgagggttttcgtggttcacatc

SEQ ID NO: 388 - DNA, 45 bp, Artificial Sequence
  SPE Primer Pool
aatgtacagtattgcgttttgcgtgggtcccagtctgcagttaag

SEQ ID NO: 389 - DNA, 44 bp, Artificial Sequence
  SPE Primer Pool
aatgtacagtattgcgttttggctcagagccgttccgagatctt

SEQ ID NO: 390 - DNA, 48 bp, Artificial Sequence
  SPE Primer Pool
aatgtacagtattgcgttttggcgttccatctcccacttgtcgtagtt

SEQ ID NO: 391 - DNA, 49 bp, Artificial Sequence
  SPE Primer Pool
aatgtacagtattgcgttttgctggccgagttggttcatcatcattcaa

SEQ ID NO: 392 - DNA, 48 bp, Artificial Sequence
  SPE Primer Pool
aatgtacagtattgcgttttgtatggtgtgtcccccaactacgacaag

SEQ ID NO: 393 - DNA, 57 bp, Artificial Sequence
  SPE Primer Pool
aatgtacagtattgcgttttgtgaaaagcacttcctgaaataatttcaccttcgttt

SEQ ID NO: 394 - DNA, 47 bp, Artificial Sequence
  SPE Primer Pool
aatgtacagtattgcgttttgaggtactccatggctgacgagatctg

SEQ ID NO: 395 - DNA, 48 bp, Artificial Sequence
  SPE Primer Pool
aatgtacagtattgcgttttgttgcctttgttccaaggtccaatgtgt

SEQ ID NO: 396 - DNA, 43 bp, Artificial Sequence
  SPE Primer Pool
aatgtacagtattgcgttttgcgtccccgcattccaacgtctc

SEQ ID NO: 397 - DNA, 42 bp, Artificial Sequence
  SPE Primer Pool
aatgtacagtattgcgttttgggcgcgccgtttacttgaagg

SEQ ID NO: 398 - DNA, 44 bp, Artificial Sequence
  SPE Primer Pool
aatgtacagtattgcgttttggcctggcggtgcacactattctg

SEQ ID NO: 399 - DNA, 48 bp, Artificial Sequence
  SPE Primer Pool
aatgtacagtattgcgttttgaggtgcagccacaaaacttacagatgc

SEQ ID NO: 400 - DNA, 45 bp, Artificial Sequence
  SPE Primer Pool
aatgtacagtattgcgttttggtgccgaaccaatacaaccctctg

SEQ ID NO: 401 - DNA, 43 bp, Artificial Sequence
  SPE Primer Pool
aatgtacagtattgcgttttggggcgggtccaccagtttgaat

SEQ ID NO: 402 - DNA, 44 bp, Artificial Sequence
  SPE Primer Pool
aatgtacagtattgcgttttgccgcagagggttgtattggttcg

SEQ ID NO: 403 - DNA, 47 bp, Artificial Sequence
  SPE Primer Pool
aatgtacagtattgcgttttgagccactcgcattgaccattcaaact

SEQ ID NO: 404 - DNA, 44 bp, Artificial Sequence
  SPE Primer Pool
aatgtacagtattgcgttttgccacgtctgacaggtagccatgg

SEQ ID NO: 405 - DNA, 45 bp, Artificial Sequence
  SPE Primer Pool
aatgtacagtattgcgttttggtgaggctgctggacgagtacaac

SEQ ID NO: 406 - DNA, 43 bp, Artificial Sequence
  SPE Primer Pool
aatgtacagtattgcgttttgcgcaccaggttgtactcgtcca

SEQ ID NO: 407 - DNA, 46 bp, Artificial Sequence
  SPE Primer Pool
aatgtacagtattgcgttttgccgcctttgtgcttctgttcttcgt

SEQ ID NO: 408 - DNA, 57 bp, Artificial Sequence
  SPE Primer Pool
aatgtacagtattgcgttttgctgattaatcgcgtagaaaatgaccttattttggag

SEQ ID NO: 409 - DNA, 49 bp, Artificial Sequence
  SPE Primer Pool
aatgtacagtattgcgttttggctccatcgtctacctggagattgacaa

SEQ ID NO: 410 - DNA, 45 bp, Artificial Sequence
  SPE Primer Pool
aatgtacagtattgcgttttgtctgcacggcctcgatcttgtagg

SEQ ID NO: 411 - DNA, 48 bp, Artificial Sequence
  SPE Primer Pool
aatgtacagtattgcgttttggccagcagatgatcttcccctactacg

SEQ ID NO: 412 - DNA, 44 bp, Artificial Sequence
  SPE Primer Pool
aatgtacagtattgcgttttgcgtcacgcttgaagaccacgttg

SEQ ID NO: 413 - DNA, 45 bp, Artificial Sequence
  SPE Primer Pool
aatgtacagtattgcgttttggccagcatgcagttctaaggctct

SEQ ID NO: 414 - DNA, 43 bp, Artificial Sequence
  SPE Primer Pool
aatgtacagtattgcgttttggtgcccgtctcgactcttaggc

SEQ ID NO: 415 - DNA, 49 bp, Artificial Sequence
  SPE Primer Pool
aatgtacagtattgcgttttgtgtagccgctgatcgtcgtgtatatgtc

SEQ ID NO: 416 - DNA, 57 bp, Artificial Sequence
  SPE Primer Pool
aatgtacagtattgcgttttggactggtactggttagtaaaggttgataatattcca

SEQ ID NO: 417 - DNA, 57 bp, Artificial Sequence
  SPE Primer Pool
aatgtacagtattgcgttttgggtgaagtaatcagtttgttcactagttacgtgatt

SEQ ID NO: 418 - DNA, 57 bp, Artificial Sequence
  SPE Primer Pool
aatgtacagtattgcgttttgctgacatgcctactgattattcttcaaactcatcac

SEQ ID NO: 419 - DNA, 57 bp, Artificial Sequence
  SPE Primer Pool
aatgtacagtattgcgttttgtgtgtgttttaattgttccacttgagattcttaacc

SEQ ID NO: 420 - DNA, 57 bp, Artificial Sequence
  SPE Primer Pool
aatgtacagtattgcgttttgcgtcagcattttgaatcacttcattctgacatgata

SEQ ID NO: 421 - DNA, 57 bp, Artificial Sequence
  SPE Primer Pool
aatgtacagtattgcgttttgagtaattttcaactattggcctagtgaatttaagct

SEQ ID NO: 422 - DNA, 55 bp, Artificial Sequence
  SPE Primer Pool
aatgtacagtattgcgttttgagaaagagggaagtcacatttatagagtgctagc

SEQ ID NO: 423 - DNA, 57 bp, Artificial Sequence
  SPE Primer Pool
aatgtacagtattgcgttttgcatcaacagaaacagaacaacaaactgtgacaaatc

SEQ ID NO: 424 - DNA, 57 bp, Artificial Sequence
  SPE Primer Pool
aatgtacagtattgcgttttgccaaagaatatccctttatatagcagtggaacaatt

SEQ ID NO: 425 - DNA, 55 bp, Artificial Sequence
  SPE Primer Pool
aatgtacagtattgcgttttgcagaatatgcagtgataagtgctgtttcatcact

SEQ ID NO: 426 - DNA, 48 bp, Artificial Sequence
  SPE Primer Pool
aatgtacagtattgcgttttgttccccctgtgacgactacttttcctc

SEQ ID NO: 427 - DNA, 48 bp, Artificial Sequence
  SPE Primer Pool
aatgtacagtattgcgttttgcggtccctatttcttcctctgcttcgt

SEQ ID NO: 428 - DNA, 52 bp, Artificial Sequence
  SPE Primer Pool
aatgtacagtattgcgttttgctgaacagttctgtctctattacccgacctc

SEQ ID NO: 429 - DNA, 52 bp, Artificial Sequence
  SPE Primer Pool
aatgtacagtattgcgttttgcgttcatagccttctatccgagtatgtagca

SEQ ID NO: 430 - DNA, 47 bp, Artificial Sequence
  SPE Primer Pool
aatgtacagtattgcgttttgccccttctgtcctcgcaggttaatcc

SEQ ID NO: 431 - DNA, 53 bp, Artificial Sequence
  SPE Primer Pool
aatgtacagtattgcgttttggcttccagccatttctgagatatcctcacagt

SEQ ID NO: 432 - DNA, 52 bp, Artificial Sequence
  SPE Primer Pool
aatgtacagtattgcgttttgaccaggaggaacaaagacacatgaagatcat

SEQ ID NO: 433 - DNA, 45 bp, Artificial Sequence
  SPE Primer Pool
aatgtacagtattgcgttttggcgcccccgagtttcttacgaatc

SEQ ID NO: 434 - DNA, 57 bp, Artificial Sequence
  SPE Primer Pool
aatgtacagtattgcgttttgtttatacacagtttggagtttgagaatcagaagact

SEQ ID NO: 435 - DNA, 57 bp, Artificial Sequence
  SPE Primer Pool
aatgtacagtattgcgttttgggttatctctggctgatgagattatgagtgattctc

SEQ ID NO: 436 - DNA, 51 bp, Artificial Sequence
  SPE Primer Pool
aatgtacagtattgcgttttggccaagctagtgattgatgtgattcgctat

SEQ ID NO: 437 - DNA, 47 bp, Artificial Sequence
  SPE Primer Pool
aatgtacagtattgcgttttgcccctcctctagtactccctgtttgt

SEQ ID NO: 438 - DNA, 51 bp, Artificial Sequence
  SPE Primer Pool
aatgtacagtattgcgttttgctccttcctgtcccaatcaactagtctagc

SEQ ID NO: 439 - DNA, 46 bp, Artificial Sequence
  SPE Primer Pool
aatgtacagtattgcgttttggcctcgtccctcttcccttaggtaa

SEQ ID NO: 440 - DNA, 55 bp, Artificial Sequence
  SPE Primer Pool
aatgtacagtattgcgttttgtctctcttcccattagtctgagtactgagtgatt

SEQ ID NO: 441 - DNA, 56 bp, Artificial Sequence
  SPE Primer Pool
aatgtacagtattgcgttttgagcatttcttgagacttaaagtggcattctaaagg

SEQ ID NO: 442 - DNA, 57 bp, Artificial Sequence
  SPE Primer Pool
aatgtacagtattgcgttttgatttttattctcaagaggcagaaataccaacttacc

SEQ ID NO: 443 - DNA, 57 bp, Artificial Sequence
  SPE Primer Pool
aatgtacagtattgcgttttgaatttatagctcttttcatctgctttggtatcatca

SEQ ID NO: 444 - DNA, 56 bp, Artificial Sequence
  SPE Primer Pool
aatgtacagtattgcgttttggcctctaatctgatatacagccttagaaagtcaca

SEQ ID NO: 445 - DNA, 47 bp, Artificial Sequence
  SPE Primer Pool
aatgtacagtattgcgttttgtgtgccattgtcctggagcaacaatt

SEQ ID NO: 446 - DNA, 57 bp, Artificial Sequence
  SPE Primer Pool
aatgtacagtattgcgttttgagtgtactgctcgttttcttaatttgaaaagtgagt

SEQ ID NO: 447 - DNA, 57 bp, Artificial Sequence
  SPE Primer Pool
aatgtacagtattgcgttttgacccatgaactaatacttattttgagattggtccat

SEQ ID NO: 448 - DNA, 55 bp, Artificial Sequence
  SPE Primer Pool
aatgtacagtattgcgttttgcatggtgcaacaaaagtaagaatccaacagtttt

SEQ ID NO: 449 - DNA, 57 bp, Artificial Sequence
  SPE Primer Pool
aatgtacagtattgcgttttgttgaaatgttaagtaagcttgaaataccgatagcat

SEQ ID NO: 450 - DNA, 51 bp, Artificial Sequence
  SPE Primer Pool
aatgtacagtattgcgttttggggaggaagaaaatgaagcacgaggaaaac

SEQ ID NO: 451 - DNA, 57 bp, Artificial Sequence
  SPE Primer Pool
aatgtacagtattgcgttttgatttgggatgtactctaaatttaaagcagcaaatca

SEQ ID NO: 452 - DNA, 57 bp, Artificial Sequence
  SPE Primer Pool
aatgtacagtattgcgttttgtcaagagcagaatttggagactttgatattaaaact

SEQ ID NO: 453 - DNA, 57 bp, Artificial Sequence
  SPE Primer Pool
aatgtacagtattgcgttttgcggttactaacatgtttagggaaatagacaactgtt

SEQ ID NO: 454 - DNA, 57 bp, Artificial Sequence
  SPE Primer Pool
aatgtacagtattgcgttttgcctgacaacagatcccatataattaactttcatacc

SEQ ID NO: 455 - DNA, 55 bp, Artificial Sequence
  SPE Primer Pool
aatgtacagtattgcgttttgagatgaagaagatgaggaacgagagagtaaaagc

(19) United States
(12) Patent Application Publication
(10) Pub. No.: US 2022/0127600 A1
(43) Pub. Date: Apr. 28, 2022

(54) Methods of Detecting Analytes and Compositions Thereof

(71) Applicant: QIAGEN Sciences, LLC, Germantown, MD (US)

(72) Inventors: Yexun WANG, Ellicott City, MD (US); Quan PENG, Clarksburg, MD (US)

(21) Appl. No.: 17/421,617

(22) PCT Filed: Jan. 9, 2020
(86) PCT No.: PCT/US2020/012892

§ 371 Date: Jul. 8, 2021

Related U.S. Application Data
(60) Provisional application No. 62/790,338, filed on Jan. 9, 2019.

(51) Int. Cl. C12N 15/10

(52) U.S. Cl. CPC C12N 15/1093; C12Q 1/6806

(57) ABSTRACT

The invention relates to methods of detecting analytes in samples by generating analyte-based DNA libraries amenable for sequencing. The methods include the use of proximity probe pairs, each probe comprising an analyte binding domain and oligonucleotide domain. The methods further provide for integrated DNA and RNA library preparations and methods of making and uses thereof. The invention also provides compositions useful in the methods.

### BACKGROUND OF THE INVENTION

Next-generation sequencing (NGS) technology has been used for nucleic acid analysis, e.g., in DNA variant detection as well as in RNA transcriptome profiling. Equally important to DNA/RNA are protein biomarkers in translational research. However, most protein analysis are done on completely different platforms. For example, protein analysis can be done through traditional ELISA assay or mass spectrometry assays. Being able to analyze nucleic acid and protein biomarkers on the same platform would significantly reduce the analysis time and provide more insights.

People have successfully converted protein detection into nucleic acid detection through the use of oligonucleotide conjugated antibodies (Ab). Immuno-PCR is one such technology described decades ago (Sano, T. et al., Science 258:120-2 (1992)). In this case, the antigen specific Ab is conjugated to a oligonucleotide sequence and is used in a typical ELISA process. Although there are many variations of ELISA, the process typically involve, at a minimum, antigen antibody binding, antibody washing and detection steps. In the case of Immuno-PCR, the final detection is done by using a real-time PCR assay to quantify specific oligonucleotides conjugated to antibodies bound to specific antigen. Comparing to ELISA with traditional colorimetric readout, Immuno-PCR is theoretically more sensitive because real-time PCR can detect even a minute amount of oligonucleotides specifically bound to antigen. Immuno-PCR also has higher multiplexing potentials, because different oligonucleotide sequences can be used to detect different antigen-antibody pairs. However in practice, due to non-specific binding of antibodies, the real Immuno-PCR sensitivity is usually limited to antibody specificity. Furthermore, due to the inherent variability from exponential amplification, real-time PCR is not very accurate for detecting small changes in abundance, e.g., there is high variability in measuring 50% change or less than 1 Ct difference in real-time PCR.

To address limitations in Immuno-PCR, people has developed protein proximity ligation (PLA) and proximity extension (PEA) assays (Gullberg, M. et al., Proc. Natl. Acad. Sci. USA. 101:8420-4 (2004); Lundberg, M. et al., Nucleic Acids Res. 39: e102 (2011)). In both technologies, a pair of antibodies to the same antigen is conjugated with different oligonucleotides. When the pair of antibodies bind to specific antigens, the conjugated oligonucleotides are brought to close proximity, much closer than they would be randomly in solution. Due to this close proximity, these two oligonucleotides now have a higher likelihood to engage in intermolecular ligation or extension reaction. The resulting ligation or extension products can be detected using, e.g., PCR assays. Because the proximity is controlled by the specificity of two antibodies, proximity assays can be more specific and often do not require extensive wash step to remove unbound antibodies. However, existing PLA and PEA assays are still affected by the same limitations of the downstream qPCR detection, being not very reliable in detecting small differences.

Using NGS as a downstream readout for PLA assays is known (Darmanis S. et al., PLoS One. 6:e25583 (2011). This method could potentially increase assay throughput, so that large numbers of protein targets across many samples can be analyzed on a single platform. However, the read counting can still be inaccurate due to extensive amplification bias in the NGS sample preparation workflow.

There remains a need for improved, protein analysis methods, amenable for sequencing analysis.

### BRIEF SUMMARY OF THE INVENTION

Disclosed herein are methods for detecting an analyte in a sample, comprising: attaching first and second proximity probes to an analyte in the sample, wherein the first proximity probe comprises a first analyte binding domain and a first oligonucleotide domain comprising a universal amplification region, a variable probe specific tag region (PST), a unique molecular identifier (UMI), and an inter-molecular reacting region (IMR), and wherein the second proximity probe comprises a second analyte binding domain and a second oligonucleotide domain comprises a universal amplification region, a PST, and an IMR; and detecting the analyte. In some embodiments, the oligonucleotide domain of the second proximity probe further comprises a UMI.

The first and second analyte binding domains can be but are not limited to antibodies, aptamers, ligands, receptors, or a combination therof. The first and second analyte binding domains can be conjugated to the oligonucleotide domains, e.g., by a chemical bond, hybridization to an intermediary oligonucleotide linked to the analyte binding domain, streptavidin, biotin, or a combination thereof. In some embodiments, the first and second analyte binding domains are first and second antibodies, respectively. Each of the first and second antibodies can be one polyclonal antibody divided into two antibodies, two different polyclonal antibodies, two different monoclonal antibodies, or a combination thereof.

The methods can further comprise performing a proximity ligation (PLA) or extension (PEA) assay. The PLA or PEA assay can generate a third oligonucleotide that is single-stranded or double-stranded.

The methods can further comprise attaching an adapter sequence to the third oligonucleotide. The adapter sequence can be attached to the third oligonucleotide by amplification or ligation.

The methods can further comprise performing amplification of the third oligonucleotide to generate a protein-based DNA library.

The methods can further comprise preparing DNA and cDNA libraries from the same sample, comprising: ligating a DNA tag to an end of a DNA molecule in the sample, wherein the DNA tag comprises a UMI and a DNA identifier; and performing reverse transcription of a RNA molecule in the sample in the presence of a RNA tag, wherein the RNA tag comprises a RNA identifier, a UMI, and a poly(T). The reverse transcription can be performed in the presence of a second RNA tag, wherein the second RNA tag comprises a RNA identifier, a UMI, and a template switching oligonucleotide (TSO).

The methods can further comprise amplifying the tagged DNA and the tagged cDNA for enrichment with a set of gene specific primers. The methods can further comprise separating the amplified sample into first, second, or third sample. The protein, DNA and RNA molecules can be obtained from a biological sample, e.g., the same biological sample. In some embodiments, the DNA and RNA molecules are fragmented DNA and RNA from the biological sample. In some embodiments, the DNA molecule contains polished ends for ligation. In other embodiments, the RNA molecule is polyadenylated.

In some embodiments, the method does not require ribosomal depletion.

The methods can further comprise amplifying the first sample with primers specific for the DNA tag. The amplification can generate a DNA library corresponding to the DNA in the sample.

The methods can further comprise amplifying the second sample with primers specific for the RNA tag. The amplification can generate a cDNA library corresponding to the RNA in a sample.

The methods can further comprise sequencing the protein-based DNA, DNA, or cDNA library. The DNA molecule can be genomic DNA. The DNA library can be used for DNA variant detection, copy number analysis, fusion gene detection, or structural variant detection. The cDNA library can be used for RNA variant detection, gene expression analysis, or fusion gene detection. The DNA and cDNA libraries can be used for paired DNA and RNA profiling.

In some embodiments, the third oligonucleotide is separated from the genomic DNA and total RNA.

The methods can further comprise: (a) obtaining purified DNA and RNA from the same biological sample; (b) attaching a DNA tag sequence to the DNA in the sample; (c) attaching an RNA tag sequence to the RNA in the sample; and (d) detecting DNA, RNA and protein targets, respectively.

Also disclosed herein are protein-based DNA libraries made by any of the methods disclosed herein. Further disclosed are DNA libraries made by any of the method disclosed herein. Further disclosed are cDNA libraries made by any of the methods disclosed herein.

Disclosed herein are compositions comprising a first proximity probe comprising a first analyte binding domain and a first oligonucleotide domain comprising a universal amplification region, a variable probe specific tag region (PST), a unique molecular identifier (UMI), and an inter-molecular reacting region (IMR), and a second proximity probe comprising a second analyte binding domain and a second oligonucleotide domain comprises a universal amplification region, a PST, and an IMR. The second oligonucleotide domain can further comprise a unique molecular identifier (UMI). The first and second analyte binding domains can be antibodies, aptamers, ligands, receptors, or a combination thereof. The first and second analyte binding domains can be conjugate to the oligonucleotide domains by a chemical bond, hybridization to an intermediary oligonucleotide linked to the analyte binding domain, streptavidin, biotin, or a combination thereof. The first and second analyte binding domains can be first and second antibodies, respectively. Each of the first and second antibodies can be one polyclonal antibody divided into two antibodies, two different polyclonal antibodies, two different monoclonal antibodies, or a combination thereof.

The compositions can further comprise a DNA tag comprising a unique molecular identifier (UMI) and a DNA identifier, and/or a RNA tag comprising a RNA identifier, a UMI, and a poly(T). The compositions can further comprise a RNA tag comprising a RNA identifier, a UMI, and a template switching oligonucleotide (TSO). The DNA tag can comprise the UMI and the DNA identifier in a 5′ to 3′ direction. The RNA tag can comprise the RNA identifier, the UMI, and the poly(T) in a 5′ to 3′ direction. The RNA tag can comprise the RNA identifier, the UMI, and the TSO in a 5′ to 3′ direction.

### BRIEF DESCRIPTION OF THE DRAWINGS/FIGURES

FIG. 1. Exemplary pair of proximity probes.

FIG. 2. Workflow showing PEA using one probe bearing a UMI. The free 3′ end is shown with arrow.

FIG. 3. Third oligonucleotide generated from a proximity reaction.

FIG. 4. Flowchart of proximity assay.

FIG. 5. Exemplary DNA and RNA tag molecules.

FIG. 6. Exemplary process for generating DNA and cDNA libraries.

### DETAILED DESCRIPTION OF THE INVENTION

Disclosed herein are improved PLA and PEA assay designs to incorporate unique molecular index (UMI) and protein or analyte specific tag sequences. NGS can be used to count UMI as a way of counting protein abundance. Protein or analyte PLA or PEA assays with UMI can be performed with genomic DNA/transcriptome RNA library preparation from the same sample input, i.e., DNA/RNA/protein biomarkers can be quantitatively analyzed on the same NGS platform by counting respective UMIs. Combined workflows for simultaneous DNA and RNA enrichment and library preparation without requiring physical separation of genomic DNA and total RNA are reported in U.S. Appl. No. 62/648,174, filed Mar. 26, 2018, the entirety of which is incorporated herein by reference. The new UMI enabled PLA and PEA assay designs can be incorporated therein to allow the analysis of protein/DNA/RNA simultaneously, all from the same sample.

Disclosed herein are methods for detecting an analyte in a sample, comprising: attaching first and second proximity probes to an analyte in the sample, wherein the first proximity probe comprises a first analyte binding domain and a first oligonucleotide domain comprising a universal amplification region, a variable probe specific tag region (PST), a unique molecular identifier (UMI), and an inter-molecular reacting region (IMR), and wherein the second proximity probe comprises a second analyte binding domain and a second oligonucleotide domain comprises a universal amplification region, a PST, and an IMR; and detecting the analyte. The methods can further comprise performing a proximity ligation (PLA) or extension (PEA) assay. Methods for performing PLA and PEA are well known in the art.

The PLA or PEA assay generates a third oligonucleotide that is single-stranded or double-stranded. The methods can further comprise performing amplification of the third oligonucleotide to generate a protein-based DNA library.

Also disclosed herein are compositions comprising a first proximity probe comprising a first analyte binding domain and a first oligonucleotide domain comprising a universal amplification region, a variable probe specific tag region (PST), a unique molecular identifier (UMI), and an inter-molecular reacting region (IMR), and a second proximity probe comprising a second analyte binding domain and a second oligonucleotide domain comprises a universal amplification region, a PST, and an IMR.

In some embodiments, the second oligonucleotide domain of the second proximity probe further comprises a UMI.

In the first and second proximity probes, the first and second analyte binding domains, respectively, can be antibodies, aptamers, ligands, receptors, or a combination thereof.

In some embodiments, the first and second analyte binding domains are conjugate to the first and second oligonucleotide domains, respectively, by a chemical bond, hybridization to an intermediary oligonucleotide linked to the analyte binding domain, streptavidin, biotin, or a combination thereof.

In some embodiments, the first and second analyte binding domains can be first and second antibodies, respectively. For example, each of the first and second antibodies is one polyclonal antibody divided into two antibodies, two different polyclonal antibodies, two different monoclonal antibodies, or a combination thereof.

The methods disclosed herein can further comprise preparing DNA and cDNA libraries from the same sample, such as the same biological sample, comprising: ligating a DNA tag to an end of a DNA molecule in the sample, wherein the DNA tag comprises a UMI and a DNA identifier; and performing reverse transcription of a RNA molecule in the sample in the presence of a RNA tag, wherein the RNA tag comprises a RNA identifier, a UMI, and a poly(T). The reverse transcription can be performed in the presence of a second RNA tag, wherein the second RNA tag comprises a RNA identifier, a UMI, and a template switching oligonucleotide (TSO). The methods can further comprising amplifying the tagged DNA and the tagged cDNA for enrichment with a set of gene specific primers.

The methods can further comprise separating the amplified sample into first, second, or third sample.

The protein and DNA and RNA molecules can be obtained from a biological sample. The DNA and RNA molecules can be fragmented DNA and RNA from the biological sample

The DNA molecule can contain polished ends for ligation. The RNA molecule can be polyadenylated. In some embodiments, the method does not require ribosomal depletion.

The methods can further comprise amplifying the first sample with primers specific for the DNA tag.

The amplification can generate a DNA library corresponding to the DNA in the sample.

The methods can further comprise amplifying the second sample with primers specific for the RNA tag. The amplification can generate a cDNA library corresponding to the RNA in a sample.

The methods can further comprise sequencing the protein-based DNA, DNA, and/or cDNA library.

The DNA molecule can be genomic DNA. The DNA library can be used for DNA variant detection, copy number analysis, fusion gene detection, or structural variant detection.

The cDNA library can be used for RNA variant detection, gene expression analysis, or fusion gene detection. The library can be used for paired DNA and RNA profiling.

In some embodiments, the third oligonucleotide can be separated from the genomic DNA and total RNA.

The methods can further comprise obtaining purified DNA and RNA from the same sample; attaching a DNA tag sequence to the DNA in the sample; attaching an RNA tag sequence to the RNA in the sample; and detecting DNA, RNA, and protein targets, respectively.

The methods disclosed herein can further comprise: (a) obtaining purified DNA and RNA from the same biological sample; (b) fragmenting the DNA and RNA; (c) polishing the ends of the double stranded DNA fragments for ligation; (d) polishing the RNA fragments by polyadenylation; (e) ligating a DNA tag to a 3′ end of the polished DNA fragments, wherein the DNA tag comprises in a 5′ to 3′ direction a unique molecular identifier (UMI) and a DNA identifier; (f) performing reverse transcription of the polished RNA fragments in the presence of a first RNA tag, wherein the first RNA tag comprises in a 5′ to 3′ direction a RNA identifier, a UMI, and a poly(T), and a second RNA tag, wherein the second RNA tag comprises in a 5′ to 3′ direction a RNA identifier, a UMI, and a template switching oligonucleotide (TSO); (g) amplifying the tagged DNA and tagged cDNA for enrichment with a set of gene specific primers; (h) separating the amplified sample into first and second samples; (i) amplifying the first sample with primers specific for the DNA tag; and (j) amplifying the second sample with primers specific for the RNA tag.

Also disclosed herein are protein-based DNA libraries, DNA libraries, and/or cDNA library made by the methods disclosed herein.

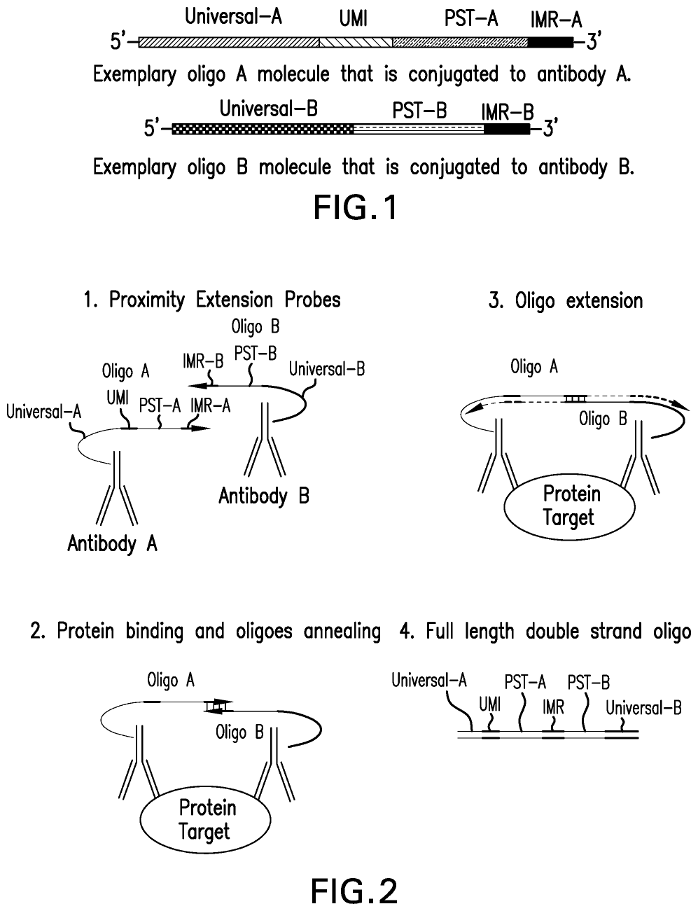

By way of example, a method disclosed herein can use antibody pairs containing two antibodies for a specific protein target. The antibody pair (antibody A and antibody B) can be one polyclonal Ab divided into two, two different polyclonal Abs, two different monoclonal Abs, or the combination of them. Two different oligos are conjugated to the two antibodies respectively, to form a first and second proximity probes. Each oligo (oligo A or oligo B) can comprise a universal amplification region, e.g., for PCR amplification, variable probe specific tag region (PST) for differentiating target protein, UMI region for molecule counting, and inter-molecular reacting region (IMR) for facilitating oligo pair interaction, either by ligation (PLA) or extension (PEA). An exemplary illustration of the oligo pair is shown in FIG. 1.

The UMI can be in both of the oligos in the pair. For example, the UMI can also be included in oligo B molecule in above example. In such case, the combination of UMIs in both oligos is used for counting purpose.

The conjugation of oligo to antibody can be direct linking through chemical bond, or through hybridization to intermediary oligos linked to antibodies, or though other interacting components (e.g., streptavidin and biotin) linked to antibody and oligo respectively.

The conjugated probe pair (antibody A conjugated with oligo A, antibody B conjugated with oligo B) is then used for detecting the abundance of a specific target protein in the sample. Different probe pairs are mixed together, so that multiple protein targets can be detected in single reaction. Depending on the oligo design, the probe pairs can be used in PLA or PEA assay. Specifically, the antibody A and antibody B of the proximity probe pair bind to a single protein target, which brings oligo A and oligo B into close proximity. Oligo A and B then interact with each other to form a new oligo, either through ligation by ligase (PLA) or extension by DNA polymerase (PEA). A demonstration of PEA workflow using proximity probe pair of above oligos is shown FIG. 2. The workflow shows PEA using UMI bearing probes. The free 3′ end is shown with arrow.

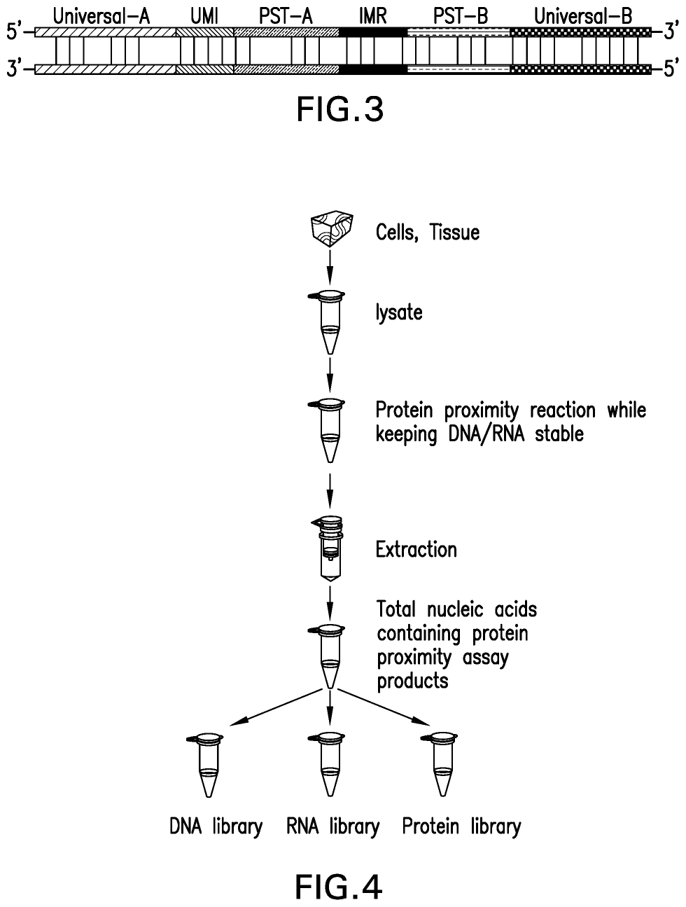

The resulting new oligo, referred to herein as a “third oligonucleotide” or “proximity oligonucleotide,” is composed of universal region on both ends, UMI region, two parts of probe specific tag region (PST-A and PST-B), and inter-molecular reacting region (IMR). It can be either single stranded (PLA or PEA) or double stranded (PEA). An exemplary double stranded oligo from the above PEA assay is shown in FIG. 3.

The third oligonucleotide can be further modified by adding appropriate adapters (either by PCR or ligation), so that they can be analyzed on a NGS platform. From the sequencing reads, the sequence of Universal-A and Universal-B serves as a signature tag signaling that the read is for protein sample. This is particularly helpful if other types of reads from DNA and RNA samples are all to be analyzed in the same platform. The sequence of PST-A+IMR+PST-B uniquely identifies each protein target. UMI counting measures the abundance of the corresponding protein target in the sample.

For example, a typical Illumina Miseq sequencing read can be as follows:

(SEQ ID NO: 1)
CCACTGGGTCTGGTCAATCACGCCNNNNNNNNAACCATTAGCTGACATTC
CGCTCTAGGATCCGGAGTCACCATATCCATAAGATATGAACGCATTGCCC
GGCCCGCTCGATTCCATGAACTTTCCC.

The italic regions are universal sequences. The underlined region (PST-A+IMR+PST-B) uniquely identifies each protein target. The bold region is UMI for counting the abundance of the corresponding protein target in the sample. Compared to the use of read count only, the use of UMI count can effectively offset PCR amplification bias, improving data analysis accuracy. The UMI count for each protein target in a sample is first normalized against the UMI count of the controls. The normalized UMI count can then be compared across different samples. The higher the normalized count, the more abundant the corresponding target is in the sample.

The methods disclosed herein can be incorporated into regular DNAseq and RNAseq workflow, allowing the analysis of protein/DNA/RNA simultaneously, only DNA and RNA simultaneously, or each separately from the same sample. An example workflow is provided in FIG. 4. The separation of DNA products of proximity reaction from genomic DNA and total RNA can ease downstream NGS library preparation. The DNA products of proximity reaction can be separated from genomic DNA, based on their shorter length than gDNA, by simple size selection methods. The proximity oligonucleotides can also contain affinity labels (such as Biotin) to facilitate its separation from genomic DNA and total RNA. See FIG. 4.

Disclosed herein are integrative analyte-based DNA, DNA, and cDNA library preparations for analysis, such as by next-generation sequencing (NGS) analysis, without physical separation of DNA and RNA in the sample. These approaches integrate UMI (unique molecular index) technology and optionally, targeted enrichment technology, seamlessly into the workflow, which improve utilization of sequencing capacity and accuracy of the results. In addition, these methods output three separate analyte-based DNA, DNA and cDNA libraries from analyte, DNA and RNA, respectively, which allow flexible manipulation on downstream sequencing platform. Compared to standalone DNA library and cDNA library methods, these approaches reduce sample consumption, simplify the experimental process, and can help researchers gain biological insights in genotype and phenotype correlations and molecular mechanisms of diseases.

Methods are described herein to prepare targeted DNA and cDNA libraries without the necessity of physical separation of genomic DNA (gDNA) and mRNA. The process involves three modules: (1) assign different DNA and RNA tag molecules to each individual DNA and RNA fragment, respectively, without separating them in the system; optionally, (2) amplify and enrich a subset of the tagged DNA and RNA fragments (target enrichment); and (3) differentially PCR amplify the tagged DNA and tagged cDNA in the (enriched) product to output two libraries corresponding to the original DNA and RNA, respectively.

The DNA and RNA tag molecules used in the first module are oligonucleotides comprising at least 1) an identifying sequence to distinguish a DNA library or RNA library, and 2) a UMI sequence for identifying each individual nucleic acid molecule.

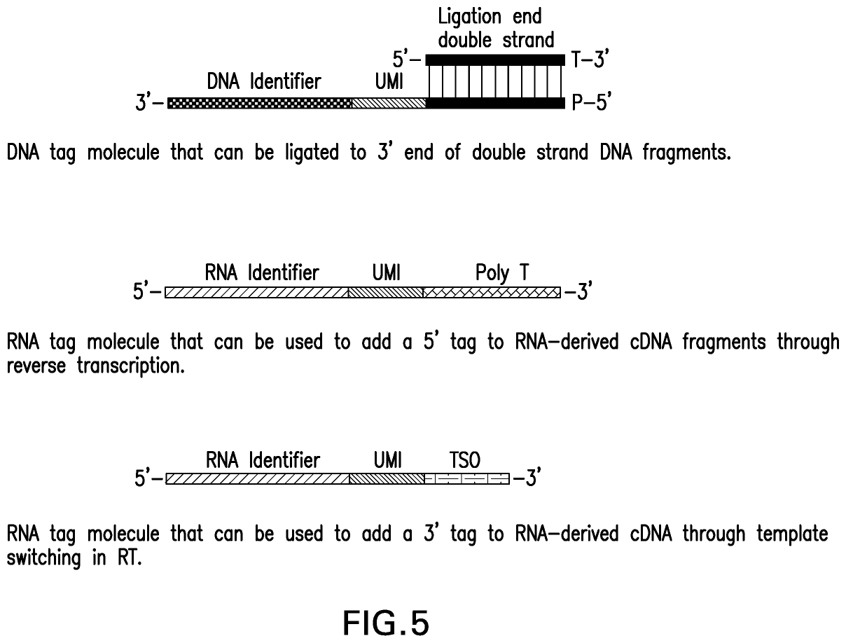

The DNA and RNA tags are essential for the final separation of DNA and cDNA libraries in module 3, where they can serve as specific amplification primer sites for DNA and RNA. The UMI sequence helps improve accuracy for both DNA and RNA NGS analysis. Exemplary tag molecules are illustrated in FIG. 5.

Two types of RNA tag molecules can be used in order to sequence the single stranded RNA from both directions, and thus, two different mechanisms can be used to attach the RNA specific sequence. Only one type of DNA tag molecule is needed because the DNA tag molecule can be ligated to both ends of the double stranded DNA.

The targeted enrichment reaction (module 2) enables focused view on relevant regions of interest and provides economic utilization of NGS sequencing capacity. It also mitigates the necessity for extra treatment of the sample associated with whole genome or transcriptome workflow, such as ribosomal RNA depletion. The enrichment is done in the same reaction for both DNA and RNA. Depending on the applications, the enrichment primer pool can be the same if the target DNA and RNA regions are the same. If different regions are of interest for the DNA and RNA, users can simply mix the corresponding enrichment primer pools, and put them into the same reaction.

Module 3 enables separated output of DNA and cDNA libraries. The sequencing depth requirements for DNA and cDNA are usually quite different, and they vary depending on the applications. The output from the methods disclosed herein gives users flexibility so that sequencing capacity can be allocated individually according to specific needs. In addition, since the samples have already been partially amplified in module 2, the separation has negligible effect on sample loss.

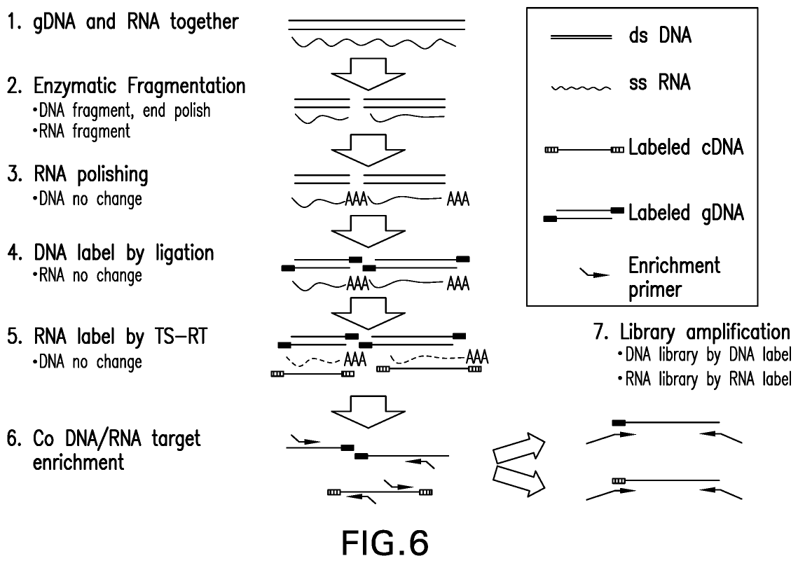

FIG. 6 illustrates one exemplary, optimized way to utilize the methods disclosed herein. It starts with purified (not necessarily separated) gDNA and RNA from a biological sample (step 1). The total nucleic acids are fragmented by enzymatic digestion (for DNA) and by heat hydrolysis (for RNA). The double stranded DNA fragments are end polished so that they are ready for ligation (step 2). The fragmented RNAs are end polished by polyadenylation (step 3). In the next few steps, DNA fragments are ligated to DNA tag molecules (step 4), and the RNA fragments are attached with RNA tag molecules (on both ends) by template switching reverse transcription (step 5). With both DNA and RNA tags in place, the sample is subjected to targeted enrichment reaction by a set of gene specific primers, in which the regions of interest are amplified and enriched (step 6). Finally, the sample is split into two samples, and further amplified by primers specific for the DNA tag and RNA tag, respectively, and with proper NGS adapter sequences compatible with, e.g., Illumina NGS platform (step 7). The final products are two separate DNA and cDNA libraries resulted from the original DNA and RNA material, respectively, and are ready for sequencing.

In addition to preparing an analyte-based DNA library from a sample, disclosed herein are methods for preparing DNA and cDNA libraries from the same sample, comprising: ligating a DNA tag to an end of a DNA molecule in a sample, wherein the DNA tag comprises a unique molecular identifier (UMI) and a DNA identifier; and performing reverse transcription of a RNA molecule in the sample in the presence of a RNA tag, wherein the RNA tag comprises a RNA identifier, a UMI, and a poly(T). The methods do not require physical separation of the DNA and RNA from the sample.

In some embodiments, the reverse transcription is performed in the presence of a second RNA tag, wherein the second RNA tag comprises a RNA identifier, a UMI, and a template switching oligonucleotide (TSO).

In some embodiments, the methods can include ribosomal depletion. Alternatively, in some embodiments, the methods do not require ribosomal depletion. Methods for ribosomal depletion are known in the art, e.g., using RiboZero gold (Illumina: MRZG126).

The term “sample” can include peptides, polypeptides, proteins, RNA, DNA, a single cell, multiple cells, fragments of cells, or an aliquot of body fluid, taken from a subject (e.g., a mammalian subject, an animal subject, a human subject, or a non-human animal subject). Samples can be selected by one of skill in the art using any known means known including but not limited to centrifugation, venipuncture, blood draw, excretion, swabbing, biopsy, needle aspirate, lavage sample, scraping, surgical incision, laser capture microdissection, gradient separation, or intervention or other means known in the art. The term “mammal” or “mammalian” as used herein includes both humans and non-humans and include but is not limited to humans, non-human primates, canines, felines, murines, bovines, equines, and porcines.

As used herein, the term “biological sample” is intended to include, but is not limited to, tissues, cells, biological fluids and isolates thereof, isolated from a subject, as well as tissues, cells, and fluids present within a subject.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

A single cell suspension can be obtained using standard methods known in the art including, for example, enzymatically using trypsin or papain to digest proteins connecting cells in tissue samples or releasing adherent cells in culture, or mechanically separating cells in a sample. Samples can also be selected by one of skill in the art using one or more markers known to be associated with a sample of interest.

Methods for manipulating single cells are known in the art and include fluorescence activated cell sorting (FACS), micromanipulation and the use of semi-automated cell pickers (e.g., the Quixell™ cell transfer system from Stoelting Co.). Individual cells can, e.g., be individually selected based on features detectable by microscopic observation, such as location, morphology, or reporter gene expression.

Once a desired sample has been identified, the sample is prepared and the cell(s) are lysed to release cellular contents including DNA and RNA, such as gDNA and mRNA, using methods known to those of skill in the art. Lysis can be achieved by, for example, heating the cells, or by the use of detergents or other chemical methods, or by a combination of these. Any suitable lysis method known in the art can be used.

Proteins or nucleic acids such as DNA or RNA from a cell are isolated using methods known to those of skill in the art.

As used herein, an “analyte” is any molecule that is to be identified and/or quantified in a sample, such as but not limited to peptides, polypeptides, proteins, antibodies, antigens, ligands, receptors, bacterial or viral components, small molecules, polynucleotides, oligonucleotides, etc. Analytes can include agents such as, e.g., drugs or other compounds administered either to inhibit or to treat or prevent a disorder and/or disease.

In the first and second proximity probes, the first and second analyte binding domains, respectively, can be antibodies, aptamers, ligands, receptors, or a combination thereof that are capable of interacting with analytes of interest.

The terms “polypeptide,” “peptide,” and “protein,” used interchangeably herein, refer to a polymeric form of amino acids of any length. NH₂ refers to the free amino group present at the amino terminus of a polypeptide. COOH refers to the free carboxyl group present at the carboxyl terminus of a polypeptide.

The terms “protein-based DNA” and “analyte-based DNA” refer to a DNA that is associated with a protein or analyte of interest, respectively, due to the interaction of the protein or analyte, respectively, with the analyte binding domain, which in turn is associated with the first and second oligonucleotide domain.

The term “polynucleotide(s)” or “oligonucleotide(s)” refers to nucleic acids such as DNA molecules and RNA molecules and analogs thereof (e.g., DNA or RNA generated using nucleotide analogs or using nucleic acid chemistry). As desired, the polynucleotides can be made synthetically, e.g., using art-recognized nucleic acid chemistry or enzymatically using, e.g., a polymerase, and, if desired, can be modified. Typical modifications include methylation, biotinylation, and other art-known modifications. In addition, a polynucleotide can be single-stranded or double-stranded and, where desired, linked to a detectable moiety. In some aspects, a polynucleotide can include hybrid molecules, e.g., comprising DNA and RNA.

“G,” “C,” “A,” “T” and “U” each generally stands for a nucleotide that contains guanine, cytosine, adenine, thymidine and uracil as a base, respectively. However, it will be understood that the term “ribonucleotide” or “nucleotide” can also refer to a modified nucleotide or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in nucleotide sequences by a nucleotide containing, for example, inosine. In another example, adenine and cytosine anywhere in the oligonucleotide can be replaced with guanine and uracil, respectively, to form G-U Wobble base pairing with the target mRNA. Sequences containing such replacement moieties are suitable for the compositions and methods described herein.

The term “DNA” refers to chromosomal DNA, plasmid DNA, phage DNA, or viral DNA that is single stranded or double stranded. DNA can be obtained from prokaryotes or eukaryotes.

The term “genomic DNA” or gDNA” refers to chromosomal DNA.

The term “messenger RNA” or “mRNA” refers to an RNA that is without introns and that can be translated into a polypeptide.

The term “cDNA” refers to a DNA that is complementary or identical to an mRNA, in either single stranded or double stranded form.

Unique molecular indices or identifiers (UMIs; also called Random Molecular Tags (RMTs)) are short sequences or “barcodes” of bases used to tag each analyte, protein, DNA or RNA molecule (fragment) prior to library amplification, thereby aiding in the identification of each individual nucleic acid molecule, or PCR duplicates. See Kivioj a, T. et al., Nat. Methods 9:72-74 (2012), and Suppl. If two reads align to the same location and have the same UMI, it is highly likely that they are PCR duplicates originating from the same fragment prior to amplification. UMIs can also be used to detect and quantify unique mRNA transcripts. In some embodiments, DNA tags containing the same DNA identifier sequence contain different UMI sequences. In some embodiments, RNA tags containing the same RNA identifier sequence contain different UMI sequences.

A UMI region is used for molecule counting. The concept of UMIs is that prior to any amplification, each original target molecule is ‘tagged’ by a unique barcode sequence. This DNA sequence must be long enough to provide sufficient permutations to assign each founder molecule a unique barcode. In some embodiments, a UMI sequence contains randomized nucleotides and is incorporated into the oligonucleotide domain of the proximity probe, or DNA or RNA tag. For example, a 12-base random sequence provides 4¹² or 16,777,216 UMI's for each target molecule in the sample.

An adapter can be attached to the third oligonucleotide, e.g., by amplification or ligation, to facilitate analysis of the third oligonucleotide by sequencing, such as NGS.

A “variable probe specific tag region” (PST) is a specific sequence used to differentiate the target analyte(s) or protein(s). Due to the interaction of the protein or analyte, respectively, with the analyte binding domain, which in turn is associated with the first and second oligonucleotide domains, the PST sequence on the probe is associated with the corresponding target analyte(s) or protein(s), so that a different PST represents different analyte or protein.

An “inter-molecular reacting region” (IMR) facilitates an oligonucleotide pair interaction, either by ligation (PLA) or extension (PEA). An IMR is a region in the first proximity probe that interacts with the IMR region in the second proximity probe, such as by hybridization. Thus, the IMR of the first proximity probe can be 100%, 99%, 98%, 97%, 96%, 95%, 94%, 93%, 92%, 91%, 90%, 85%, or 80% complementary or any range derivable therefrom to the IMR of the second proximity probe. The IMR can be, e.g., but not limited to, 1-100 nucleotides, 1-90 nucleotides, 1-80 nucleotides, 1-60 nucleotides, 1-50 nucleotides, 1-40 nucleotides, 1-30 nucleotides, 1-20 nucleotides, 1-10 nucleotides, or any lengths or ranges derivable therefrom.

The terms “universal PCR handle,” “universal PCR sequence,” “PCR handle,” “PCR handle sequence,” “universal PCR handle,” and “universal amplification sequence” refer to a common nucleic acid sequence useful for enabling amplification, such as PCR amplification, and further sequencing of nucleic acid sequences extracted or derived from the biological units. In some embodiments, the PCR handle lacks homology with the template sequence. In other embodiments, the PCR handle sequence is common for the entire sample preparation workflow. The RNA can be reverse transcribed to cDNA and a template switching oligonucleotide (TSO) can be used to introduce a PCR handle downstream of the synthesized cDNA (Zhu, Y. Y. et al., Biotechniques 30:892-7 (2001), i.e., to append a PCR handle to the 5′ end of full-length cDNAs. The PCR handle is used for subsequent amplification. In some embodiments, having a PCR handle at both the 5′ and 3′ ends, i.e., 2 PCR handles, can increase amplification efficiency.

As used herein, “polymerase” and its derivatives, generally refers to any enzyme that can catalyze the polymerization of nucleotides (including analogs thereof) into a nucleic acid strand. Typically but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer based polymerase that optionally can be reactivated.

The term “extension” and its variants, as used herein, when used in reference to a given primer, comprises any in vivo or in vitro enzymatic activity characteristic of a given polymerase that relates to polymerization of one or more nucleotides onto an end of an existing nucleic acid molecule. Typically but not necessarily such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

As used herein, the terms “ligating,” “ligation,” and their derivatives refer generally to the act or process for covalently linking two or more molecules together, for example, covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the litigation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′phosphate to a 3′ hydroxyl between adjacent nucleotides can be employed. In an exemplary embodiment, an enzyme such as a ligase can be used. Generally for the purposes of this disclosure, an amplified target sequence can be ligated to an adapter to generate an adapter-ligated amplified target sequence.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases can include, but not limited to, T4 DNA ligase, T4 RNA ligase, and E. coli DNA ligase.

As used herein, “ligation conditions” and its derivatives, generally refers to conditions suitable for ligating two molecules to each other. In some embodiments, the ligation conditions are suitable for sealing nicks or gaps between nucleic acids. As defined herein, a “nick” or “gap” refers to a nucleic acid molecule that lacks a directly bound 5′ phosphate of a mononucleotide pentose ring to a 3′ hydroxyl of a neighboring mononucleotide pentose ring within internal nucleotides of a nucleic acid sequence. As used herein, the term nick or gap is consistent with the use of the term in the art. Typically, a nick or gap can be ligated in the presence of an enzyme, such as ligase at an appropriate temperature and pH. In some embodiments, T4 DNA ligase can join a nick between nucleic acids at a temperature of about 70-72° C.

As used herein, “blunt-end ligation” and its derivatives, refers generally to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang.” In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. Typically, blunted-end ligation excludes the use of additional oligonucleotide adapters to assist in the ligation of the double-stranded amplified target sequence to the double-stranded adapter, such as patch oligonucleotides as described in Mitra and Varley, US2010/0129874. In some embodiments, blunt-ended ligation includes a nick translation reaction to seal a nick created during the ligation process.

The term “amplicon” refers to the amplified product of a nucleic acid amplification reaction, e.g., RT-PCR.

The terms “reverse-transcriptase PCR” and “RT-PCR” refer to a type of PCR where the starting material is mRNA. The starting mRNA is enzymatically converted to complementary DNA or “cDNA” using a reverse transcriptase enzyme. The cDNA is then used as a template for a PCR reaction.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000), each of which are hereby incorporated by reference in their entireties.

The term “hybridize” refers to a sequence specific non-covalent binding interaction with a complementary nucleic acid. Hybridization can occur to all or a portion of a nucleic acid sequence. Those skilled in the art will recognize that the stability of a nucleic acid duplex, or hybrids, can be determined by the Tm. Additional guidance regarding hybridization conditions can be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

As used herein, “incorporating” a sequence into a polynucleotide refers to covalently linking a series of nucleotides with the rest of the polynucleotide, for example at the 3′ or 5′ end of the polynucleotide, by phosphodiester bonds, wherein the nucleotides are linked in the order prescribed by the sequence. A sequence has been “incorporated” into a polynucleotide, or equivalently the polynucleotide “incorporates” the sequence, if the polynucleotide contains the sequence or a complement thereof. Incorporation of a sequence into a polynucleotide can occur enzymatically (e.g., by ligation or polymerization) or using chemical synthesis (e.g., by phosphoramidite chemistry).

As used herein, the terms “amplify” and “amplification” refer to enzymatically copying the sequence of a polynucleotide, in whole or in part, so as to generate more polynucleotides that also contain the sequence or a complement thereof. The sequence being copied is referred to as the template sequence. Examples of amplification include DNA-templated RNA synthesis by RNA polymerase, RNA-templated first-strand cDNA synthesis by reverse transcriptase, and DNA-templated PCR amplification using a thermostable DNA polymerase. Amplification includes all primer-extension reactions. Amplification includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting amplification reaction are commercially available. Primers useful to amplify sequences from a particular gene region are preferably complementary to, and hybridize specifically to sequences in the target region or in its flanking regions and can be prepared using the polynucleotide sequences provided herein. Nucleic acid sequences generated by amplification can be sequenced directly.

When hybridization occurs in an antiparallel configuration between two single-stranded polynucleotides, the reaction is called “annealing” and those polynucleotides are described as “complementary”. As used herein, and unless otherwise indicated, the term “complementary,” when used to describe a first nucleotide sequence in relation to a second nucleotide sequence, refers to the ability of a polynucleotide comprising the first nucleotide sequence to hybridize and form a duplex structure under certain conditions with a polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences include base-pairing of a region of a polynucleotide comprising a first nucleotide sequence to a region of a polynucleotide comprising a second nucleotide sequence over the length or a portion of the length of one or both nucleotide sequences. Such sequences can be referred to as “complementary” with respect to each other herein. However, where a first sequence is referred to as “substantially complementary” with respect to a second sequence herein, the two sequences can be complementary, or they can include one or more, but generally not more than about 5, 4, 3, or 2 mismatched base pairs within regions that are base-paired. For two sequences with mismatched base pairs, the sequences will be considered “substantially complementary” as long as the two nucleotide sequences bind to each other via base-pairing.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

In some embodiments, the double stranded DNA fragments can be end polished so that they are amenable for ligation. For example, the ends of the DNA fragments can be polished to have blunt ends. As known in the art, this can be achieved with enzymes that can either fill in or remove the protruding strand. Another method is to perform the ligation in the presence of short synthetic oligonucleotides, called “adapters,” which have been prepared in such a way as to eventually ligate with one terminus to the fragment and make the fragment amenable for ligation with polynucleotides of interest such as DNA or RNA tags. As such, the DNA fragments can be ligated to DNA tags.

In some embodiments, the RNA fragments are end polished by polyadenylation. The RNA fragments can be attached to RNA tags, e.g., on both ends, by template switching reverse transcription.

A “DNA tag” or “DNA tag molecule” is a polynucleotide comprising a DNA identifier and a UMI. A DNA tag can be a deoxyribopolynucleotide. A “DNA identifier” is a polynucleotide sequence assigned to distinguish a gDNA molecule from a RNA molecule. A DNA tag can be ligated to the 5′ or 3′ end of double stranded DNA fragments.

A “RNA tag” or “RNA tag molecule” is a polynucleotide comprising a RNA identifier and a UMI. A RNA tag can be a deoxyribopolynucleotide. A “RNA identifier” is a polynucleotide sequence assigned to distinguish a cDNA molecule from a gDNA molecule. A RNA tag can further comprise poly(T). Alternatively, a RNA tag can further comprise a template switching oligonucleotide (TSO). A RNA tag can be used to add a 5′ tag to RNA-derived cDNA fragments through reverse transcription. In some embodiments, a RNA tag can be used to add a 3′ tag to RNA-derived cDNA through template switching in reverse transcription.

Two types of RNA tags are helpful because in order to sequence the single stranded RNA from both directions, two different mechanisms can be used to attach the RNA specific sequence. Only one type of DNA tag is needed because the DNA tag can be ligated to both ends of the double stranded DNA.

A composition can comprise at least 2 of the tags described above, e.g., a DNA tag and a RNA tag. A composition can also comprise the 3 tags described above, e.g., a DNA tag and the 2 types of RNA tags.

In some embodiments, the RNA tag is a single-stranded DNA molecule and serves as a primer for reverse transcription. The RNA tag can be generated using a DNA polymerase (DNAP). Here, the binding site of the RNA tag is an RNA binding site (e.g., an mRNA binding site) and contains a sequence region complementary to a sequence region in one or more RNAs. In some embodiments, the binding site is complementary to a sequence region common to all RNAs in the sample to which the barcode adapter is added. For example, the binding site can be a poly(T) tract, which is complementary to the poly(A) tails of eukaryotic mRNAs. Alternatively or in addition, the binding site can include a random sequence tract. Upon adding the RNA tag to the RNAs associated with a sample, reverse transcription can occur and first strands of cDNA can be synthesized, such that the RNA identifier sequence is incorporated into the first strands of cDNA. It will be recognized that reverse transcription requires appropriate conditions, for example the presence of an appropriate buffer and reverse transcriptase enzyme, and temperatures appropriate for annealing of the barcode adapter to RNAs and the activity of the enzyme. It will also be recognized that reverse transcription, involving a DNA primer and an RNA template, is most efficient when the 3′ end of the primer is complementary to the template and can anneal directly to the template. Accordingly, the RNA tag can be designed so that the binding site occurs at the 3′ end of the adapter molecule.

As described above, the present methods can employ a reverse transcriptase enzyme that adds one or more non-templated nucleotides (such as Cs) to the end of a nascent cDNA strand upon reaching the 5′ end of the template RNA. These nucleotides form a 3′ DNA overhang at one end of the RNA/DNA duplex. If a second RNA molecule contains a sequence region, for example, a poly-G tract at its 3′ end that is complementary to the non-templated nucleotides, and binds to the non-templated nucleotides, the reverse transcriptase can switch templates and continue extending the cDNA, now using the second RNA molecule as a template. Such a second RNA molecule is referred to herein and known in the art as a template-switching oligo (TSO).

In embodiments of the present methods, a second RNA tag comprising a RNA identifier, UMI, and TSO can serve as a template-switching oligonucleotide for reverse transcription. Thus, the RNA identifier sequence is incorporated into the first strand of cDNA after template switching, and is present in DNA molecules resulting from amplification (for example, by PCR) of the first strand of cDNA. In these embodiments, any reverse transcriptase that has template switching activity can be used. The binding site of the first RNA tag is a cDNA binding site and preferably occurs at the 3′ end of the adapter molecule. The binding site can include a G-tract (comprising one or more G nucleotides), or any other sequence that is at least partially complementary to that of the 3′ overhang generated by the reverse transcriptase. It will be recognized that the overhang sequence, and thus an appropriate sequence for the binding site of the barcode adapter, can depend on the choice of reverse transcriptase used in the method.

Methods for reverse transcription and template switching are well known in the art. A procedure frequently referred to as “SMART” (switching mechanism at the 5′ end of the RNA transcript) can generate full-length cDNA libraries, even from single-cell-derived RNA samples. This strategy relies on the intrinsic properties of Moloney murine leukemia virus (MMLV) reverse transcriptase and the use of a unique template switching oligonucleotide (TS oligo, or TSO). Moloney Murine Leukemia Virus Reverse Transcriptase (M-MLV RT) is an RNA-dependent DNA polymerase that can be used in cDNA synthesis with long messenger RNA templates (>5 kb). The enzyme is a product of the pol gene of M-MLV and consists of a single subunit with a molecular weight of 71 kDa. During first-strand synthesis, upon reaching the 5′ end of the RNA template, the terminal transferase activity of the MMLV reverse transcriptase adds a few additional nucleotides (mostly deoxycytidine) to the 3′ end of the newly synthesized cDNA strand. These bases function as a TS oligo-anchoring site. Upon base pairing between the TS oligo and the appended deoxycytidine stretch, the reverse transcriptase “switches” template strands, from cellular RNA to the TS oligo, and continues replication to the 5′ end of the TS oligo. By doing so, the resulting cDNA contains the complete 5′ end of the transcript, and universal sequences of choice can be added to the reverse transcription product. Along with tagging of the cDNA 3′ end by oligo dT primers, this approach makes it possible to efficiently amplify the entire full-length transcript pool in a completely sequence-independent manner.

A TS oligo can be a DNA oligo sequence that carries 3 riboguanosines (rGrGrG) at its 3′ end. The complementarity between these consecutive rG bases and the 3′ dC extension of the cDNA molecule allows the subsequent template switching. The 3′ most rG can also be replaced with a locked nucleic acid base (LNA) to enhance thermostability of the LNA monomer, which would be advantageous for base pairing.

The TSO can include a 3′ portion comprising a plurality of guanosines or guanosine analogues that base pair with cytosine. Non-limiting examples of guanosines or guanosine analogues useful in the methods described herein include, but are not limited to, deoxyriboguanosine, riboguanosine, locked nucleic acid-guanosine, and peptide nucleic acid-guanosine. The guanosines can be ribonucleosides or locked nucleic acid monomers.

The TSO can include a 3′ portion including at least 2, at least 3, at least 4, at least 5, or 2, 3, 4, or 5, or 2-5 guanosines, or guanosine analogues that base pair with cytosine. The presence of a plurality of guanosines (or guanosine analogues that base pair with cytosine) allows the TSO to anneal transiently to the exposed cytosines at the 3′ end of the first strand of cDNA. This causes the reverse transcriptase to switch template and continue to synthesis a strand complementary to the TSO. In one aspect of the invention, the 3′ end of the TSO can be blocked, for example by a 3′ phosphate group, to prevent the TSO from functioning as a primer during cDNA synthesis.

Before the tagged cDNA samples are pooled, synthesis of cDNA can be stopped, for example by removing or inactivating the reverse transcriptase. This prevents cDNA synthesis by reverse transcription from continuing in the pooled samples.

As used herein, “amplified target sequences” and its derivatives, refers generally to a nucleic acid sequence produced by the amplification of/amplifying the target sequences using target-specific primers and the methods provided herein. The amplified target sequences can be either of the same sense (the positive strand produced in the second round and subsequent even-numbered rounds of amplification) or antisense (i.e., the negative strand produced during the first and subsequent odd-numbered rounds of amplification) with respect to the target sequences. For the purposes of this disclosure, the amplified target sequences are typically less than 50% complementary to any portion of another amplified target sequence in the reaction.

The term “polymerase chain reaction” (“PCR”) of Mullis (U.S. Pat. Nos. 4,683,195, 4,683,202, and 4,965,188) refers to a method for increasing the concentration of a segment of a target sequence in a mixture of nucleic acid sequences without cloning or purification. This process for amplifying the target sequence consists of introducing a large excess of two oligonucleotide primers to the nucleic acid sequence mixture containing the desired target sequence, followed by a precise sequence of thermal cycling in the presence of a polymerase (e.g., DNA polymerase). The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

The methods disclosed herein can further comprise amplifying the tagged DNA the tagged cDNA for enrichment with a set of gene specific primers. Target enrichment can be achieved with, e.g., an SPE primer pool, DNA boosting primer, and RNA boosting primer. Amplicon-based next-generation sequencing (NGS) assays offer many advantages for targeted enrichment. For example, QIAseq NGS panels employ unique molecular indices (UMI's) to correct for PCR amplification bias and use single primer extension (SPE) technology which provides design flexibility and highly-specific target enrichment. The concept of UMIs is that prior to any amplification, each original target molecule is ‘tagged’ by a unique barcode sequence. This DNA sequence must be long enough to provide sufficient permutations to assign each founder molecule a unique barcode. In its current form, a 12-base random sequence provides 4¹² or 16,777,216 UMI's for each target molecule in the sample.

As used herein, the term “primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate in sequence. Primers within the scope of the present invention bind adjacent to a target sequence. A “primer” can be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 17 to 30 nucleotides. In some embodiments, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

As used herein, “target-specific primer” and its derivatives, refers generally to a single-stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or 100% identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or 100% identical, across its entire length to at least a portion of a nucleic acid molecule that includes its corresponding target sequence. In some embodiments, a target-specific primer can be at least 90%, at least 95% complementary, at least 98% complementary or at least 99% complementary, or 100% identical, across its entire length to at least a portion of its corresponding target sequence. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer can be substantially non-complementary at its 3′ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′ end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

Primer design is based on single primer extension, in which each genomic target is enriched by one target-specific primer and one universal primer—a strategy that removes conventional two target-specific primer design restriction and reduces the amount of required primers. All primers required for a panel are pooled into an individual primer pool to reduce panel handling and the number of pools required for enrichment and library construction.

The booster panel is a pool of up to 100 primers that can be used to boost the performance of certain primers in any panel (cataloged, extended, or custom), or to extend the contents of an existing custom panel. The primers are delivered as a single pool that can be spiked into the existing panel.

After removing unused adapters, a limited number of PCR cycles can be conducted using an adapter primer and a pool of single primers, each carrying a gene specific sequence and a 5′ universal sequence. During this process, each single primer repeatedly samples the same target locus from different DNA templates. Afterwards, additional PCR cycles can be conducted using universal primers to attach complete adapter sequences and to amplify the library to the desired quantity.

Compared to existing targeted enrichment approaches, the SPE method relies on single end adapter ligation, which inherently has a much higher efficiency than requiring adapters to ligate to both ends of the dsDNA fragment. More DNA molecules will be available for the downstream PCR enrichment step. PCR enrichment efficiency using one primer is also better than conventional two primer approach, due to the absence of an efficiency constraint from a second primer. During the initial PCR cycles, primers have repeated opportunities to convert (i.e. capture) maximal amount of original DNA molecules into amplicons.

All three features help to increase the efficiency of capturing rare mutations in the sample. In addition, incorporated UMI's within the amplicon are the key to estimating the number of DNA molecules captured and to greatly reduce sequencing errors in downstream analysis. Single primer extension also permits discovery of unknown structural variants, such as gene fusions.

The targeted enriched sample of DNA (e.g., gDNA) and cDNA are split into 2 separate samples. A first sample can be amplified by polymerase chain reaction (PCR) using primers specific for the DNA tag to generate a DNA library corresponding to the DNA in the sample. A second sample can be amplified by PCR using primers specific for the RNA tag to generate a cDNA library corresponding to the RNA in the sample.

A real-time polymerase chain reaction (Real-Time PCR), also known as quantitative polymerase chain reaction (qPCR), is a laboratory technique of molecular biology based on the polymerase chain reaction (PCR). It monitors the amplification of a targeted DNA molecule during the PCR, i.e. in real-time, and not at its end, as in conventional PCR. Real-time PCR can be used quantitatively (quantitative real-time PCR), and semi-quantitatively, i.e. above/below a certain amount of DNA molecules (semi quantitative real-time PCR). Other types of PCRs include but are not limited to nested PCR (used to analyze DNA sequences coming from different organisms of the same species but that can differ for a single nucleotide (SNIPS) and to ensure amplification of the sequence of interest in each of the organism analyzed) and Inverse-PCR (usually used to clone a region flanking an insert or a transposable element).

Two common methods for the detection of PCR products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary sequence.

Methods and kits for performing PCR are well known in the art. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press).

Embodiments of the invention provide 2 separate libraries for flexible manipulation downstream: a DNA library based on the original DNA and a cDNA library based on the original RNA produced by any of the methods described herein. The DNA library or cDNA library can be sequenced to provide an analysis of gene expression in single cells or in a plurality of single cells.

The amplified DNA or cDNA library can be sequenced and analyzed using methods known to those of skill in the art, e.g., by next-generation sequencing (NGS). In certain exemplary embodiments, RNA expression profiles are determined using any sequencing methods known in the art. Determination of the sequence of a nucleic acid sequence of interest can be performed using a variety of sequencing methods known in the art including, but not limited to, sequencing by synthesis (SBS), sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (US2009/0018024), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLiD, Helicos, Complete Genomics, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

Embodiments of the invention also provide methods for analyzing gene expression in a plurality of single cells, the method comprising the steps of preparing a cDNA library using the method described herein and sequencing the cDNA library. A “gene” refers to a polynucleotide containing at least one open reading frame (ORF) that is capable of encoding a particular polypeptide or protein after being transcribed and translated. Any of the polynucleotide sequences described herein can be used to identify larger fragments or full-length coding sequences of the gene with which they are associated. Methods of isolating larger fragment sequences are known to those of skill in the art.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

The cDNA library can be sequenced by any suitable screening method. In particular, the cDNA library can be sequenced using a high-throughput screening method, such as Applied Biosystems' SOLiD sequencing technology, or Illumina's Genome Analyzer. In one aspect of the invention, the cDNA library can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

The DNA or gDNA library generated by the methods disclosed herein can be useful for, but not limited to, DNA variant detection, copy number analysis, fusion gene detection and structural variant detection. The cDNA library generated by the methods disclosed herein can be useful for, but not limited to, RNA variant detection, gene expression analysis, and fusion gene detection. The protein-based DNA, DNA and cDNA libraries can also be used for paired protein, DNA, and RNA profiling.

The expression profiles described herein are useful in the field of predictive medicine in which diagnostic assays, prognostic assays, pharmacogenomics, and monitoring clinical trials are used for prognostic (predictive) purposes to thereby treat an individual prophylactically. Accordingly, some embodiments relate to diagnostic assays for determining the expression profile of nucleic acid sequences (e.g., proteins or RNAs), in order to determine whether an individual is at risk of developing a disorder and/or disease. Such assays can be used for prognostic or predictive purposes to thereby prophylactically treat an individual prior to the onset of the disorder and/or disease. Accordingly, in certain exemplary embodiments, methods of diagnosing and/or prognosing one or more diseases and/or disorders using one or more of expression profiling methods described herein are provided.

Some embodiments pertain to monitoring the influence of agents (e.g., drugs or other compounds administered either to inhibit or to treat or prevent a disorder and/or disease) on the expression profile of nucleic acid sequences (e.g., proteins or RNAs) in clinical trials. Accordingly, in certain exemplary embodiments, methods of monitoring one or more diseases and/or disorders before, during and/or subsequent to treatment with one or more agents using one or more of expression profiling methods described herein are provided.

Monitoring the influence of agents (e.g., drug compounds) on the level of expression of a marker of the invention can be applied not only in basic drug screening, but also in clinical trials. For example, the effectiveness of an agent to affect an expression profile can be monitored in clinical trials of subjects receiving treatment for a disease and/or disorder associated with the expression profile. In certain exemplary embodiments, the methods for monitoring the effectiveness of treatment of a subject with an agent (e.g., an agonist, antagonist, peptidomimetic, protein, peptide, nucleic acid, small molecule, or other drug candidate) comprising: (i) obtaining a pre-administration sample from a subject prior to administration of the agent; (ii) detecting one or more expression profiled in the pre-administration sample; (iii) obtaining one or more post-administration samples from the subject; (iv) detecting one or more expression profiles in the post-administration samples; (v) comparing the one or more expression profiled in the pre-administration sample with the one or more expression profiles in the post-administration sample or samples; and (vi) altering the administration of the agent to the subject accordingly.

The expression profiling methods described herein allow the quantitation of gene expression. Thus, not only tissue specificity, but also the level of expression of a variety of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In other embodiments, the time course of expression of one or more nucleic acid sequences (e.g., genes, mRNAs and the like) in an expression profile can be monitored. This can occur in various biological contexts, as disclosed herein, for example development of a disease and/or disorder, progression of a disease and/or disorder, and processes, such a cellular alterations associated with the disease and/or disorder.

The expression profiling methods described herein are also useful for ascertaining the effect of the expression of one or more nucleic acid sequences (e.g., genes, mRNAs and the like) on the expression of other nucleic acid sequences (e.g., genes, mRNAs and the like) in the same cell or in different cells. This provides, for example, for a selection of alternate molecular targets for therapeutic intervention if the ultimate or downstream target cannot be regulated.

The expression profiling methods described herein are also useful for ascertaining differential expression patterns of one or more nucleic acid sequences (e.g., genes, mRNAs and the like) in normal and abnormal cells. This provides a battery of nucleic acid sequences (e.g., genes, mRNAs and the like) that could serve as a molecular target for diagnosis or therapeutic intervention.

The methods described herein can be used to detect or measure analytes, such as but not limited to protein biomarkers in translational research. Moreover, being able to analyze nucleic acid and protein or analytes on the same platform would significantly reduce the analysis time and provide more insights.

### EXAMPLES

### Example 1

The following is an illustrative example showing how the method described herein can be used for protein analysis. A total of 96 probe pairs are designed to detect 96 different protein targets. Four of them are controls for data normalization purpose. Control 1 and control 2 are for exogenous protein targets not in test samples. The 5′ ends of all the oligos are conjugated to their respective antibodies. Control 3 is extension control in which both oligo A and oligo B are conjugated to the same antibody, so that the extension is independent of antigen binding. Control 4 is detection control to monitor PCR amplification variation, in which the complete full-length oligo is directly spiked into the reaction.

### Example 2

Sample:

1. Human serum sample
2. Human serum sample plus spike-in 5 ng/ml of protein target #1 and #2
3. PBS (negative control)

Antibody Binding:

| | |
|---|---|
| Sample | 1 uL |
| Incubation Solution | 2.1 |
| Incubation Stabalizer | 0.3 |
| Probe A set | 0.3 |
| Probe B set | 0.3 |
| Total volume: | 4 uL |

Incubate at 4° C. overnight (16 hrs).

Extension:

| | |
|---|---|
| Sample from previous step | 4 uL |
| H2O | 85.3 |
| PEA Solution | 10 |
| PEA Enzyme | 0.5 |
| PEA Polymerase | 0.2 |
| Total volume: | 100 uL |

Incubate in thermocycler with heated lid on for 50° C. 20 min→95° C. 5 min→17 cycles of (95° C. 30 sec→54° C. 1 min→60° C. 1 min)→4° C. hold.

Library Amplification:

| Sample from previous step | 1 | uL final conc. | |
|---|---|---|---|
| 5x V2 Buffer | 5 | 1x | |
| 2 mM each dNTP mix | 2.5 | 0.2 mM each | |
| 4 uM IL2PEAFwd Universal primer | 2.5 | 400 | nM |
| 4 uM IL1_id(x)_PEARev Universal Primer | 2.5 | 400 | nM |
| H2O | 10.5 | | |
| Hot-Star Taq Polymerase (6 U/uL) | 1 | 0.24 | U/uL |
| Total volume: | 25 uL | | |

Incubate in thermocycler with heated lid on for 95° C. 13 min→98° C. 2 min→20 cycles of (98° C. 15 sec→60° C. 2 min)→72° C. 5 min→4° C. hold.

Purification: Add 75 uL of ice-cold water to each of the 25 uL sample from previous step to make 100 uL total. Do 1 round of 1.2× Ampure XP beads purification (elute in 20 uL water).

Library Quantification is performed using Agilent Bioanalyzer High Sensitivity DNA chip: Dilute the purified libraries to 2 ng/uL. Load 1 uL of this diluted sample on the bioanalyzer. Obtain molar concentration of the libraries based on bioanalyzer's electropherogram. The libraries are ready for sequencing.

### Example 3

Starting Material: Purified genomic DNA and total RNA. For example, 50 ng gDNA and 50 ng total RNA was purified from THP-1 cell line. Ideally, the relative amount of gDNA and RNA should represent the content in the sample.

DNA/RNA Fragmentation:

| | uL | final conc. | |
|---|---|---|---|
| DNA/RNA sample | X | | |
| H₂O | 11.8 − x | | |
| 10x Fragmentation Buffer | 2 | 1x | |
| 100 mM dATP | 0.6 | 3 | mM |
| Exonuclease I (20 U/uL) | 1.6 | 1.6 | U/uL |
| 5x Fragmentation Enzyme Mix | 4 | 1x | |
| Total volume: | 20 uL | | |

Incubate in thermocycler with heated lid on for 4° C. 1 min → 32° C. 15 min → 75° C. 10 min → 80° C. 20 min → 4° C. hold

RNA Polyadenylation:

| | uL | final conc. | |
|---|---|---|---|
| Sample from previous step | 20 | | |
| H₂O | 0.5 | | |
| 10 mM ATP | 1.25 | 0.5 | mM |
| 10 mM 3′-dATP (blocker) | 1.25 | 0.5 | mM |
| T4 Polynucleotide Kinase (10 U/uL) | 1 | 0.4 | U/uL |
| E. coli Poly(A) Polymerase (5 U/uL) | 1 | 0.2 | U/uL |
| Total volume: | 25 uL | | |

Incubate in thermocycler with heated lid on for 4° C. 1 min → 30° C. 10 min → 4° C. hold

DNA Ligation:

| | uL | final conc. | |
|---|---|---|---|
| Sample from previous step | 25 | | |
| 5x Ligation Buffer | 10 | 1x | |
| 50 uM DNA ligation Adapter | 2.8 | 2.8 | uM |
| 50% PEG-6000 | 7.2 | 7.2% | |
| T4 DNA ligase (600 U/uL) | 5 | 60 | U/uL |
| Total volume: | 50 uL | | |

Incubate in thermocycler with heated lid OFF for 4 C. 1 min → 20 C. 15 min → 4 C. hold

Purification: Add 50 uL of ice-cold water to the 50 uL sample from previous step to make 100 uL total. Do 2 rounds of 1.2× Ampure XP beads purification following manufacturer's manual with the following exceptions: 1st round elution in 52 uL water; and 2nd round elution in 13 uL water.

Reverse Transcription:

| | uL | final conc. | |
|---|---|---|---|
| Sample from previous step | 12.87 | | |
| 7.5 uM TSON10T18NV oligo | 1 | 300 | nM |
| 25 uM TSON10forTS oligo | 1 | 1 | uM |
| 5x SuperScript II Buffer | 5 | 1x | |
| 25 mM each dNTP mix | 1 | 1 mM each | |
| 0.1M DTT | 1.25 | 5 | mM |
| RNase Inhibitor (40 U/uL) | 0.63 | 1 | U/uL |
| 300 mM MgCl₂ | 0.5 | 6 | mM |
| 150 mM MnCl₂ | 0.5 | 3 | mM |
| MMLV Reverse Trancriptase RNase H- (200 U/uL) | 1.25 | 10 | U/uL |

Total volume: 25 uL
Incubate in thermocycler with heated lid on for 4° C. 1 min → 25° C. 10 min → 42° C. 45 min → 70° C. 15 min → 4° C. hold

Purification: Add 75 uL of ice-cold water to the 25 uL sample from previous step to make 100 uL total. Do 1 round of 1.2× Ampure XP beads purification following manufacturer's manual and elute in 16.8 uL water.

Target Enrichment:

| | uL | final conc. | |
|---|---|---|---|
| Sample from previous step | 16.8 | | |
| 5x V2 Buffer | 8 | 1x | |
| 2 mM each dNTP mix | 4 | 0.2 mM each | |
| 100 nM each SPE primer pool | 8 | 20 nM each | |
| 10 uM DNA boosting primer | 0.8 | 400 | nM |
| 10 uM RNA boosting primer | 0.8 | 400 | nM |
| Hot-Star Taq Polymerase (6 U/uL) | 1.6 | 0.24 | U/uL |

Total volume: 40 uL

Incubate in thermocycler with heated lid on for 95° C. 13 min → 98° C. 2 min → 8 cycles of (98° C. 15 sec → 68° C. 10 min) → 72° C. 5 min → 4° C. hold

Purification: Add 60 uL of ice-cold water to the 40 uL sample from previous step to make 100 uL total. Do double size selection 0.5×/0.5× with Ampure XP beads following manufacturer's manual and elute in 22 uL water.

qPCR (Real-Time) to Determine Final Amplification Cycles:

| | For DNA library | | | For RV library | | |
|---|---|---|---|---|---|---|
| | uL | final conc. | | uL | final conc. | |
| Sample from previous step | 2 | | | 2 | | |
| 5x V2 Buffer | 2 | 1x | | 2 | 1x | |
| 2 mM each dNTP mix | 1 | 0.2 mM each | | 1 | 0.2 mM each | |
| H₂O | 2.1 | | | 2.1 | | |
| 20x EveGreen Dye | 0.5 | 1x | | 0.5 | 1x | |
| 4 uM IL2N5RS2 Universal primer | 1 | 400 | nM | 1 | 400 | nM |
| 4 uM DNA Universal Primer | 1 | 400 | nM | 0 | 0 | |
| 4 uM RNA Universal Primer | 0 | 0 | | 1 | 400 | nM |
| Hot-Star Taq Polymerase (6 U/uL) | 0.4 | 0.24 | U/uL | 0.4 | 0.24 | U/uL |

| | |
|---|---|
| Total volume: 10 uL | Total volume: 10 uL |

Run on ABI 7900 real time instrument: 95° C. 13 min → 98° C. 2 min → 30 cycles of (98° C. 15 sec → 62° C. 2 min).
Record the counts for both samples

Universal PCR:

| | For DNA library | | | For RNA library | | |
|---|---|---|---|---|---|---|
| | uL | final conc. | | uL | final conc. | |
| Sample from Target Enrichment | 9 | | | 9 | | |
| 5x V2 Buffer | 5 | 1x | | 5 | 1x | |
| 2 mM each dNTP mix | 2.5 | 0.2 mM each | | 2.5 | 0.2 mM each | |
| 4 uM IL2N5RS2 Universal primer | 2.5 | 400 | nM | 2.5 | 400 | nM |
| 4 uM DNA Universal Primer | 2.5 | 400 | nM | 0 | 0 | |
| 4 uM RNA Universal Primer | 0 | 0 | | 2.5 | 400 | nM |
| H₂O | 2.5 | | | 2.5 | | |
| Hot-Star Taq Polymerase (6 U/uL) | 1 | 0.24 | U/uL | 1 | 0.24 | U/uL |
| | Total volume: | 25 uL | | Total volume: | 25 uL | |

Incubate in thermocycler with heated lid on for 95° C. 13 min → 98° C. 2 min → “X” cycles of (98° C. 15 sec → 62° C. 2 min) → 72° C. 5 min → 4° C. hold
(X = Ct + 4) for DNA sample and RNA sample respectively. For example, if Ct = 19 for DNA, and 15 for RNA, then run 23 cycles for DNA, and 19 cycles for RNA

Purification: Add 75 uL of ice-cold water to each of the 25 uL sample from previous step to make 100 uL total. Do 1 round of 1.2× Ampure XP beads purification following manufacturer's manual and elute in 20 uL water.

Library Quantification using Agilent Bioanalyzer High Sensitivity DNA chip: Dilute the purified libraries to 2 ng/uL. Load 1 uL of this diluted sample on the bioanalyzer. Obtain molar concentration of the libraries based on bionanlyzer's electropherogram. The libraries are ready for sequencing.

Following the workflow, with 50 ng gDNA and 50 ng total RNA input, we obtained 675 ng of DNA library and 455 ng of RNA library. The same amount of 50 ng total RNA was also used with QlAseq Targeted RNAscan Panels system from QIAGEN for comparison purpose. The same amount of 50 ng gDNA was also used with QlAseq Targeted DNA Panels system from QIAGEN for comparison purpose. The samples were then put on Illumina's MiSeq machine for sequencing.

Results: As shown in Table 1, compared to the standalone RNA library prep workflow (QIAseq Targeted RNAscan Panels system from QIAGEN), our method achieved around 24% of its enrichment efficiency on the 1^{st} strand cDNA, and around 40% of its enrichment efficiency on the 2^{nd} strand cDNA. Since RNAscan workflow had strand bias toward the 1^{st} strand, our method had less bias and improved strand balance. The effect of enrichment efficiency on RNA analysis deserves further exploration.

TABLE 1

| Workflow | RNAScan | Ours |
|---|---|---|
| Average UMIs/primer | 11061 | 2681 |
| 1^{st} strand | | |
| Average UMIs/primer | 5279 | 2077 |
| 2^{nd} strand | | |
| Ratio 2^{nd}/1^{st} | 0.48 | 0.77 |

UMI per SPE primer for RNA sample: Primers were divided into two groups based on the RNA strand they detected. As shown in Table 2, compared to the standalone DNA library prep workflow (QlAseq Targeted DNA Panels system from QIAGEN), our method achieved slightly better enrichment efficiency. Both of the methods had comparable sequencing specificity and uniformity.

TABLE 2

| Workflow | Targeted DNA Panels | Ours |
|---|---|---|
| Average UMIs/primer | 1471 | 1701 |
| Average reads/UMI | 3.4 | 3.0 |
| Overall specificity | 87% | 90% |
| (on-target reads/all reads) | | |
| Coverage uniformity | 24.9 | 21.6 |
| (T50) | | |

Sequencing specs for DNA sample in both methods: Sequence coverage uniformity was measured by T50, the percentage of total sequence throughput captured by the bottom 50% of a target region. In the perfect uniform scenario, the T50 value equals to 50.

Cross talk between DNA and RNA was also evaluated since they remained in the same reaction. Using the same 50 ng of DNA and RNA from THP-1 cell line, the effective leaking signal from RNA to DNA was only 0.75% of the real DNA signal, as measured by the total UMIs of the primers detecting both RNA and DNA. In this case, only the extremely highly expressed genes might have an effect on corresponding DNA copy number analysis. However, if DNA copy number analysis was limited on intron regions, this effect should disappear. The effective leaking signal from DNA to RNA was around 3% on average by the same measurement. Since there were only a few copies of genome DNA in each cell in most cases, this kind of leaking could only affect those extremely low expressing genes (less than 0.1 copy per cell), which might be lower than the background noise level. In conclusion, our method demonstrated minimal cross talk between DNA and RNA samples which might not have any significant effect in real cases.

The DNA library prepared by our method can be used for DNA variant detection and copy number analysis. The RNA library prepared by our method is suitable for gene expression analysis, fusion gene detection, and RNA variant detection. Multi-modal NGS panels can be developed based on our proposed method, and be used for biomarker screening, or targeted eQTL analysis.

Adapter for Ligation:

Equal molar mix and annealing of the following
2 oligos to make double strand adapter
(DNA ligation Adapter)

| | | |
|---|---|---|
| SEQ ID | /5Phos/GGACTCCAATNNNNNNNNACG | PAGE |
| NO: 2 | CTAAGAAAGATCGGAAGAGCACACGTCTG/3 | Purified |
| | ddC/ | |
| SEQ ID | ATT+GGAG+TCC*T/3Phos/ | STD |
| NO: 3 | | desalt |

Reverse Transcription Oligos:

| | | |
|---|---|---|
| SEQ ID NO: 4: | CGACTCACTATAGGGCTGGAATTCTGACGNN | PAGE |
| TSON10 T18NV | NNNNNNNNACGTTTTTTTTTTTTTTTTTTNV | Purified |
| oligo | | |
| SEQ ID NO: 5: | /5Me-isodC//iisodG//iisodG/TAAT | PAGE |
| TSON10 for TS | ACGACTCACTATAGGGCTGGAATTCTGACGN | Purified |
| oligo | NNNNNATCTGCrGrGrG | |

Target Enrichment Oligos:

| | | |
|---|---|---|
| SEQ ID NO: 6: | AGCAGTGGTATCAACGCAGAGTCAAGCAGAAG | STD |
| DNA boosting | ACGGCATACGAGATTCCGAAACGTGACTGGAG | desalt |
| primer | TTCAGACGTGTGCTCTTCCGATCTTTCTTAGCGT | |
| SEQ ID | GTGAGTGATGGTTGAGGATGTGTGCAAGCAGA | STD |
| NO: 7: RNA | AGACGGCATACGAGATTACGTACGGTGACTGG | desalt |
| boosting | AGTTCAGACGTGTGCTCTTCCGATCTCGACTCA | |
| primer | CTATAGGGCTGGAATTCT | |

For each primer, the first set of underlined nucleotides
is priming site for PCR amplification in Universal PCR
reactions, the second set of underlined nucleotides in the
middle is the sample idx (index) region, which can be
replaced with respective sample index sequences, and the
third set of underlined nucleotides is part of DNA or RNA
identifier used for PCR amplification in target enrichment
reactions.

uPCR Primers:

| | | |
|---|---|---|
| SEQ ID NO: 8: | AATGATACGGCGACCACCGAGATCTA | PAGE |
| IL2N5RS2 | CACTCTTTCCCTACACGACGCTCTTCC | Purified |
| Universal | GATCTNNNNNAATGTACAGTATTGCG | |
| primer | TTTTG | |
| SEQ ID NO: 9: | AAGCAGTGGTATCAACGCAGAGT | STD |
| DNA Universal | | desalt |
| Primer | | |
| SEQ ID NO: 10: | GTGAGTGATGGTTGAGGATGTGTG | STD |
| RNA Universal | | desalt |
| Primer | | |

SPE Primer Pool (Equal Molar Mix of the Following Oligos):

SEQ ID NO: 11:
AATGTACAGTATTGCGTTTTGAGCCCCAAGTCCTATGAGAACCTCTG
SEQ ID NO: 12:
AATGTACAGTATTGCGTTTTGTGGCACCAGCGATCAGGTCCTTTAT
SEQ ID NO: 13:
AATGTACAGTATTGCGTTTTGCTGAGTGGAGTCACAGCGGAGATAGT
SEQ ID NO: 14:
AATGTACAGTATTGCGTTTTGTGTTCCACCAGTAACAACAGTTGAATGTCC
SEQ ID NO: 15:
AATGTACAGTATTGCGTTTTGGTGTGAGGAACATACTAGTGCTTTGCAAGT
SEQ ID NO: 16:
AATGTACAGTATTGCGTTTTGTTCAAAGTTGGGTCTGCTTCAGTCCAAAG
SEQ ID NO: 17:
AATGTACAGTATTGCGTTTTGCCCCCAGCTTCTTCTCTCTGCACTAAG
SEQ ID NO: 18:
AATGTACAGTATTGCGTTTTGGCCTTCCCAACATGCATTCTAACTTCTTCC
SEQ ID NO: 19:
AATGTACAGTATTGCGTTTTGCCAGCTACTCTCAAAATCAGCATCCTTTGG
SEQ ID NO: 20:
AATGTACAGTATTGCGTTTTGCCAGTCCTTCTGTGAGTCTATCCTCAGTTC
SEQ ID NO: 21:
AATGTACAGTATTGCGTTTTGAGAGCGAACCAAGAATGCCTGTTTACAG
SEQ ID NO: 22:
AATGTACAGTATTGCGTTTTGGAGAGGCACGAGAACACACATCTATTCTG
SEQ ID NO: 23:
AATGTACAGTATTGCGTTTTGTTCTCTTCAGAAGTTCCTTCGTCATCCTT
SEQ ID NO: 24:
AATGTACAGTATTGCGTTTTGTGATGACATGCCCCATCACTAAAACAC
SEQ ID NO: 25:
AATGTACAGTATTGCGTTTTGTGATAGAGACATGATGTAACCGTGGGAATTTCTTC
SEQ ID NO: 26:
AATGTACAGTATTGCGTTTTGCGTTCTAAGAGAGTGACAGAAAGGTAAAGAGGAG
SEQ ID NO: 27:
AATGTACAGTATTGCGTTTTGATCACAAAGTATCTTTTTCTGTGGCTTAGAAATCTT
SEQ ID NO: 28:
AATGTACAGTATTGCGTTTTGTCAAATGTTAGCTCATTTTTGTTAATGGTGGCTTTT
SEQ ID NO: 29:
AATGTACAGTATTGCGTTTTGTGTCACATTATAAAGATTCAGGCAATGTTTGTTAGT
SEQ ID NO: 30:
AATGTACAGTATTGCGTTTTGAGTTTGTATGCAACATTTCTAAAGTTACCTACTTGT
SEQ ID NO: 31:
AATGTACAGTATTGCGTTTTGAAAATCTGTTTTCCAATAAATTCTCAGATCCAGGAA
SEQ ID NO: 32:
AATGTACAGTATTGCGTTTTGCGACCCAGTTACCATAGCAATTTAGTGAAATAACTA
SEQ ID NO: 33:
AATGTACAGTATTGCGTTTTGAGAGGCGCTATGTGTATTATTATAGCTACCTGTTAA
SEQ ID NO: 34:
AATGTACAGTATTGCGTTTTGCGTTTTTGACAGTTTGACAGTTAAAGGCATTTCC
SEQ ID NO: 35:
AATGTACAGTATTGCGTTTTGCTGTCCTTATTTTGGATATTTCTCCCAATGAAAGTA
SEQ ID NO: 36:
AATGTACAGTATTGCGTTTTGGACTTTTTGCAAATGTTTAACATAGGTGACAGATTT
SEQ ID NO: 37:
AATGTACAGTATTGCGTTTTGAAGTAGAAAATGGAAGTCTATGTGATCAAGAAATCG
SEQ ID NO: 38:
AATGTACAGTATTGCGTTTTGGGCCTCTTAAAGATCATGTTTGTTACAGTGCTTA
SEQ ID NO: 39:
AATGTACAGTATTGCGTTTTGACAAGATTGGTCAGGAAAAGAGAATTGTTCCTATAA
SEQ ID NO: 40:
AATGTACAGTATTGCGTTTTGAGACCCTGTCTCAAAAGTAAAAAGTAAGTTAACATG
SEQ ID NO: 41:
AATGTACAGTATTGCGTTTTGTCAGTGTCTTCCAAATCCTTATGTATAGCAGCAAT
SEQ ID NO: 42:
AATGTACAGTATTGCGTTTTGAGGGTCGAGGAAGCCAGTTTACATCAA
SEQ ID NO: 43:
AATGTACAGTATTGCGTTTTGAACAAAAAGATATTTTCAATATTTCTGCGCAGGTTT
SEQ ID NO: 44:
AATGTACAGTATTGCGTTTTGGTCTCGACTTGAATTGCAAAAAGATGTTAGAAAAGC
SEQ ID NO: 45:
AATGTACAGTATTGCGTTTTGAAAATGTTGGCAGTCATAACATTTGAAACTAATGGA
SEQ ID NO: 46:
AATGTACAGTATTGCGTTTTGAGCCTCAAACAGGTTGGTTTTAAATTTGAAGTCT
SEQ ID NO: 47:
AATGTACAGTATTGCGTTTTGCCTCTGTGTGTATGTTTTAACTACAAAGCGAAACA
SEQ ID NO: 48:
AATGTACAGTATTGCGTTTTGGATTCACCTGGTAATGAGGAAAACAGCTTTAAAATC
SEQ ID NO: 49:
AATGTACAGTATTGCGTTTTGAGATCTGCTGAAAAGAAATTTGTTAAAGCACAATT
SEQ ID NO: 50:
AATGTACAGTATTGCGTTTTGCGGCATCCCCTACATCGAGACCTC
SEQ ID NO: 51:
AATGTACAGTATTGCGTTTTGCAGGGAGCAGATCAAACGGGTGAAG
SEQ ID NO: 52:
AATGTACAGTATTGCGTTTTGCAAGTCTTTTGAGGACATCCACCAGTACAG
SEQ ID NO: 53:
AATGTACAGTATTGCGTTTTGACGTGCCTGTTGGACATCCTGGATA
SEQ ID NO: 54:
AATGTACAGTATTGCGTTTTGCCTGTACTGGTGGATGTCCTCAAAAGACT
SEQ ID NO: 55:
AATGTACAGTATTGCGTTTTGCCCTGAGGAGCGATGACGGAATATAAGC
SEQ ID NO: 56:
AATGTACAGTATTGCGTTTTGGTCGTATTCGTCCACAAAATGGTTCTGGATC
SEQ ID NO: 57:
AATGTACAGTATTGCGTTTTGTGACTGGCAATTGTGTCAACAGGTGAAAA
SEQ ID NO: 58:
AATGTACAGTATTGCGTTTTGCGCCAGCTGGAGTTTGGTCATGTTT
SEQ ID NO: 59:
AATGTACAGTATTGCGTTTTGAATCCCTCTCATCACAATTTCATTCCACAATAGTTT
SEQ ID NO: 60:
AATGTACAGTATTGCGTTTTGTCAACAACAAAGAGAATCATGAAATCAACCCTAGC
SEQ ID NO: 61:
AATGTACAGTATTGCGTTTTGGATATGGAGCCAGCGTGTTCCGATT
SEQ ID NO: 62:
AATGTACAGTATTGCGTTTTGGGCGCGGAAAGTCCTCACTCTC
SEQ ID NO: 63:
AATGTACAGTATTGCGTTTTGTATGGTGAGGTTCGGCGTGTTTAAACG
SEQ ID NO: 64:
AATGTACAGTATTGCGTTTTGTGGTGACAAAGTTAGAAGGGTCCATGG
SEQ ID NO: 65:
AATGTACAGTATTGCGTTTTGCTTCTTTACCACCCCAGATACGACGACTA
SEQ ID NO: 66:
AATGTACAGTATTGCGTTTTGCGCTCGTGGTGGTAGTCGTCGTAT
SEQ ID NO: 67:
AATGTACAGTATTGCGTTTTGCCAGGAGGCCCTTTCTGTTTACAACC
SEQ ID NO: 68:
AATGTACAGTATTGCGTTTTGCCCACAAGCCCAAAATATTCTACTCACTTTGC
SEQ ID NO: 69:
AATGTACAGTATTGCGTTTTGATCGCCTGCATCAAGGAAAAGGTAATGG
SEQ ID NO: 70:
AATGTACAGTATTGCGTTTTGCGCGTAAGGATAGCAACTGAGGTTATCAC
SEQ ID NO: 71:
AATGTACAGTATTGCGTTTTGCGACCTGACGTAACCCCTTGCTTATC
SEQ ID NO: 72:
AATGTACAGTATTGCGTTTTGGGAAATGCTCTCACGTAGTCTCTCATGTCT
SEQ ID NO: 73:
AATGTACAGTATTGCGTTTTGGTCATAACCCGAAGAACAATGTTGCCACTA
SEQ ID NO: 74:
AATGTACAGTATTGCGTTTTGGTCAGCTCAGGATAAAGCACGGATGGATA
SEQ ID NO: 75:
AATGTACAGTATTGCGTTTTGCTCAGGATAAAAGCTTCCTTCTTAACAAGTTTTTCC
SEQ ID NO: 76:
AATGTACAGTATTGCGTTTTGAGAGATTGTTCCCTTGCATTGACCTCTTTTTC
SEQ ID NO: 77:
AATGTACAGTATTGCGTTTTGCCCCTCACCTTTGGAATTTACAGTCTGAA
SEQ ID NO: 78:
AATGTACAGTATTGCGTTTTGTAGGTTCTTCAGGTCTCTACACTCTCCTTTAAACT
SEQ ID NO: 79:
AATGTACAGTATTGCGTTTTGGAGAAGGAGTGCAATGCCAAGATTATGATCC
SEQ ID NO: 80:
AATGTACAGTATTGCGTTTTGGACGTTCTCCATTGTATTGGCAGTAACCA
SEQ ID NO: 81:
AATGTACAGTATTGCGTTTTGCACATCTCACAGGCTCTAAAGGAATTCTATATCCTA
SEQ ID NO: 82:
AATGTACAGTATTGCGTTTTGGAGGCAAGAGGTGAGTAGTACCAATACTGTC
SEQ ID NO: 83:
AATGTACAGTATTGCGTTTTGGAGCCCCTCCGCTTACTTGTAATCTG
SEQ ID NO: 84:
AATGTACAGTATTGCGTTTTGCCAGTAAAACGTATTGAGAAAAAGGTAAAAGCGTTA
SEQ ID NO: 85:
AATGTACAGTATTGCGTTTTGGCTCAGAATAAATCGTAACAATCTCAAAGTGCATTT
SEQ ID NO: 86:
AATGTACAGTATTGCGTTTTGTGAGGTGTCCACAGGGCTCAATCTTTAC
SEQ ID NO: 87:
AATGTACAGTATTGCGTTTTGCCCCTTGTATCAGTAAAGGCTATATAATACCGAATT
SEQ ID NO: 88:
AATGTACAGTATTGCGTTTTGTCATGAAGAGAGTATCATCAGCTCGTTCATCATC
SEQ ID NO: 89:
AATGTACAGTATTGCGTTTTGTGTCCTTTCTGCCGATGTGAAATTAAAGGTAC
SEQ ID NO: 90:
AATGTACAGTATTGCGTTTTGTCGCCCCAAATAATTTCCTGCGAACA
SEQ ID NO: 91:
AATGTACAGTATTGCGTTTTGCTCATACCTCCATTCCAAGCTTTCATTGTCTC
SEQ ID NO: 92:
AATGTACAGTATTGCGTTTTGCCTGCCCTTATTTTTAACAGCAGGAACGAAT
SEQ ID NO: 93:
AATGTACAGTATTGCGTTTTGTCGATAGCGAAAGTCCTCTTTGGTCAG
SEQ ID NO: 94:
AATGTACAGTATTGCGTTTTGGTTAAAGACCAACCACTAACTAAGAGACTTTCCAAG
SEQ ID NO: 95:
AATGTACAGTATTGCGTTTTGAAACCTCTTCCAGTACCTTCTTCATGGTTCT
SEQ ID NO: 96:
AATGTACAGTATTGCGTTTTGTTTCCAGGTGATGTGCTCTATGAACTCCTT
SEQ ID NO: 97:
AATGTACAGTATTGCGTTTTGGGAGCGGTGCAACAGTTCAATGGT
SEQ ID NO: 98:
AATGTACAGTATTGCGTTTTGCATCCGTGGATAATGTGCACCATAACC
SEQ ID NO: 99:
AATGTACAGTATTGCGTTTTGTCGGAGAGCCTGGACTGTTTGAAATC
SEQ ID NO: 100:
AATGTACAGTATTGCGTTTTGAAGCCAGGTCTTCCCGATGAGAGAG
SEQ ID NO: 101:
AATGTACAGTATTGCGTTTTGGGCACTCCGTGGATTTCAAACAGTC
SEQ ID NO: 102:
AATGTACAGTATTGCGTTTTGCAGATATCTGCTGCCCTTTTACCTTATGGTTT
SEQ ID NO: 103:
AATGTACAGTATTGCGTTTTGTGTAGACTGCTTTGGGATTACGTCTATCAGTTG
SEQ ID NO: 104:
AATGTACAGTATTGCGTTTTGGGAAAGGAGAAAAAGGAAGTGCTACCTGAAC
SEQ ID NO: 105:
AATGTACAGTATTGCGTTTTGTTTTTCTCCCTTCCTCCTTTGAACAAACAG
SEQ ID NO: 106:
AATGTACAGTATTGCGTTTTGACAGCTTTAGGAAAATGGAATCTCTTACCTCCTC
SEQ ID NO: 107:
AATGTACAGTATTGCGTTTTGGGGTGTTATGGTCGCGTTGGATTTCTG
SEQ ID NO: 108:
AATGTACAGTATTGCGTTTTGGCTACGGCGTGCAACTCACAGAAC
SEQ ID NO: 109:
AATGTACAGTATTGCGTTTTGACCGACCTCTTCCAGCGCTACTT
SEQ ID NO: 110:
AATGTACAGTATTGCGTTTTGCGGGCAGGGCTTACTTACCTTGG
SEQ ID NO: 111:
AATGTACAGTATTGCGTTTTGTAGCTACTGCCTGCCTTCGAAGAACGAT
SEQ ID NO: 112:
AATGTACAGTATTGCGTTTTGTGTGGGTGGAAAAAGATGTGGTTAAGAAACAAC
SEQ ID NO: 113:
AATGTACAGTATTGCGTTTTGCCCCCATATAGCTTAATCTGATGGGCATC
SEQ ID NO: 114:
AATGTACAGTATTGCGTTTTGGAAAGAGCATCAGGAACAAGCCTTGAGTAC
SEQ ID NO: 115:
AATGTACAGTATTGCGTTTTGTTGAGATGCCTGACAACCTTTACACCTTTG
SEQ ID NO: 116:
AATGTACAGTATTGCGTTTTGCTCTAGGGCTGAGGGAATATGCATCTCT
SEQ ID NO: 117:
AATGTACAGTATTGCGTTTTGCGTACCCAGAAGACAATGGCCTAGCTAT
SEQ ID NO: 118:
AATGTACAGTATTGCGTTTTGGGGCAGCACAGATTCCCTTAACCA
SEQ ID NO: 119:
AATGTACAGTATTGCGTTTTGCCATACCTTGGCTATCCCCTGAAAGTTG
SEQ ID NO: 120:
AATGTACAGTATTGCGTTTTGGCCCTGATGCTCATGGAGTGTTCCT
SEQ ID NO: 121:
AATGTACAGTATTGCGTTTTGCCTGGTGGTTGGGAGACGACTAC
SEQ ID NO: 122:
AATGTACAGTATTGCGTTTTGTGCTGACAGGACACAGAACAAGATACCT
SEQ ID NO: 123:
AATGTACAGTATTGCGTTTTGGGTACAGGTATCTTGTTCTGTGTCCTGTCAG
SEQ ID NO: 124:
AATGTACAGTATTGCGTTTTGGAGTCCCGGGCTCGATTCACAG
SEQ ID NO: 125:
AATGTACAGTATTGCGTTTTGCTGGTCAGAGAGGTGTGTACTGATTGTCT
SEQ ID NO: 126:
AATGTACAGTATTGCGTTTTGAGGAAAGATCAATTACATTCACAAGTTCACACTTCT
SEQ ID NO: 127:
AATGTACAGTATTGCGTTTTGCTGCACAGTTCAGAGGATATTTAAGCTCAATGAC
SEQ ID NO: 128:
AATGTACAGTATTGCGTTTTGCACAGACCGTCATGCATTTCTGACACTC
SEQ ID NO: 129:
AATGTACAGTATTGCGTTTTGAGGCTGGTACCTGCTCTTCTTCAATC
SEQ ID NO: 130:
AATGTACAGTATTGCGTTTTGCGAAATCAAACAGTTGTCTATCAGAGCCTGTC
SEQ ID NO: 131:
AATGTACAGTATTGCGTTTTGACAAAAGAAAAGAAGTCATGTCTGTATGTGGAAA
SEQ ID NO: 132:
AATGTACAGTATTGCGTTTTGTCCAGGATAATACACATCACAGTAAATAACACTCTG
SEQ ID NO: 133:
AATGTACAGTATTGCGTTTTGCATCCTCTTTGTCATCAAGCTACAGTCTTTTTGA
SEQ ID NO: 134:
AATGTACAGTATTGCGTTTTGCTCCCATTTTTGTGCATCTTTGTTGCTGTC
SEQ ID NO: 135:
AATGTACAGTATTGCGTTTTGCAGAACTGCCTATTCCTAACTGACTCATCATTTC
SEQ ID NO: 136:
AATGTACAGTATTGCGTTTTGGAATTCTGTTTCATCGCTGAGTGACACTCTTTT
SEQ ID NO: 137:
AATGTACAGTATTGCGTTTTGTTTTTACCTTTGCTTTTACCTTTTTGTACTTGTGAC
SEQ ID NO: 138:
AATGTACAGTATTGCGTTTTGAGAAGGAGTCTGGAATAGAAAGGCTAACAGAA
SEQ ID NO: 139:
AATGTACAGTATTGCGTTTTGCACAAGATGTGCCAAGGGAATTGTATGC
SEQ ID NO: 140:
AATGTACAGTATTGCGTTTTGAAGAGTCAATAGGTCAGAGAGTTTTATGTTCTTCCA
SEQ ID NO: 141:
AATGTACAGTATTGCGTTTTGACTGATCTTCTCAAAGTCGTCATCCTTCAGT
SEQ ID NO: 142:
AATGTACAGTATTGCGTTTTGACCCTGAGAAATAATCCAATTACCTGTTAATCAAGG
SEQ ID NO: 143:
AATGTACAGTATTGCGTTTTGAAAAGGTATTGAGTAAAATCAGTCTTCCTTCTACCC
SEQ ID NO: 144:
AATGTACAGTATTGCGTTTTGCCTTCCTCCCTCTTTCTTTCATAAAACCTCTCTT
SEQ ID NO: 145:
AATGTACAGTATTGCGTTTTGGCCAGAGCCACCCAACTCTTAAGG
SEQ ID NO: 146:
AATGTACAGTATTGCGTTTTGTGGAAGAGGAATTTAATAACGAACGTTTTAAGAGGA
SEQ ID NO: 147:
AATGTACAGTATTGCGTTTTGGCATCTACTGCCGAGGATGTTCCAAG
SEQ ID NO: 148:
AATGTACAGTATTGCGTTTTGCACAGTGAGCTCAAGTGCGACATCA
SEQ ID NO: 149:
AATGTACAGTATTGCGTTTTGCCGACTGGCCATCTCCTCGTAG
SEQ ID NO: 150:
AATGTACAGTATTGCGTTTTGGTACCAGCGCGACTACGAGGAGAT
SEQ ID NO: 151:
AATGTACAGTATTGCGTTTTGTCTTTTCTGTCAAATGGAGATGATCTCTTCTGACTC
SEQ ID NO: 152:
AATGTACAGTATTGCGTTTTGGGGAGCCCATCATCTGCAAAAACATCC
SEQ ID NO: 153:
AATGTACAGTATTGCGTTTTGAAGCTGAAGAAGATGTGGAAAAGTCCCAATG
SEQ ID NO: 154:
AATGTACAGTATTGCGTTTTGGCGTGGGATGTTTTTGCAGATGATGG
SEQ ID NO: 155:
AATGTACAGTATTGCGTTTTGCGACGCTGAGGACGCTATGGATG
SEQ ID NO: 156:
AATGTACAGTATTGCGTTTTGGCTGAGGCGCGTCTTCGAGAAG
SEQ ID NO: 157:
AATGTACAGTATTGCGTTTTGGCGCTTGTCGTGAAAGCGAACGA
SEQ ID NO: 158:
AATGTACAGTATTGCGTTTTGGCTGCCCGCCCAGTTGTTACT
SEQ ID NO: 159:
AATGTACAGTATTGCGTTTTGAGACTCTGGACTGATGAAGCAATTCTGAGT
SEQ ID NO: 160:
AATGTACAGTATTGCGTTTTGTCACCGGTGACACCTTAAAACCAAAGC
SEQ ID NO: 161:
AATGTACAGTATTGCGTTTTGGGCTCCTTTGTACCTCCTCCATCTTGATC
SEQ ID NO: 162:
AATGTACAGTATTGCGTTTTGGTCAGTTGTCTAACAATAACAAAGATCTGCTCTTGG
SEQ ID NO: 163:
AATGTACAGTATTGCGTTTTGGGTGGGCAGCAAGAAAAAGTCCAGTAAA
SEQ ID NO: 164:
AATGTACAGTATTGCGTTTTGGCCAAGGCTTTCTCTGGCATGATCTTTT
SEQ ID NO: 165:
AATGTACAGTATTGCGTTTTGGGATAACTTTCTCAGCATTTCCACCAGTTTCAAG
SEQ ID NO: 166:
AATGTACAGTATTGCGTTTTGTGTCCCTAAGTTGAGTAAAATGATAGAGAATGAGTC
SEQ ID NO: 167:
AATGTACAGTATTGCGTTTTGGCTGCCAGAAATCCAGCATCCAAAATTTG
SEQ ID NO: 168:
AATGTACAGTATTGCGTTTTGGTCGCTTTCTTTTCTTAGTGCCAGGAAACT
SEQ ID NO: 169:
AATGTACAGTATTGCGTTTTGACAGTCGAGACGATTCATGAGGGAACTTC
SEQ ID NO: 170:
AATGTACAGTATTGCGTTTTGGGAAAGCTCGGCGTGTTGGATAAGAAG
SEQ ID NO: 171:
AATGTACAGTATTGCGTTTTGACGCCACAAGTGACTGAAAGTTGGAAG
SEQ ID NO: 172:
AATGTACAGTATTGCGTTTTGTGATGGGCTGGAGATTTGGCATAGTTTTC
SEQ ID NO: 173:
AATGTACAGTATTGCGTTTTGCTATGCACCCACTTTCAACACAGTTAGGT
SEQ ID NO: 174:
AATGTACAGTATTGCGTTTTGGCTTGGTCAGAAGTGCTGTTGTTGTC
SEQ ID NO: 175:
AATGTACAGTATTGCGTTTTGCGTGGGCCAGAAAGTTGTCCACAATG
SEQ ID NO: 176:
AATGTACAGTATTGCGTTTTGGGGATATGGATTCTCGTGGTAGAAGGTGTAA
SEQ ID NO: 177:
AATGTACAGTATTGCGTTTTGCTAATCACCAAGTTCCAAGTGTTCAGAATCTCC
SEQ ID NO: 178:
AATGTACAGTATTGCGTTTTGACCGTAATAACCAAGGTTCATCATAGGCATTGAT
SEQ ID NO: 179:
AATGTACAGTATTGCGTTTTGTCCCAGTGGAAGTTACTATGCACCCTAT
SEQ ID NO: 180:
AATGTACAGTATTGCGTTTTGTGCTTATGCTTGTGTTTGTGTTTCCTCTTATGG
SEQ ID NO: 181:
AATGTACAGTATTGCGTTTTGGCTTCTGTTTCTCCTTATGCTTGTTCTTCTCAC
SEQ ID NO: 182:
AATGTACAGTATTGCGTTTTGCCTGAGTGGTCTTTTTGCAGGCAAAG
SEQ ID NO: 183:
AATGTACAGTATTGCGTTTTGCCGGCCACAAAGCTTCTAAGAACAAC
SEQ ID NO: 184:
AATGTACAGTATTGCGTTTTGGCGGTTCATCTTGAAGGCTTGGATGT
SEQ ID NO: 185:
AATGTACAGTATTGCGTTTTGTTCAGTGAAATGAACCCTTCGAATGACAAG
SEQ ID NO: 186:
AATGTACAGTATTGCGTTTTGCTCCTCCTCCTCTTTGCGTTTCTTGTC
SEQ ID NO: 187:
AATGTACAGTATTGCGTTTTGGCAGCAGAGAAACAAATGAAGGACAAACAG
SEQ ID NO: 188:
AATGTACAGTATTGCGTTTTGTAAGGAGGAGGAAGAAGACAAGAAACGCAAA
SEQ ID NO: 189:
AATGTACAGTATTGCGTTTTGTAAGGCAGGTCTGTGAGCACAAAATTTGG
SEQ ID NO: 190:
AATGTACAGTATTGCGTTTTGTGGAGCTGACCAGTGACAATGACC
SEQ ID NO: 191:
AATGTACAGTATTGCGTTTTGGGCCAAGAAGTCGGTGGACAAGAAC
SEQ ID NO: 192:
AATGTACAGTATTGCGTTTTGGCGCAGGCGGTCATTGTCACTG
SEQ ID NO: 193:
AATGTACAGTATTGCGTTTTGTTGCTGTTCTTGTCCACCGACTTCTTG
SEQ ID NO: 194:
AATGTACAGTATTGCGTTTTGGCAGTGCGCGATCTGGAACTG
SEQ ID NO: 195:
AATGTACAGTATTGCGTTTTGCGGCGGCGACTTTGACTACCC
SEQ ID NO: 196:
AATGTACAGTATTGCGTTTTGGAGCACGAGACGTCCATCGACATC
SEQ ID NO: 197:
AATGTACAGTATTGCGTTTTGCGGCCAGGAACTCGTCGTTGAA
SEQ ID NO: 198:
AATGTACAGTATTGCGTTTTGGCCATGCCGGGAGAACTCTAACTC
SEQ ID NO: 199:
AATGTACAGTATTGCGTTTTGTGTAACCCTCCTAAGTGTTCATACGTTGTCTTG
SEQ ID NO: 200:
AATGTACAGTATTGCGTTTTGGTCTTGGTCTCTGTTATATCTTGAGTCTAGAACAGT
SEQ ID NO: 201:
AATGTACAGTATTGCGTTTTGCAGGAGAACATGGAGGCGAGAAGAAAAT
SEQ ID NO: 202:
AATGTACAGTATTGCGTTTTGGGGAAAGATTGGATGCCGGGAATCAAC
SEQ ID NO: 203:
AATGTACAGTATTGCGTTTTGCGGAGGCTTGATTAGGTAGGAGGTG
SEQ ID NO: 204:
AATGTACAGTATTGCGTTTTGGCGGCAGCTCAACGAGAATAAACA
SEQ ID NO: 205:
AATGTACAGTATTGCGTTTTGGCCCGCATCCTTACTCCGCTTATC
SEQ ID NO: 206:
AATGTACAGTATTGCGTTTTGGCTGGTTTCAAGGTAAGTGGACTCTTCC
SEQ ID NO: 207:
AATGTACAGTATTGCGTTTTGGGGAATGACTGACGGAGAATCCCAAC
SEQ ID NO: 208:
AATGTACAGTATTGCGTTTTGCTAAGACCGAGAGCCTGTAGGAGCTTT
SEQ ID NO: 209:
AATGTACAGTATTGCGTTTTGGCCGGGCTTGTCTGGTCATCT
SEQ ID NO: 210:
AATGTACAGTATTGCGTTTTGCAGCTCACCTCCAAAAAGGCAAAATTCTTG
SEQ ID NO: 211:
AATGTACAGTATTGCGTTTTGGCAGGAGGCCATGATGGATTTCTTCAA
SEQ ID NO: 212:
AATGTACAGTATTGCGTTTTGCATGAGTGAAAGGAAAGAGGAAATCCCAATCC
SEQ ID NO: 213:
AATGTACAGTATTGCGTTTTGCCTATCTTCCACAGTACTTACACAACTTCCTAAGC
SEQ ID NO: 214:
AATGTACAGTATTGCGTTTTGCTCGCCGTAGACTGTCCAGGTTTT
SEQ ID NO: 215:
AATGTACAGTATTGCGTTTTGCTCACCTGATCCGTGACGTTGATGTC
SEQ ID NO: 216:
AATGTACAGTATTGCGTTTTGGCCCTGATGGACTCTCGGCTACT
SEQ ID NO: 217:
AATGTACAGTATTGCGTTTTGGAGAAAGATCAGGAACACTTGTCCCCTACTAG
SEQ ID NO: 218:
AATGTACAGTATTGCGTTTTGGTCCTCCACGATCTCCTCATACTCCTC
SEQ ID NO: 219:
AATGTACAGTATTGCGTTTTGTCGATGGACTTGACAAGCCCGTACTT
SEQ ID NO: 220:
AATGTACAGTATTGCGTTTTGCTGGACGACGAGGAGTATGAGGAGATC
SEQ ID NO: 221:
AATGTACAGTATTGCGTTTTGTACCAGAAGTCCCGGCGGTGATAAG
SEQ ID NO: 222:
AATGTACAGTATTGCGTTTTGGTTCACCTCTGTGTTTGACTGCCAGAAA
SEQ ID NO: 223:
AATGTACAGTATTGCGTTTTGCAATGAGTATTCTCTTCATTTCAGGTCAGTTGATTT
SEQ ID NO: 224:
AATGTACAGTATTGCGTTTTGGGCTGCTTTCTTGAAGGCTATTGGGTAT
SEQ ID NO: 225:
AATGTACAGTATTGCGTTTTGAGGAGACTGGAATTCTCGAATAAGGATTAACA
SEQ ID NO: 226:
AATGTACAGTATTGCGTTTTGGCATAGTTAAAACCTGTGTTTGGTTTTGTAGGTCTT
SEQ ID NO: 227:
AATGTACAGTATTGCGTTTTGCTCTGTGTTGGCGGATACCCTTCCATA
SEQ ID NO: 228:
AATGTACAGTATTGCGTTTTGGGCATTCCTTCTTTATTGCCCTTCTTAAAAGC
SEQ ID NO: 229:
AATGTACAGTATTGCGTTTTGGCTGCTGGTCTGGCTACTATGATCTCTAC
SEQ ID NO: 230:
AATGTACAGTATTGCGTTTTGGCACACAGCTTTTAAGAAGGGCAATAAAGAAG
SEQ ID NO: 231:
AATGTACAGTATTGCGTTTTGTGTATGTTTAATTCTGTACATGAGCATTTCATCAGT
SEQ ID NO: 232:
AATGTACAGTATTGCGTTTTGATTTCATACCTTGCTTAATGGGTGTAGATACCAAAA
SEQ ID NO: 233:
AATGTACAGTATTGCGTTTTGTTGGCGTCAAATGTGCCACTATCACTC
SEQ ID NO: 234:
AATGTACAGTATTGCGTTTTGTTCTCTTTCAAGCTATGATTTAGGCATAGAGAATCG
SEQ ID NO: 235:
AATGTACAGTATTGCGTTTTGCTGCAGTTGTAGGTTATAACTATCCATTTGTCTGAA
SEQ ID NO: 236:
AATGTACAGTATTGCGTTTTGCCCTAGGTCAGATCACCCAGTCAGTTAAAAC
SEQ ID NO: 237:
AATGTACAGTATTGCGTTTTGTGGTTAAAGGTCAGCCCACTTACCAGATATG
SEQ ID NO: 238:
AATGTACAGTATTGCGTTTTGGGGTATGCTCCCCATTTAGAGGATAAGG
SEQ ID NO: 239:
AATGTACAGTATTGCGTTTTGACGTCAGATCTACAGCGAACACAACTACT
SEQ ID NO: 240:
AATGTACAGTATTGCGTTTTGAGTGGTGCCAGACTCACATTCAGTTCTAA
SEQ ID NO: 241:
AATGTACAGTATTGCGTTTTGCTTGGCCAGTTCCTTTCTCTAATGTATCATCTC
SEQ ID NO: 242:
AATGTACAGTATTGCGTTTTGAAGTTTTCTTGTCTAGTATCACTTTCCCTCATAGG
SEQ ID NO: 243:
AATGTACAGTATTGCGTTTTGGGGCTCAACAGATGGTATGTGTTCTCTG
SEQ ID NO: 244:
AATGTACAGTATTGCGTTTTGGCTCTCGTTTCTAACAGTTCTTTGCATTGGATA
SEQ ID NO: 245:
AATGTACAGTATTGCGTTTTGGAGGTGACCTTCAAAGTCAGAGGCTGTAT
SEQ ID NO: 246:
AATGTACAGTATTGCGTTTTGGAGCAACCATCCCATCTGTCCTTGTAAC
SEQ ID NO: 247:
AATGTACAGTATTGCGTTTTGGGACAAGGATGAGAAACCCAATTGGAACC
SEQ ID NO: 248:
AATGTACAGTATTGCGTTTTGCGGTCCGCCAAAAGATCCCAGATTC
SEQ ID NO: 249:
AATGTACAGTATTGCGTTTTGGGAGGCCACTAACCCACTTGTGATG
SEQ ID NO: 250:
AATGTACAGTATTGCGTTTTGTCCAGTTTCCTAGAGGATGTAATGGGATTTGTC
SEQ ID NO: 251:
AATGTACAGTATTGCGTTTTGTCACATTTGGAGATGAGAAACGAGGTGTTCT
SEQ ID NO: 252:
AATGTACAGTATTGCGTTTTGCCCTTGGCCTGTAACATTGCTCTGATC
SEQ ID NO: 253:
AATGTACAGTATTGCGTTTTGCACCTCGTTTCTCATCTCCAAATGTGATCTC
SEQ ID NO: 254:
AATGTACAGTATTGCGTTTTGCCAGTAGCTTTCCTGTTCTCGGCATT
SEQ ID NO: 255:
AATGTACAGTATTGCGTTTTGGCAGCGTCAAGAATGAGAAGACTTTTGTG
SEQ ID NO: 256:
AATGTACAGTATTGCGTTTTGTTGCCCTTCTGGAAATTACCCCGAGA
SEQ ID NO: 257:
AATGTACAGTATTGCGTTTTGAGTTCCACCAGCTTTAATTATTCCTCTAGCTCTC
SEQ ID NO: 258:
AATGTACAGTATTGCGTTTTGGTTTCCCATGGCCATAATTTATTATCTCACCACAA
SEQ ID NO: 259:
AATGTACAGTATTGCGTTTTGGTCACGATGACTGTATTGGACCCTCAA
SEQ ID NO: 260:
AATGTACAGTATTGCGTTTTGTCCAGACCTTTGCTTTAGATTGGCAATTATTACTG
SEQ ID NO: 261:
AATGTACAGTATTGCGTTTTGCCCTAACAACACAGAAGCAAAGCGTTCTTT
SEQ ID NO: 262:
AATGTACAGTATTGCGTTTTGCGCCCTCCTACCACCTGTACTACG
SEQ ID NO: 263:
AATGTACAGTATTGCGTTTTGACTATCCAGGCGCCTTCACCTACTC
SEQ ID NO: 264:
AATGTACAGTATTGCGTTTTGCTCCTAGGCGGTATCATCCTGGGTAG
SEQ ID NO: 265:
AATGTACAGTATTGCGTTTTGTCTGATTCTCTTCAGATACAAGGCAGATCC
SEQ ID NO: 266:
AATGTACAGTATTGCGTTTTGGCAGATACTTGGACTTGAGTAGGCTTATTAAACC
SEQ ID NO: 267:
AATGTACAGTATTGCGTTTTGGCGGCTCTATAAAGAATTGTCCTTATTTTCGAACTT
SEQ ID NO: 268:
AATGTACAGTATTGCGTTTTGGTTCGAGGCCTTTCTCTGAGCATCAAG
SEQ ID NO: 269:
AATGTACAGTATTGCGTTTTGACATCGGCAGAAACTAGATGATCAGACCAA
SEQ ID NO: 270:
AATGTACAGTATTGCGTTTTGTTTAGGAAATCCACAATACTTTTTCTGATCTCTTCC
SEQ ID NO: 271:
AATGTACAGTATTGCGTTTTGGCCACCAACCTCATTCTGTTTTGTTCTCTATC
SEQ ID NO: 272:
AATGTACAGTATTGCGTTTTGCTGCATTTGTCCTTTGACTGGTGTTTAGGT
SEQ ID NO: 273:
AATGTACAGTATTGCGTTTTGCTTCGACCGACAAACCTGAGGTCATTAAATC
SEQ ID NO: 274:
AATGTACAGTATTGCGTTTTGCCCCACATCCCAAGCTAGGAAGACC
SEQ ID NO: 275:
AATGTACAGTATTGCGTTTTGCGGGCCAGTACCTTGAAAGCGATG
SEQ ID NO: 276:
AATGTACAGTATTGCGTTTTGCTAACTCAATCGGCTTGTTGTGATGCGTAT
SEQ ID NO: 277:
AATGTACAGTATTGCGTTTTGCCCTCCTGGACTGTTAGTAACTTAGTCTCC
SEQ ID NO: 278:
AATGTACAGTATTGCGTTTTGCCCTCCGAGCTCCGCGAAAAT
SEQ ID NO: 279:
AATGTACAGTATTGCGTTTTGGTGCTAAAAAGTGTAAGAAGAAATGAGCTAGCAAAA
SEQ ID NO: 280:
AATGTACAGTATTGCGTTTTGCATATGCCTCAGTTTGAATTCCTCTCACAAACAA
SEQ ID NO: 281:
AATGTACAGTATTGCGTTTTGGGGAGAAGAAAGAGAGATGTAGGGCTAGAG
SEQ ID NO: 282:
AATGTACAGTATTGCGTTTTGGCAAGCACTTCTGTTTTTGTCTTTTCAGTTTCG
SEQ ID NO: 283:
AATGTACAGTATTGCGTTTTGTCTCTGATATACTTGGATTGGTAATTGAGAAAGTCT
SEQ ID NO: 284:
AATGTACAGTATTGCGTTTTGGTTTGATATCTTCCCAGCAAAATAATCAGCTCTCAT
SEQ ID NO: 285:
AATGTACAGTATTGCGTTTTGTAGCCAACCTCTTTTCGATGAGCTCACTAG
SEQ ID NO: 286:
AATGTACAGTATTGCGTTTTGTGGAACAGACAAACTATCGACTGAAGTTGT
SEQ ID NO: 287:
AATGTACAGTATTGCGTTTTGGAGGCTGAGTGCAAATTTGGTCTGGAA
SEQ ID NO: 288:
AATGTACAGTATTGCGTTTTGGATGGTGGTGGTTGTCTCTGATGATTACC
SEQ ID NO: 289:
AATGTACAGTATTGCGTTTTGGCAAGGCGAGTCCAGAACCAAGATT
SEQ ID NO: 290:
AATGTACAGTATTGCGTTTTGTCAGAAGCGACTGATCCCCATCAAGT
SEQ ID NO: 291:
AATGTACAGTATTGCGTTTTGCATATGGTCACATCACCTTAACTAAACCCATGTTT
SEQ ID NO: 292:
AATGTACAGTATTGCGTTTTGTTTCTCGGTACTGTTTATTTTGAACAAAACCAATCC
SEQ ID NO: 293:
AATGTACAGTATTGCGTTTTGCCTCCTCCCCAAATTCCAGGAACAATATGA
SEQ ID NO: 294:
AATGTACAGTATTGCGTTTTGTGTGCGTCATTTTATTTGGGAAAATTTGATACTAAC
SEQ ID NO: 295:
AATGTACAGTATTGCGTTTTGCATGCAGGAGAAGTCATCCCCCTTC
SEQ ID NO: 296:
AATGTACAGTATTGCGTTTTGTCTGAAAACTGGTGGTTGCCTCTAGGTTAA
SEQ ID NO: 297:
AATGTACAGTATTGCGTTTTGGCCCCTTTCTTGCTCTTCTTGGACTTG
SEQ ID NO: 298:
AATGTACAGTATTGCGTTTTGCCAAGCCAAGCCAAGCTGGATATTGTG
SEQ ID NO: 299:
AATGTACAGTATTGCGTTTTGCACTCACATTGTGCAGCTTGTAGTAGAG
SEQ ID NO: 300:
AATGTACAGTATTGCGTTTTGGCAAAGCGTCTGCATTTGAAGGAGTTT
SEQ ID NO: 301:
AATGTACAGTATTGCGTTTTGCCCTCCCGAGAACTTGCCGGTTAA
SEQ ID NO: 302:
AATGTACAGTATTGCGTTTTGGCTCCCCACCACAAAAACGCAAATG
SEQ ID NO: 303:
AATGTACAGTATTGCGTTTTGGTGTCACTGACGGAGAGCATGAAGATG
SEQ ID NO: 304:
AATGTACAGTATTGCGTTTTGCCACCCAAAGAAGTGTCTCCTGACC
SEQ ID NO: 305:
AATGTACAGTATTGCGTTTTGTCCGTCAGTGACACCTGGTACTTGAC
SEQ ID NO: 306:
AATGTACAGTATTGCGTTTTGCCCTAGCTCTGCCTACCCTGATCTTTC
SEQ ID NO: 307:
AATGTACAGTATTGCGTTTTGACGAGGTGGACGTCTTCTTCAATCAC
SEQ ID NO: 308:
AATGTACAGTATTGCGTTTTGGCCCTGCGAGTCGAGGTGATTG
SEQ ID NO: 309:
AATGTACAGTATTGCGTTTTGCCATGACTCTCAGGAATTGGCCCTATACTTAG
SEQ ID NO: 310:
AATGTACAGTATTGCGTTTTGCTTGGGACCTTCATTTCTATATAACCCCTATCTGG
SEQ ID NO: 311:
AATGTACAGTATTGCGTTTTGTGCCAGGAAACTTTTCATTGTGCCTCTC
SEQ ID NO: 312:
AATGTACAGTATTGCGTTTTGGTTACCCCATGGAACTTACCAAGCACTAG
SEQ ID NO: 313:
AATGTACAGTATTGCGTTTTGGTATGAAATTCGCTGGAGGGTCATTGAATCAAT
SEQ ID NO: 314:
AATGTACAGTATTGCGTTTTGCAGGAAGGAGCACTTACGTTTTAGCATCTTC
SEQ ID NO: 315:
AATGTACAGTATTGCGTTTTGGATTTTGAGAAATTCCCTTAATATCCCCATGCTCAA
SEQ ID NO: 316:
AATGTACAGTATTGCGTTTTGCACAACCACATGTGTCCAGTGAAAATCC
SEQ ID NO: 317:
AATGTACAGTATTGCGTTTTGTGCTTTCATCAGCAGGGTTCAATCCAAA
SEQ ID NO: 318:
AATGTACAGTATTGCGTTTTGCATTTACATCATCACAGAGTATTGCTTCTATGGAGA
SEQ ID NO: 319:
AATGTACAGTATTGCGTTTTGGTGATCTCTGGATGTCGGAATATTTAGAAACCTCT
SEQ ID NO: 320:
AATGTACAGTATTGCGTTTTGATCTTTTGAAAACAATGGTGACTACATGGACATGAA
SEQ ID NO: 321:
AATGTACAGTATTGCGTTTTGGGTCTAAAAAGGTCTGTGTTCCTTGAACTTACA
SEQ ID NO: 322:
AATGTACAGTATTGCGTTTTGCCAGCACCAATACATTTAATTTCTTTTCTGCAGAC
SEQ ID NO: 323:
AATGTACAGTATTGCGTTTTGGCTACAGATGGCTTGATCCTGAGTCATTTC
SEQ ID NO: 324:
AATGTACAGTATTGCGTTTTGGTCAGGCCCATACCAAGGGAAAAGATC
SEQ ID NO: 325:
AATGTACAGTATTGCGTTTTGACACTGAGTGATGTCTGGTCTTATGGCATT
SEQ ID NO: 326:
AATGTACAGTATTGCGTTTTGCACTGAGCGTTTGTTAGTCCTGGTGTTTT
SEQ ID NO: 327:
AATGTACAGTATTGCGTTTTGCAGATTCTCCACAATCTCACTCAGGTGGTAAA
SEQ ID NO: 328:
AATGTACAGTATTGCGTTTTGCCCCACAGCTACGAGATCATGGTGAAAT
SEQ ID NO: 329:
AATGTACAGTATTGCGTTTTGTCTCTATTCATTTTTGAGGTTTGGTTGTTAACACTT
SEQ ID NO: 330:
AATGTACAGTATTGCGTTTTGGGGAGTGCACCATTATCGGGAAAATGG
SEQ ID NO: 331:
AATGTACAGTATTGCGTTTTGGCTTATTCTCATTCGTTTCATCCAGGATCTCAAAA
SEQ ID NO: 332:
AATGTACAGTATTGCGTTTTGGGGCGACGAGATTAGGCTGTTATGC
SEQ ID NO: 333:
AATGTACAGTATTGCGTTTTGCCCCTCTGCATTATAAGCAGTGCCAAAA
SEQ ID NO: 334:
AATGTACAGTATTGCGTTTTGGCCCACATCGTTGTAAGCCTTACATTCAA
SEQ ID NO: 335:
AATGTACAGTATTGCGTTTTGCCGTTTGGAAAGCTAGTGGTTCAGAGTTC
SEQ ID NO: 336:
AATGTACAGTATTGCGTTTTGGAGATCCCATCCTGCCAAAGTTTGTGATT
SEQ ID NO: 337:
AATGTACAGTATTGCGTTTTGGGAAAGCCCCTGTTTCATACTGACCAAAA
SEQ ID NO: 338:
AATGTACAGTATTGCGTTTTGCTTTCTCCCCACAGAAACCCATGTATGAAG
SEQ ID NO: 339:
AATGTACAGTATTGCGTTTTGGTTTGCCAGTTGTGCTTTTTGCTAAAATGC
SEQ ID NO: 340:
AATGTACAGTATTGCGTTTTGCCCTCCCACCCTCAGGACTATACCAAT
SEQ ID NO: 341:
AATGTACAGTATTGCGTTTTGTGCTCGGCAGATTGGTATAGTCCTG
SEQ ID NO: 342:
AATGTACAGTATTGCGTTTTGGGCATCCTCTGTCCTATCTCCCAGATACA
SEQ ID NO: 343:
AATGTACAGTATTGCGTTTTGAGGTTTTATACTAAACTTACTTTGACTGGGTTTGG
SEQ ID NO: 344:
AATGTACAGTATTGCGTTTTGCCCCCAGAGGTAAGCGTCATATGG
SEQ ID NO: 345:
AATGTACAGTATTGCGTTTTGGCACAGGGAAGTAGGTACTGGGAGATTG
SEQ ID NO: 346:
AATGTACAGTATTGCGTTTTGAGGCCTGCAAGGTTTTAACTGGACCTA
SEQ ID NO: 347:
AATGTACAGTATTGCGTTTTGCGGGAGCTGATAAGTGGTACCTGTATGT
SEQ ID NO: 348:
AATGTACAGTATTGCGTTTTGGAAAAGGGTCCCAGGTAGGTCCAGTTAA
SEQ ID NO: 349:
AATGTACAGTATTGCGTTTTGCTCTCGGTGTATTTCTCTACTTACCTGTAATAATGC
SEQ ID NO: 350:
AATGTACAGTATTGCGTTTTGTTTATTGATGTCTATGAAGTGTTGTGGTTCCTTAAC
SEQ ID NO: 351:
AATGTACAGTATTGCGTTTTGCAGAAAACAAGCTGCCGCAAAGTTCTAC
SEQ ID NO: 352:
AATGTACAGTATTGCGTTTTGCAGGTGTTGCGATGATGTCACTGTACG
SEQ ID NO: 353:
AATGTACAGTATTGCGTTTTGTCATTTTTCATTGGACTTGTTTTGTCAGCTTTTTGG
SEQ ID NO: 354:
AATGTACAGTATTGCGTTTTGGTTAGCCCCAATATGAAAAATAAAGCTGGTTGGA
SEQ ID NO: 355:
AATGTACAGTATTGCGTTTTGCTGGTTGGAGGTTTTTGCTAAATCTGGAATGA
SEQ ID NO: 356:
AATGTACAGTATTGCGTTTTGTTCTTTTTGACTAGAAAACTTCAGCCACTGTGTATT
SEQ ID NO: 357:
AATGTACAGTATTGCGTTTTGCATATGACCAATTGCAGATGAGCCCATTATTGAA
SEQ ID NO: 358:
AATGTACAGTATTGCGTTTTGAGGCATAGCTGACTCATCTATGTTTGTTCT
SEQ ID NO: 359:
AATGTACAGTATTGCGTTTTGTTCCTCATTTCTTTCACTCTGACAGTATAAAGGTAA
SEQ ID NO: 360:
AATGTACAGTATTGCGTTTTGGAACTATTCCAACAGAACAAACCGATAACATCA
SEQ ID NO: 361:
AATGTACAGTATTGCGTTTTGTGGATAGCAAGACAATTAGAGCCCAACTTAGT
SEQ ID NO: 362:
AATGTACAGTATTGCGTTTTGCTACTCCTCCTGTCTCTTTCCACATCATCAATT
SEQ ID NO: 363:
AATGTACAGTATTGCGTTTTGAGGACCTTATGTTGTATGCTGTATAAATCTAAAGGT
SEQ ID NO: 364:
AATGTACAGTATTGCGTTTTGGTTTGTCATCTTCTATGGTAAGTATCTTTCTGGATG
SEQ ID NO: 365:
AATGTACAGTATTGCGTTTTGTGGAGGAGAAACAGATAAAAGTTGAGTATACGTTTA
SEQ ID NO: 366:
AATGTACAGTATTGCGTTTTGGAGGATGACGACATGTTAGTAAGCACTACTACT
SEQ ID NO: 367:
AATGTACAGTATTGCGTTTTGATTCCACCATCATTTCCTTCTCCAAAATTATCATCC
SEQ ID NO: 368:
AATGTACAGTATTGCGTTTTGCTCAAAAGCACTGCCTTCTCTCATTATCTCAC
SEQ ID NO: 369:
AATGTACAGTATTGCGTTTTGAATGTATTTGACCTTCTTTTAAAGTGACATCGATGT
SEQ ID NO: 370:
AATGTACAGTATTGCGTTTTGTGATGTTCCCAACTTCTTCTCTCATGGTTATCTC
SEQ ID NO: 371:
AATGTACAGTATTGCGTTTTGCCCTCTGATCCCTAGATAATTTATGGGTAGCTAGA
SEQ ID NO: 372:
AATGTACAGTATTGCGTTTTGCACGAAATGCAGGTTTTGGAATATGATTAATGTT
SEQ ID NO: 373:
AATGTACAGTATTGCGTTTTGGAACAATGTTCTACGCACATTTTGTTCTCAGTAAA
SEQ ID NO: 374:
AATGTACAGTATTGCGTTTTGTCCACGCTGCTCTCTAAATTACACTCGAA
SEQ ID NO: 375:
AATGTACAGTATTGCGTTTTGACGTAGAACACATTTCATTTTACTCCTCTTTGG
SEQ ID NO: 376:
AATGTACAGTATTGCGTTTTGGTCACATGAATGTAAATCAAGAAAACAGATGTTGTT
SEQ ID NO: 377:
AATGTACAGTATTGCGTTTTGTTCTGAACTATTTATGGACAACAGTCAAACAACAAT
SEQ ID NO: 378:
AATGTACAGTATTGCGTTTTGTGAAGCCATTGCGAGAACTTTATCCATAAGTATTTC
SEQ ID NO: 379:
AATGTACAGTATTGCGTTTTGGCCAGAGCACATGAATAAATGAGCATCCAT
SEQ ID NO: 380:
AATGTACAGTATTGCGTTTTGGGAAGCTCTCAGGGTACAAATTCTCAGATCAT
SEQ ID NO: 381:
AATGTACAGTATTGCGTTTTGCTCAGGGTACAAATTCTCAGATCATCAGTCCTC
SEQ ID NO: 382:
AATGTACAGTATTGCGTTTTGCTCTACACAAGCTTCCTTTCCGTCATGC
SEQ ID NO: 383:
AATGTACAGTATTGCGTTTTGCCCTTCAGATCTTCTCAGCATTCGAGAGATC
SEQ ID NO: 384:
AATGTACAGTATTGCGTTTTGAATCGAAGCGCTACCTGATTCCAATTCC
SEQ ID NO: 385:
AATGTACAGTATTGCGTTTTGCCGACCGTAACTATTCGGTGCGTTG
SEQ ID NO: 386:
AATGTACAGTATTGCGTTTTGACATTCTATCCAAGCTGTGTTCTATCTTGAGAAACT
SEQ ID NO: 387:
AATGTACAGTATTGCGTTTTGCGAGTGAGGGTTTTCGTGGTTCACATC
SEQ ID NO: 388:
AATGTACAGTATTGCGTTTTGCGTGGGTCCCAGTCTGCAGTTAAG
SEQ ID NO: 389:
AATGTACAGTATTGCGTTTTGGCTCAGAGCCGTTCCGAGATCTT
SEQ ID NO: 390:
AATGTACAGTATTGCGTTTTGGCGTTCCATCTCCCACTTGTCGTAGTT
SEQ ID NO: 391:
AATGTACAGTATTGCGTTTTGCTGGCCGAGTTGGTTCATCATCATTCAA
SEQ ID NO: 392:
AATGTACAGTATTGCGTTTTGTATGGTGTGTCCCCCAACTACGACAAG
SEQ ID NO: 393:
AATGTACAGTATTGCGTTTTGTGAAAAGCACTTCCTGAAATAATTTCACCTTCGTTT
SEQ ID NO: 394:
AATGTACAGTATTGCGTTTTGAGGTACTCCATGGCTGACGAGATCTG
SEQ ID NO: 395:
AATGTACAGTATTGCGTTTTGTTGCCTTTGTTCCAAGGTCCAATGTGT
SEQ ID NO: 396:
AATGTACAGTATTGCGTTTTGCGTCCCCGCATTCCAACGTCTC
SEQ ID NO: 397:
AATGTACAGTATTGCGTTTTGGGCGCGCCGTTTACTTGAAGG
SEQ ID NO: 398:
AATGTACAGTATTGCGTTTTGGCCTGGCGGTGCACACTATTCTG
SEQ ID NO: 399:
AATGTACAGTATTGCGTTTTGAGGTGCAGCCACAAAACTTACAGATGC
SEQ ID NO: 400:
AATGTACAGTATTGCGTTTTGGTGCCGAACCAATACAACCCTCTG
SEQ ID NO: 401:
AATGTACAGTATTGCGTTTTGGGGCGGGTCCACCAGTTTGAAT
SEQ ID NO: 402:
AATGTACAGTATTGCGTTTTGCCGCAGAGGGTTGTATTGGTTCG
SEQ ID NO: 403:
AATGTACAGTATTGCGTTTTGAGCCACTCGCATTGACCATTCAAACT
SEQ ID NO: 404:
AATGTACAGTATTGCGTTTTGCCACGTCTGACAGGTAGCCATGG
SEQ ID NO: 405:
AATGTACAGTATTGCGTTTTGGTGAGGCTGCTGGACGAGTACAAC
SEQ ID NO: 406:
AATGTACAGTATTGCGTTTTGCGCACCAGGTTGTACTCGTCCA
SEQ ID NO: 407:
AATGTACAGTATTGCGTTTTGCCGCCTTTGTGCTTCTGTTCTTCGT
SEQ ID NO: 408:
AATGTACAGTATTGCGTTTTGCTGATTAATCGCGTAGAAAATGACCTTATTTTGGAG
SEQ ID NO: 409:
AATGTACAGTATTGCGTTTTGGCTCCATCGTCTACCTGGAGATTGACAA
SEQ ID NO: 410:
AATGTACAGTATTGCGTTTTGTCTGCACGGCCTCGATCTTGTAGG
SEQ ID NO: 411:
AATGTACAGTATTGCGTTTTGGCCAGCAGATGATCTTCCCCTACTACG
SEQ ID NO: 412:
AATGTACAGTATTGCGTTTTGCGTCACGCTTGAAGACCACGTTG
SEQ ID NO: 413:
AATGTACAGTATTGCGTTTTGGCCAGCATGCAGTTCTAAGGCTCT
SEQ ID NO: 414:
AATGTACAGTATTGCGTTTTGGTGCCCGTCTCGACTCTTAGGC
SEQ ID NO: 415:
AATGTACAGTATTGCGTTTTGTGTAGCCGCTGATCGTCGTGTATATGTC
SEQ ID NO: 416:
AATGTACAGTATTGCGTTTTGGACTGGTACTGGTTAGTAAAGGTTGATAATATTCCA
SEQ ID NO: 417:
AATGTACAGTATTGCGTTTTGGGTGAAGTAATCAGTTTGTTCACTAGTTACGTGATT
SEQ ID NO: 418:
AATGTACAGTATTGCGTTTTGCTGACATGCCTACTGATTATTCTTCAAACTCATCAC
SEQ ID NO: 419:
AATGTACAGTATTGCGTTTTGTGTGTGTTTTAATTGTTCCACTTGAGATTCTTAACC
SEQ ID NO: 420:
AATGTACAGTATTGCGTTTTGCGTCAGCATTTTGAATCACTTCATTCTGACATGATA
SEQ ID NO: 421:
AATGTACAGTATTGCGTTTTGAGTAATTTTCAACTATTGGCCTAGTGAATTTAAGCT
SEQ ID NO: 422:
AATGTACAGTATTGCGTTTTGAGAAAGAGGGAAGTCACATTTATAGAGTGCTAGC
SEQ ID NO: 423:
AATGTACAGTATTGCGTTTTGCATCAACAGAAACAGAACAACAAACTGTGACAAATC
SEQ ID NO: 424:
AATGTACAGTATTGCGTTTTGCCAAAGAATATCCCTTTATATAGCAGTGGAACAATT
SEQ ID NO: 425:
AATGTACAGTATTGCGTTTTGCAGAATATGCAGTGATAAGTGCTGTTTCATCACT
SEQ ID NO: 426:
AATGTACAGTATTGCGTTTTGTTCCCCCTGTGACGACTACTTTTCCTC
SEQ ID NO: 427:
AATGTACAGTATTGCGTTTTGCGGTCCCTATTTCTTCCTCTGCTTCGT
SEQ ID NO: 428:
AATGTACAGTATTGCGTTTTGCTGAACAGTTCTGTCTCTATTACCCGACCTC
SEQ ID NO: 429:
AATGTACAGTATTGCGTTTTGCGTTCATAGCCTTCTATCCGAGTATGTAGCA
SEQ ID NO: 430:
AATGTACAGTATTGCGTTTTGCCCCTTCTGTCCTCGCAGGTTAATCC
SEQ ID NO: 431:
AATGTACAGTATTGCGTTTTGGCTTCCAGCCATTTCTGAGATATCCTCACAGT
SEQ ID NO: 432:
AATGTACAGTATTGCGTTTTGACCAGGAGGAACAAAGACACATGAAGATCAT
SEQ ID NO: 433:
AATGTACAGTATTGCGTTTTGGCGCCCCCGAGTTTCTTACGAATC
SEQ ID NO: 434:
AATGTACAGTATTGCGTTTTGTTTATACACAGTTTGGAGTTTGAGAATCAGAAGACT
SEQ ID NO: 435:
AATGTACAGTATTGCGTTTTGGGTTATCTCTGGCTGATGAGATTATGAGTGATTCTC
SEQ ID NO: 436:
AATGTACAGTATTGCGTTTTGGCCAAGCTAGTGATTGATGTGATTCGCTAT
SEQ ID NO: 437:
AATGTACAGTATTGCGTTTTGCCCCTCCTCTAGTACTCCCTGTTTGT
SEQ ID NO: 438:
AATGTACAGTATTGCGTTTTGCTCCTTCCTGTCCCAATCAACTAGTCTAGC
SEQ ID NO: 439:
AATGTACAGTATTGCGTTTTGGCCTCGTCCCTCTTCCCTTAGGTAA
SEQ ID NO: 440:
AATGTACAGTATTGCGTTTTGTCTCTCTTCCCATTAGTCTGAGTACTGAGTGATT
SEQ ID NO: 441:
AATGTACAGTATTGCGTTTTGAGCATTTCTTGAGACTTAAAGTGGCATTCTAAAGG
SEQ ID NO: 442:
AATGTACAGTATTGCGTTTTGATTTTTATTCTCAAGAGGCAGAAATACCAACTTACC
SEQ ID NO: 443:
AATGTACAGTATTGCGTTTTGAATTTATAGCTCTTTTCATCTGCTTTGGTATCATCA
SEQ ID NO: 444:
AATGTACAGTATTGCGTTTTGGCCTCTAATCTGATATACAGCCTTAGAAAGTCACA
SEQ ID NO: 445:
AATGTACAGTATTGCGTTTTGTGTGCCATTGTCCTGGAGCAACAATT
SEQ ID NO: 446:
AATGTACAGTATTGCGTTTTGAGTGTACTGCTCGTTTTCTTAATTTGAAAAGTGAGT
SEQ ID NO: 447:
AATGTACAGTATTGCGTTTTGACCCATGAACTAATACTTATTTTGAGATTGGTCCAT
SEQ ID NO: 448:
AATGTACAGTATTGCGTTTTGCATGGTGCAACAAAAGTAAGAATCCAACAGTTTT
SEQ ID NO: 449:
AATGTACAGTATTGCGTTTTGTTGAAATGTTAAGTAAGCTTGAAATACCGATAGCAT
SEQ ID NO: 450:
AATGTACAGTATTGCGTTTTGGGGAGGAAGAAAATGAAGCACGAGGAAAAC
SEQ ID NO: 451:
AATGTACAGTATTGCGTTTTGATTTGGGATGTACTCTAAATTTAAAGCAGCAAATCA
SEQ ID NO: 452:
AATGTACAGTATTGCGTTTTGTCAAGAGCAGAATTTGGAGACTTTGATATTAAAACT
SEQ ID NO: 453:
AATGTACAGTATTGCGTTTTGCGGTTACTAACATGTTTAGGGAAATAGACAACTGTT
SEQ ID NO: 454:
AATGTACAGTATTGCGTTTTGCCTGACAACAGATCCCATATAATTAACTTTCATACC
SEQ ID NO: 455:
AATGTACAGTATTGCGTTTTGAGATGAAGAAGATGAGGAACGAGAGAGTAAAAGC

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference. The entirety of U.S. Appl. No. 62/790,338, filed Jan. 9, 2019, is incorporated herein by reference.

1. A method for detecting an analyte in a sample, comprising:
attaching first and second proximity probes to an analyte in the sample, wherein the first proximity probe comprises a first analyte binding domain and a first oligonucleotide domain comprising a universal amplification region, a variable probe specific tag region (PST), a unique molecular identifier (UMI), and an inter-molecular reacting region (IMR), and wherein the second proximity probe comprises a second analyte binding domain and a second oligonucleotide domain comprises a universal amplification region, a PST, and an IMR; and
detecting the analyte.

2. The method of claim 1, wherein the oligonucleotide domain of the second proximity probe further comprises a UMI.

3. The method of claim 1, wherein the first and second analyte binding domains are antibodies, aptamers, ligands, receptors, or a combination thereof.

4. The method of claim 1, wherein the first and second analyte binding domains are conjugate to the oligonucleotide domains by a chemical bond, hybridization to an intermediary oligonucleotide linked to the analyte binding domain, streptavidin, biotin, or a combination thereof.

5. The method of claim 1, wherein the first and second analyte binding domains are first and second antibodies, respectively.

6. (canceled)

7. The method of claim 1, further comprising performing a proximity ligation (PLA) or extension (PEA) assay.

8. The method of claim 7, wherein the PLA or PEA assay generates a third oligonucleotide that is single-stranded or double-stranded.

9. The method of claim 8, further comprising attaching an adapter sequence to the third oligonucleotide.

10. The method of claim 9, wherein the adapter sequence is attached to the third oligonucleotide by amplification or ligation.

11. The method of claim 8, further comprising performing amplification of the third oligonucleotide to generate a protein-based DNA library.

12. The method of claim 1, further comprising preparing DNA and cDNA libraries from the sample, comprising:
ligating a DNA tag to an end of a DNA molecule in the sample, wherein the DNA tag comprises a UMI and a DNA identifier; and
performing reverse transcription of a RNA molecule in the sample in the presence of a RNA tag, wherein the RNA tag comprises a RNA identifier, a UMI, and a poly(T).

13. The method of claim 12, wherein the reverse transcription is performed in the presence of a second RNA tag, wherein the second RNA tag comprises a RNA identifier, a UMI, and a template switching oligonucleotide (TSO).

14. The method of claim 12, further comprising amplifying the tagged DNA and the tagged cDNA for enrichment with a set of gene specific primers.

15.-22. (canceled)

23. The method of claim 12, further comprising amplifying the second sample with primers specific for the RNA tag.

24.-30. (canceled)

31. The method of claim 1, further comprising:
(a) obtaining purified DNA and RNA from the same biological sample;
(b) attaching a DNA tag sequence to the DNA in the sample;
(c) attaching an RNA tag sequence to the RNA in the sample; and
(d) detecting DNA, RNA and protein targets, respectively.

32. A protein-based DNA library made by the method of claim 1.

33. A DNA library made by the method of claim 12.

34. A cDNA library made by the method of claim 12.

35. A composition comprising a first proximity probe comprising a first analyte binding domain and a first oligonucleotide domain comprising a universal amplification region, a variable probe specific tag region (PST), a unique molecular identifier (UMI), and an inter-molecular reacting region (IMR), and a second proximity probe comprising a second analyte binding domain and a second oligonucleotide domain comprises a universal amplification region, a PST, and an IMR.

36. The composition of claim 35, wherein the second oligonucleotide domain further comprises a unique molecular identifier (UMI).

37.-45. (canceled)
